(19) United States
(12) Patent Application Publication
(10) Pub. No.: US 2025/0257051 A1
(43) Pub. Date: Aug. 14, 2025

(54) SUBSTITUTED PYRIMIDINE COMPOUNDS AS TYK2 INHIBITORS

(71) Applicant: 1910 Genetics Inc., Boston, MA (US)

(72) Inventors: Jennifer NWANKWO, Boston, MA (US); Lewis WHITEHEAD, Swampscott, MA (US); Robert YOUNG, Bedford (GB)

(21) Appl. No.: 19/039,366

(22) Filed: Jan. 28, 2025

Related U.S. Application Data
(63) Continuation of application No. PCT/US2023/071157, filed on Jul. 27, 2023.
(60) Provisional application No. 63/369,760, filed on Jul. 28, 2022.

(51) Int. Cl. C07D 401/14; A61K 31/506; A61K 31/5377; A61K 31/551; C07D 401/04; C07D 403/04; C07D 403/14

(52) U.S. Cl. CPC C07D 401/14; A61K 31/506; A61K 31/5377; A61K 31/551; C07D 401/04; C07D 403/04; C07D 403/14

(57) ABSTRACT

Provided are TYK2 inhibitors of Formula I, processes for their production, their use as pharmaceuticals and pharmaceutical compositions comprising them. These compounds are useful, for example, in treating TYK2-mediated disorders, such as, autoimmune and inflammatory disorders, metabolic disorders, proliferative disorders, endocrine disorders, neurological disorders, allergic disorders, and disorders associated with transplantation.

This application claims priority to U.S. Provisional Application No. 63/369,760 filed Jul. 28, 2022, which is hereby incorporated by reference in its entirety.

### TECHNICAL FIELD

Provided are novel compounds of Formula I as described below, processes for their production, their use as pharmaceuticals and pharmaceutical compositions comprising them. These compounds are useful in treating TYK2-mediated disorders, such as, autoimmune and inflammatory disorders, metabolic disorders, proliferative disorders, endocrine disorders, neurological disorders, allergic disorders, and disorders associated with transplantation.

### BACKGROUND

The search for new therapeutic agents has been greatly aided in recent years by a better understanding of the structure of enzymes and other biomolecules associated with diseases. The protein kinase family is an important class of enzymes that has been of interest and has been studied extensively.

Protein kinases constitute a large family of structurally related enzymes that are responsible for the control of a variety of signal transduction processes within the cell. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. An extracellular stimulus may affect one or more cellular responses related to cell growth, migration, differentiation, secretion of hormones, activation of transcription factors, muscle contraction, glucose metabolism, control of protein synthesis, and regulation of the cell cycle.

TYK2 is a non-receptor tyrosine kinase member of the Janus kinase (JAKs) family of protein kinases. The mammalian JAK family consists of four members, TYK2, JAK 1, IAK2, and JAK3. JAK proteins, including TYK2, are integral to cytokine signaling. TYK2 associates with the cytoplasmic domain of type I and type 11 cytokine receptors, as well as interferon types I and III receptors, and is activated by those receptors upon cytokine binding. Cytokines implicated in TYK2 activation include interferons (e.g., IFN-α, IFN-β, IFN-κ, IFN-δ, IFN-ε, IFN-τ, IFN-ω, and IFN-ζ (also known as limitin)) and interleukins (e.g. IL-4, IL-6, HL-10, IL-11, IL-12, IL-1.3, L-22, IL-23, IL-27, IL-31, oncostatin M, ciliary neurotrophic factor, cardiotrophin 1, cardiotrophin-like cytokine, and LIF). The activated TYK2 then goes on to phosphorylate further signaling proteins such as members of the STAT family, including STAT1, STAT2, STAT4, and STAT6.

Compounds that inhibit the activity of TYK2 are beneficial, especially those with selectivity over JAK2. Such compounds should deliver a pharmacological response that favorably treats one or more of the conditions described herein without the side-effects associated with the inhibition of JAK2. Accordingly there is a need to provide novel inhibitors having more effective or advantageous pharmaceutically relevant properties, like selectivity over other JAK kinases (especially JAK2).

Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### BRIEF SUMMARY

Provided are novel 2,4-diamine-5-carboxamide pyrimidines, in free or pharmaceutically acceptable salt form, processes for their production, their use as pharmaceuticals, for instance as TYK2 inhibitors, and pharmaceutical compositions comprising them.

Further provided is a compound of Formula I:

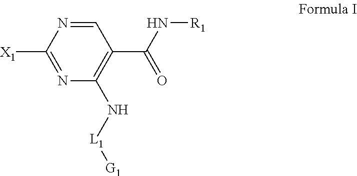

wherein:
R₁ is H, OH, or C₁₋₃-alkyl;
L₁ is a bond or carbonyl;
G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens;
R₂ is a 5- to 6-membered saturated heterocyclyl;
X₁ is

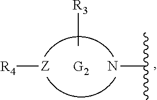

wherein the wavy line is the point of attachment of the fragment to Formula I;
G₂ is a 4- to 12-membered saturated heterocyclyl;
Z is CH or N;
R₃ is H or C₁₋₃-alkyl;
R₄ is H or —C(O)R₅; and
R₅ is C₁₋₃-alkyl, OH, NH₂, —NHC₁₋₃-alkyl, C₁₋₆-alkoxy, or C₃₋₆-cycloalkyl;
in free or pharmaceutically acceptable salt form.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the embodiment(s) is merely exemplary in nature and is in no way intended to limit the invention, its application, or uses.

All references cited herein are hereby incorporated by referenced in their entireties. In the event of a conflict in a definition in the present disclosure and that of a cited reference, the present disclosure controls.

In a first embodiment, provided is a compound of Formula I:

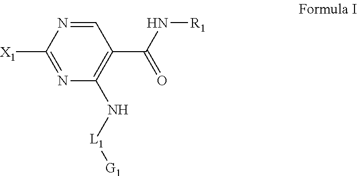

wherein:
R₁ is H, OH, or C₁₋₃-alkyl;
L₁ is a bond or carbonyl;
G₁ is an aryl or a 6- to 9-membered heteroaryl each optionally substituted with one or more substituents selected from —SC₁₋₃-alkyl, —SO₂Cl₃-alkyl, C₁₋₃-alkoxy, C₃₋₆-cycloalkyl, —C(O)R₂, halogen, and 5- to 6-membered heteroaryl optionally substituted with one or more halogens;
R₂ is a 5- to 6-membered saturated heterocyclyl;
X₁ is

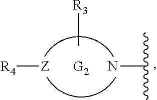

wherein the wavy line is the point of attachment of the fragment to Formula I;
G₂ is a 4- to 12-membered saturated heterocyclyl;
Z is CH or N;
R₃ is H or C₁₋₃-alkyl;
R₄ is H or —C(O)R₅; and
R₅ is C₁₋₃-alkyl, OH, NH₂, —NHC₁₋₃-alkyl, C₁₋₆-alkoxy, or C₃₋₆-cycloalkyl;
in free or pharmaceutically acceptable salt form.

In further embodiments, provided is a compound of Formula I, in free or pharmaceutically acceptable salt form, as follows:
1.1 Formula I, wherein R₁ is H.
1.2 Formula I, wherein R₁ is —CH₃.
1.3 Any of Formula I, 1.1, or 1.2, wherein L₁ is a bond.
1.4 Any of Formula I, 1.1, or 1.2, wherein L₁ is carbonyl.
1.5 Any of Formula I or 1.1-1.4, wherein G₁ is pyridinyl, phenyl, or indazolyl.
1.6 Any of Formula I or 1.1-1.5, wherein G₁ is pyridinyl. For instance, any of Formula I or 1.1-1.5, wherein G₁ is a monosubstituted pyridinyl.
1.7 Formula 1.6, wherein the pyridinyl is mono-substituted with cyclopropyl.
1.8 Formula 1.6 or 1.7, wherein the pyridinyl is:

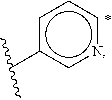

wherein the wavy line shows the point of attachment of the fragment to Formula I and the asterisk shows the carbon with substitution.
1.9 Any of Formula I or 1.1-1.5, wherein G₁ is phenyl.
1.10 Formula 1.9, wherein the phenyl is substituted with one or more substituents selected from —SO₂CH₃, C₁₋₃-alkoxy, —C(O)R₂, halogen, and 6-membered heteroaryl substituted with one or more halogens.
1.11 Formula 1.9 or 1.10, wherein the phenyl is substituted with —SO₂CH₃.
1.12 Formula 1.9 or 1.10, wherein the phenyl is substituted with methoxy.
1.13 Formula 1.11 or 1.12, wherein the phenyl is:

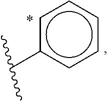

wherein the wavy line shows the point of attachment of the fragment to Formula I and the asterisk shows the carbon with substitution.
1.14 Formula 1.9, 1.10, or 1.12, wherein the phenyl is substituted with methoxy and

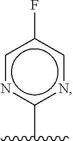

wherein the wavy line shows the point of attachment of the fragment to the phenyl.
1.15 Formula 1.9 or 1.10, wherein the phenyl is mono-substituted with —C(O)-morpholin-4-yl.
1.16 Any of Formula I or 1.1-1.5, wherein G₁ is indazolyl.
1.17 Formula 1.16, wherein the indazolyl is mono-substituted with methoxy.
1.18 Any of Formula I or 1.1-1.5, wherein G₁ is:

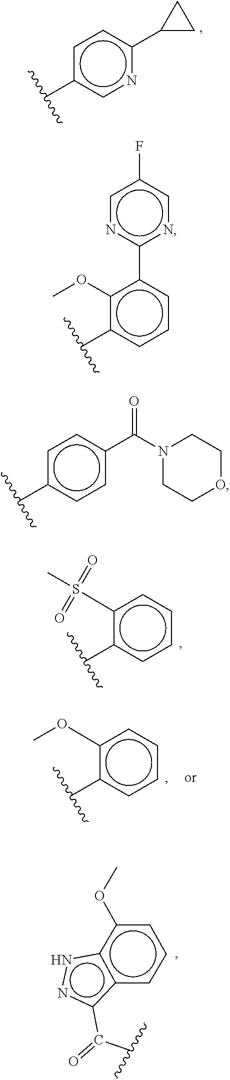

wherein the wavy line shows the point of attachment of the fragment to Formula I.
1.19 Any of Formula I or 1.1-1.18, wherein X₁ is:

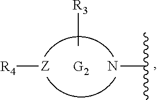

wherein the wavy line is the point of attachment of the fragment to Formula I.
1.20 Formula 1.19, wherein G₂ is pyrrolidinyl, piperazinyl, piperdinyl, diazepanyl, or azetidinyl.
1.21 Any of Formula I or 1.1-1.20, wherein R₃ is H.
1.22 Any of Formula I or 1.1-1.20, wherein R₃ is —CH₃.
1.23 Any of Formula I or 1.1-1.22, wherein R₄ is H.
1.24 Any of Formula I or 1.1-1.22, wherein R₄ is —C(O)R₅.
1.25 Formula 1.24, wherein R₅ is —NHCH₃, —CH₃, —OC(CH₃)₃, —OCH₂CH₃, or —OH.
1.26 Formula 1.24 or 1.25, wherein R₅ is —NHCH₃, —OC(CH₃)₃, —OCH₂CH₃, or —OH. For instance, Formula 1.24 or 1.25, wherein R₅ is —NHCH₃.
1.27 Any of Formula I or 1.1-1.26, wherein

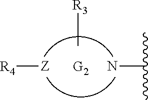

is:

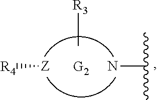

wherein the wavy line shows the point of attachment of the fragment to Formula I.
1.28 Any of Formula I or 1.1-1.26, wherein

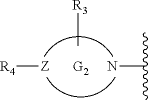

is:

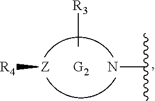

wherein the wavy line shows the point of attachment of the fragment to Formula I.
1.29 Any of Formula I or 1.1-1.28, wherein

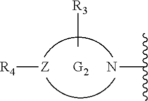

is:

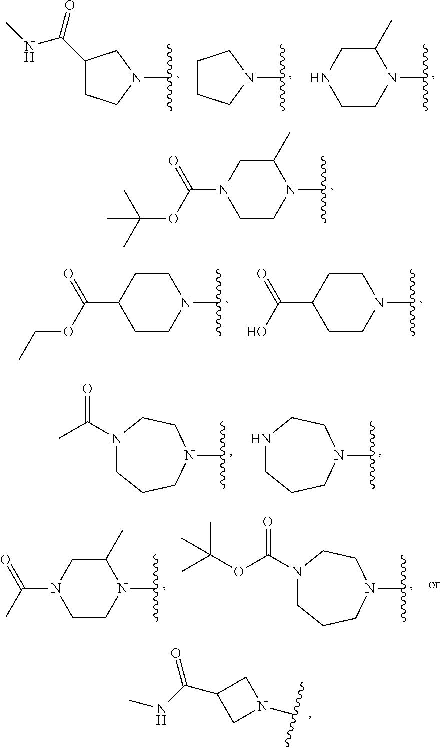

wherein the wavy line shows the point of attachment of the fragment to Formula 1.
1.30 Any of Formula I or 1.1-1.29, wherein

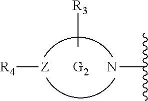

is:

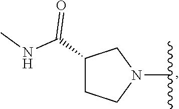 [SMILES: *N1CC[C@H](C(=O)NC)C1]

wherein the wavy line shows the point of attachment of the fragment to Formula I.
1.31 Any of Formula I or 1.1-1.29, wherein

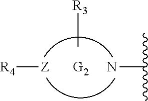

is:

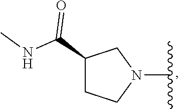 [SMILES: *N1CC[C@@H](C(=O)NC)C1]

wherein the wavy line shows the point of attachment of the fragment to Formula 1.
1.32 Any of Formula I or 1.1-1.31, wherein the compound, in free or pharmaceutically acceptable salt form, is selected from those set forth in Table 1 below:

TABLE 1

| Cmpd | |
|---|---|
| No. | Structure |
| 1 | 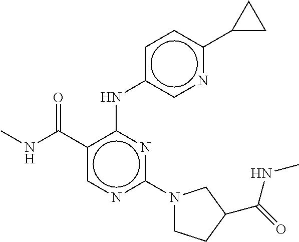 |
| 2 | 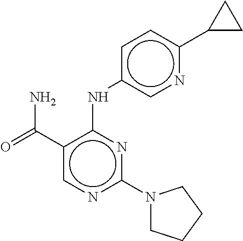 |
| 3 | 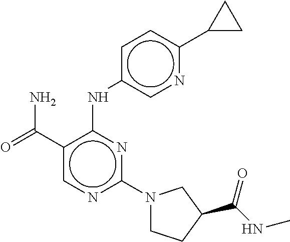 |
| 4 | 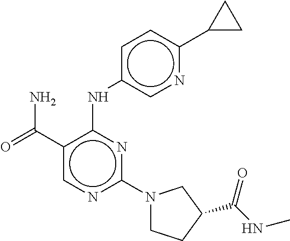 |
| 5 | 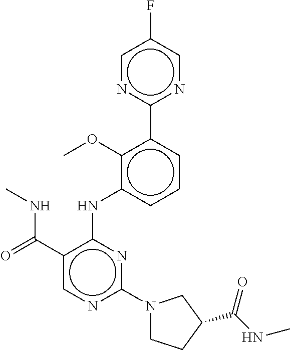 |
| 6 | 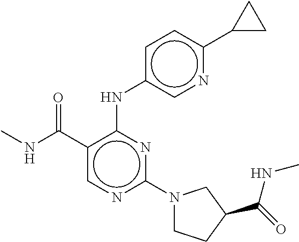 |
| 7 | 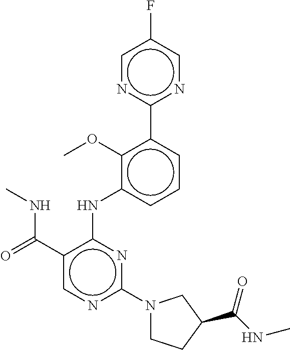 |
| 8 | 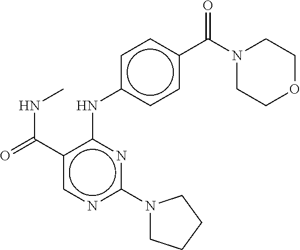 |
| 9 | 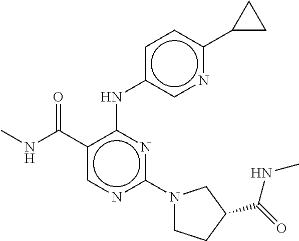 |
| 10 | 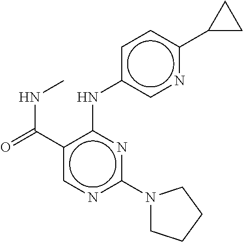 |
| 11 | 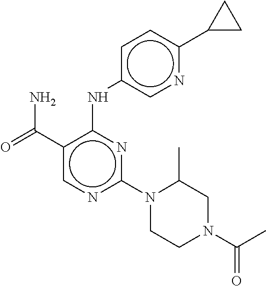 |
| 12 | 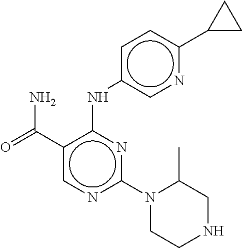 |
| 13 | 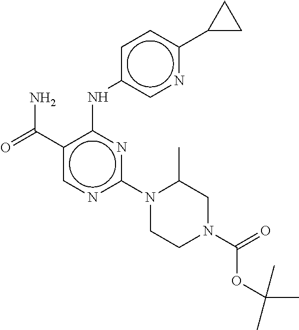 |
| 14 | 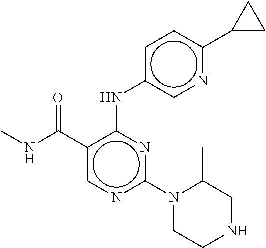 |
| 15 | 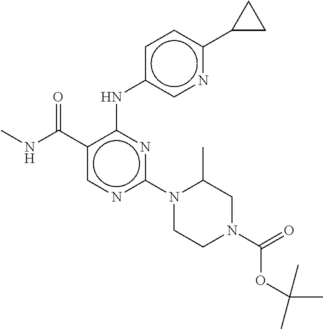 |
| 16 | 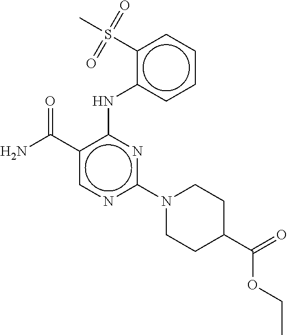 |
| 17 | 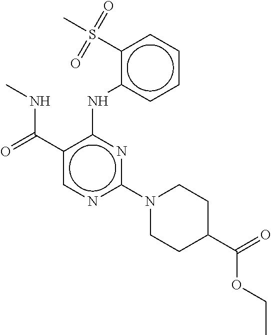 |
| 18 | 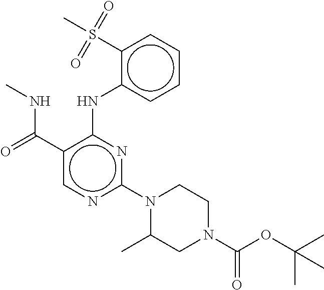 |
| 19 | 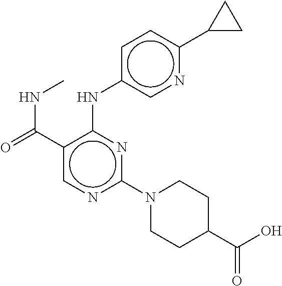 |
| 20 | 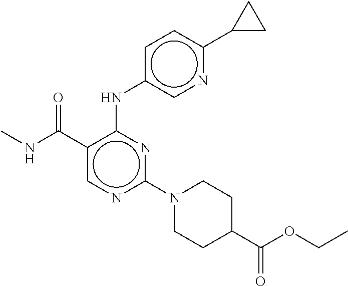 |
| 21 | 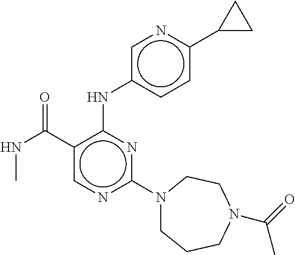 |
| 22 | 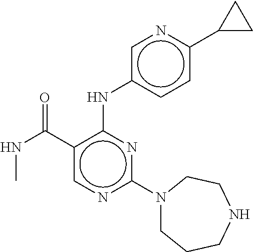 |
| 23 | 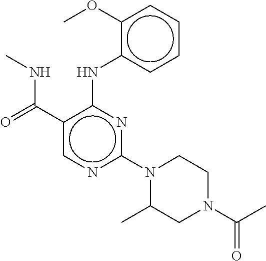 |
| 24 | 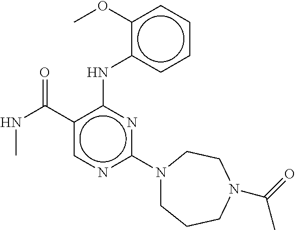 |
| 25 | 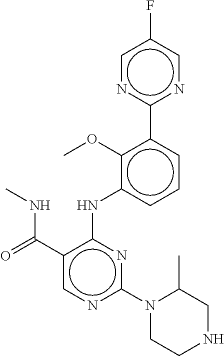 |
| 26 | 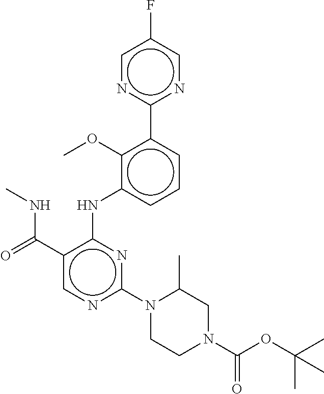 |
| 27 | 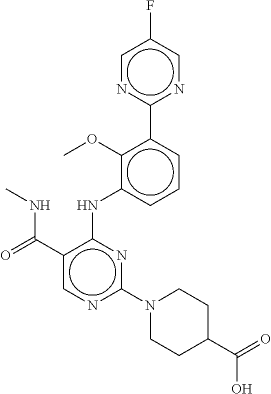 |
| 28 | 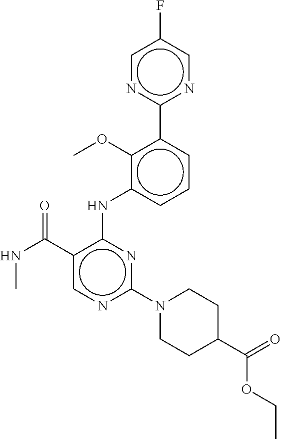 |
| 29 | 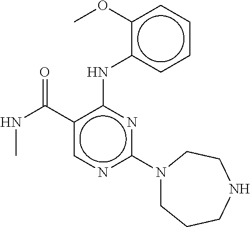 |
| 30 | 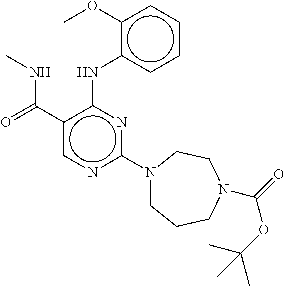 |
| 31 | 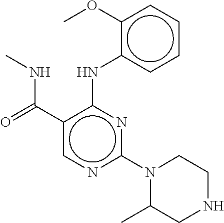 |
| 32 | 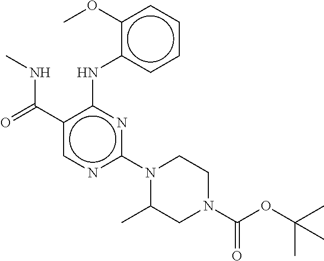 |
| 33 | 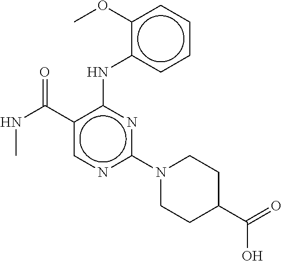 |
| 34 | 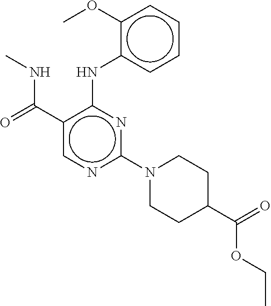 |
| 35 | 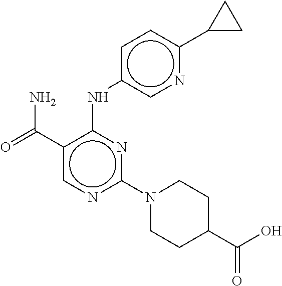 |
| 36 | 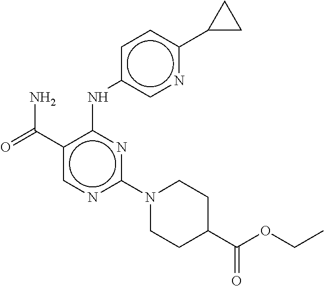 |
| 37 | 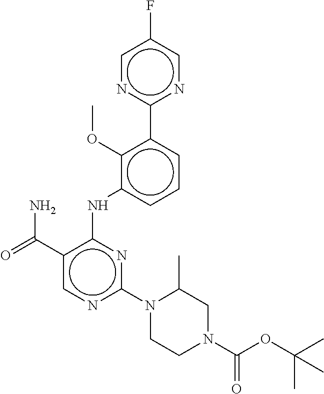 |
| 38 | 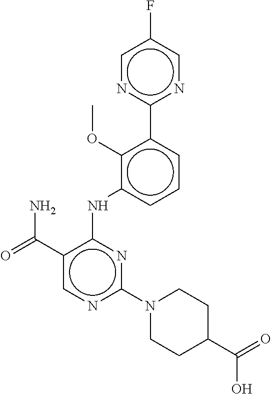 |
| 39 | 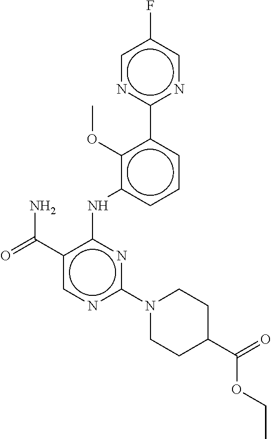 |
| 40 | 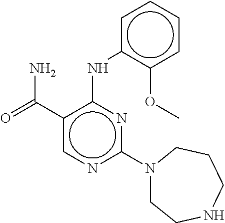 |
| 41 | 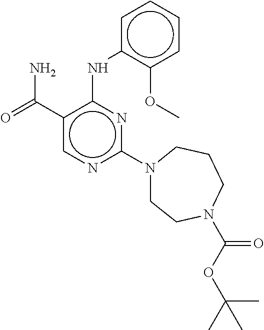 |
| 42 | 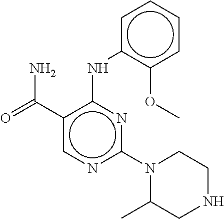 |
| 43 | 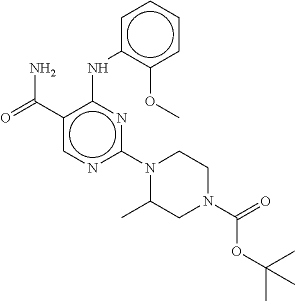 |
| 44 | 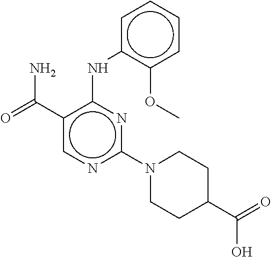 |
| 45 | 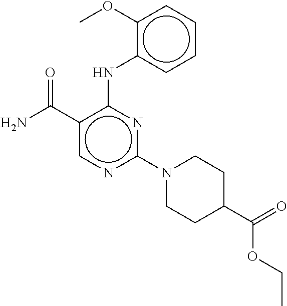 |
| 46 | 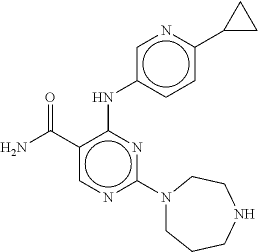 |
| 47 | 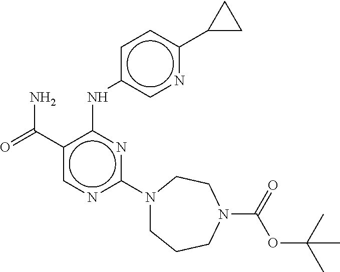 |
| 48 | 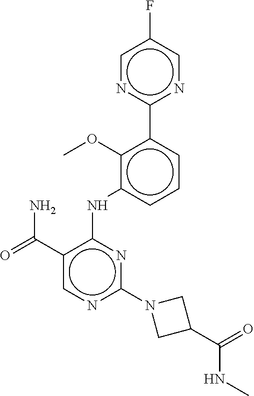 |
| 49 | 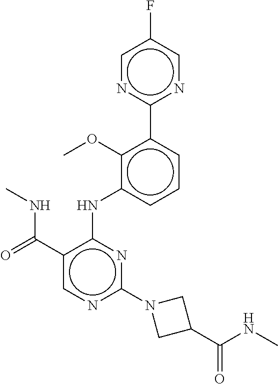 |
| 50 | 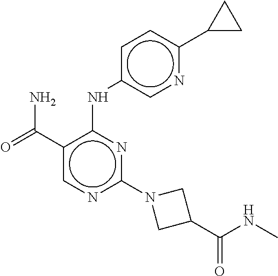 |
| 51 | 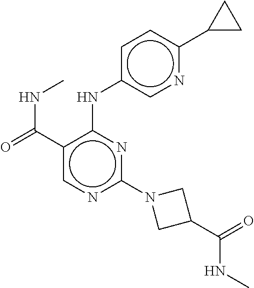 |
| 52 | 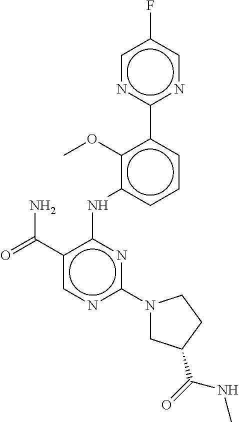 |
| 53 | 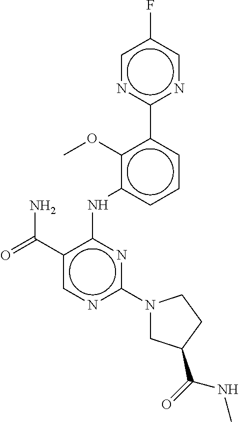 |
| 54 | 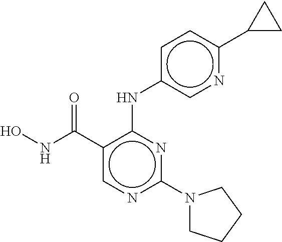 |
| 55 | 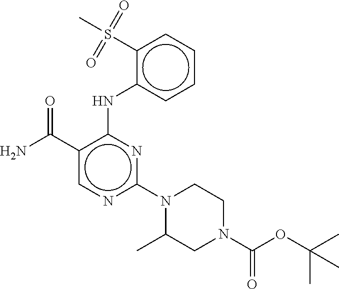 |
| 56 | 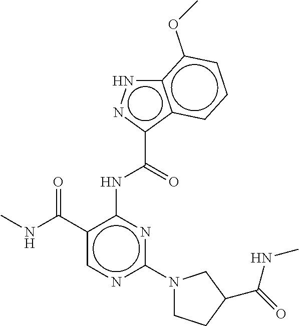 |
| 57 | 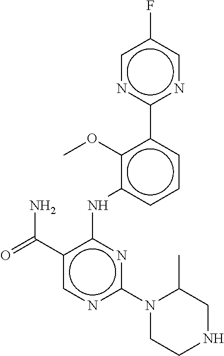 |
| 58 | 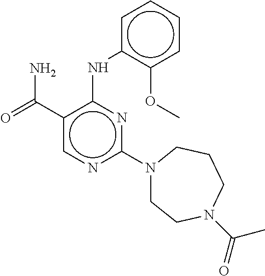 |
| 59 | 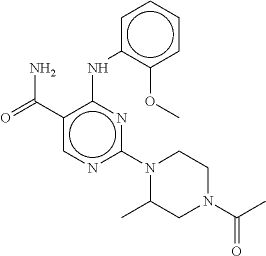 |
| 60 | 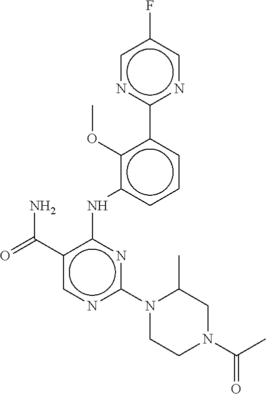 |
| 61 | 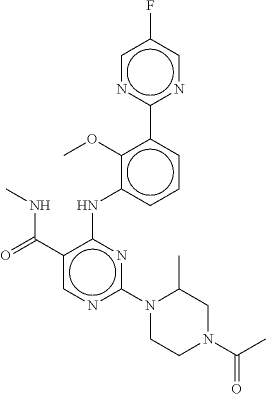 |
| 62 | 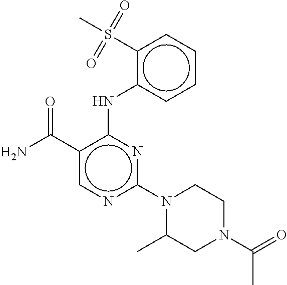 |
| 63 | 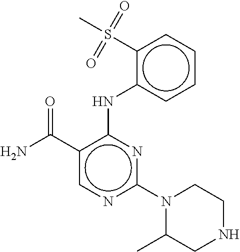 |
| 64 | 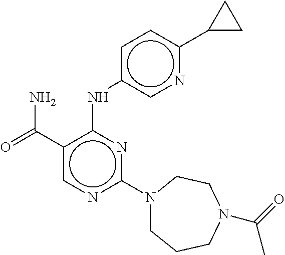 |
| 65 | 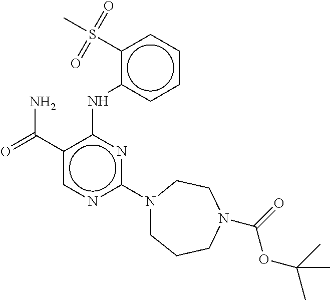 |
| 66 | 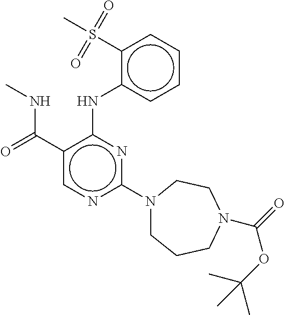 |
| 67 | 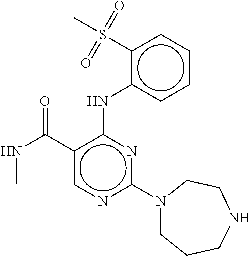 |
| 68 | 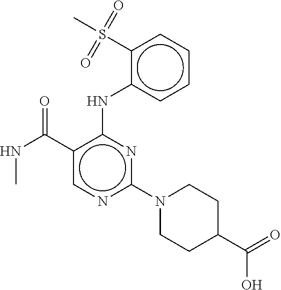 |
| 69 | 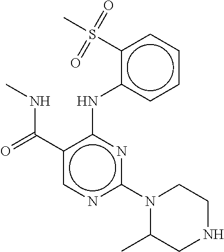 |
| 70 | 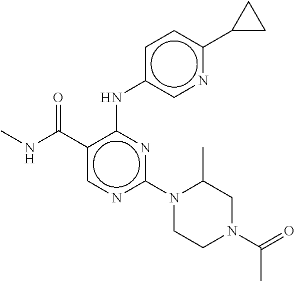 |
| 71 | 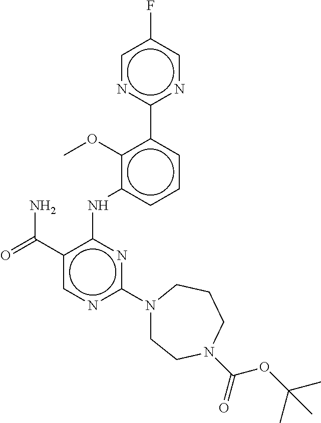 |
| 72 | 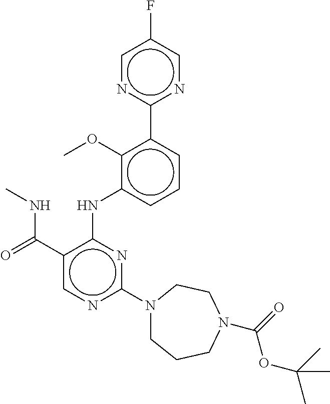 |
| 73 | 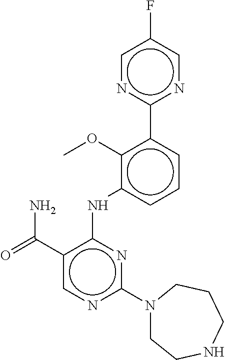 |
| 74 | 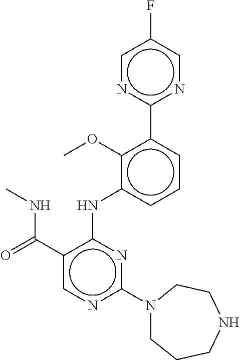 |
| 75 | 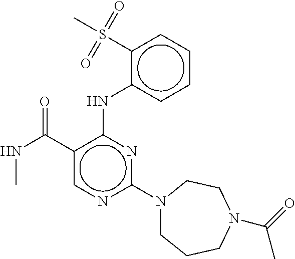 |
| 76 | 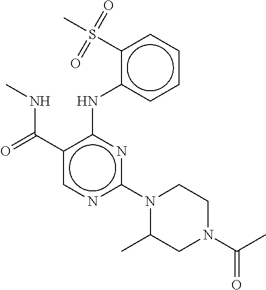 |
| 77 | 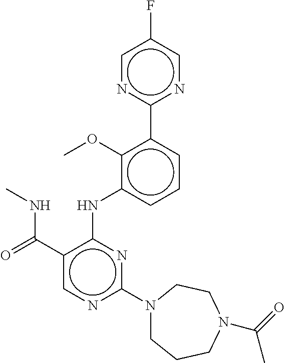 |
| 78 | 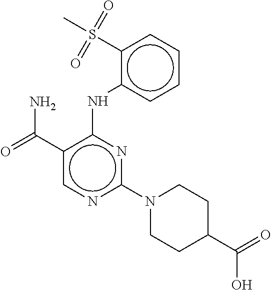 |
| 79 | 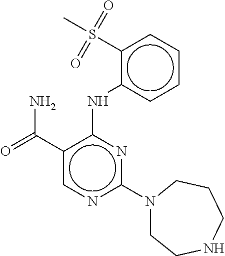 |
| 80 | 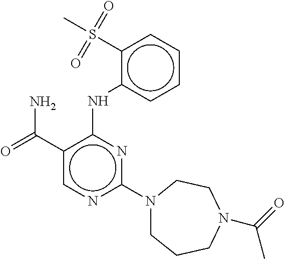 |
| 81 | 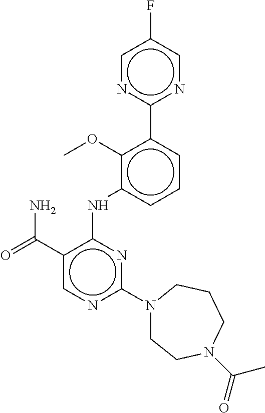 |

1.33 Any of Formula I or 1.1-1.32, wherein the compound, in free or pharmaceutically acceptable salt form, is selected from those set forth in Table 2 below:

TABLE 2

| Cmpd. | |
|---|---|
| No. | Structure |
| 2 | 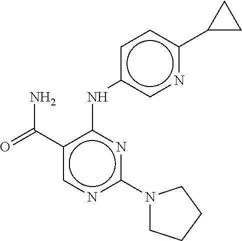 |
| 4 | 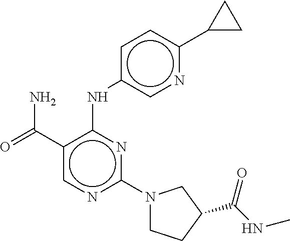 |
| 5 | 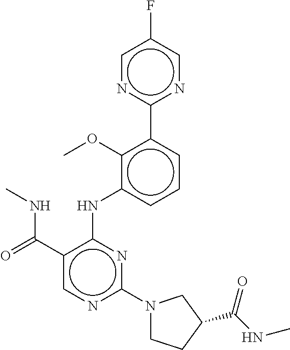 |
| 7 | 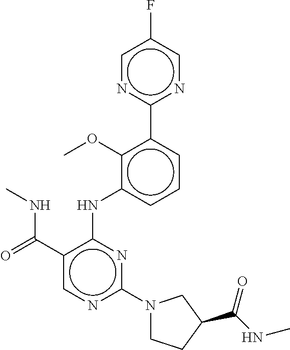 |
| 16 | 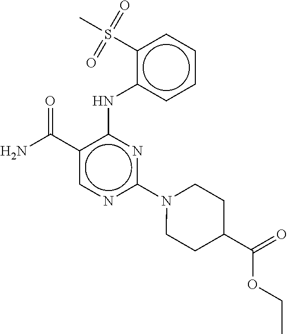 |
| 17 | 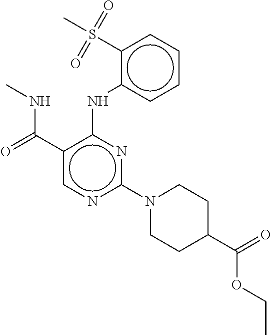 |
| 27 | 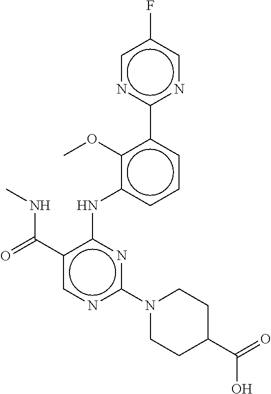 |
| 28 | 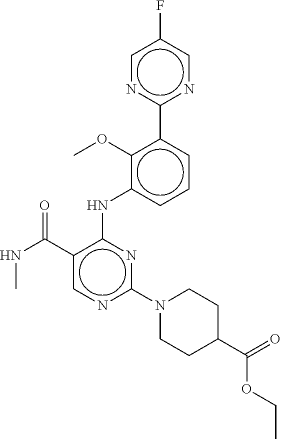 |
| 37 | 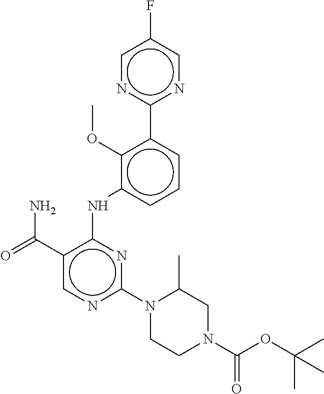 |
| 38 | 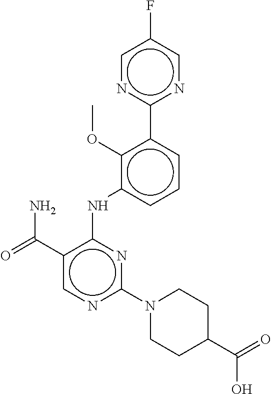 |
| 39 | 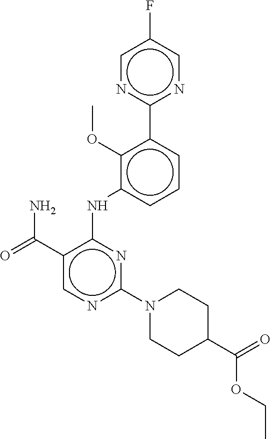 |
| 48 | 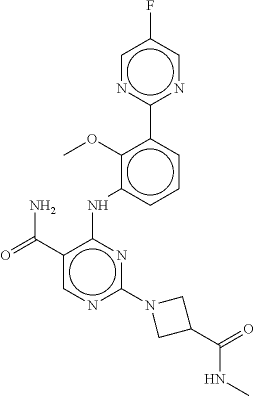 |
| 49 | 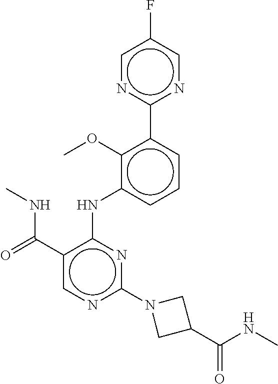 |
| 52 | 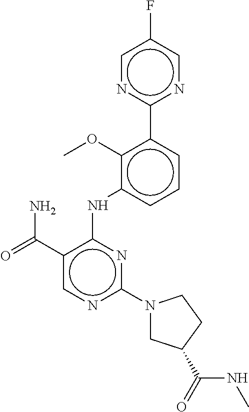 |
| 53 | 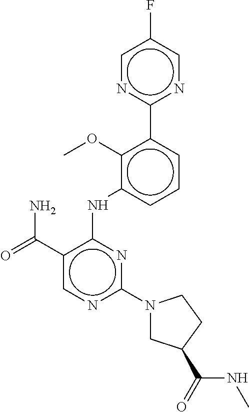 |
| 57 | 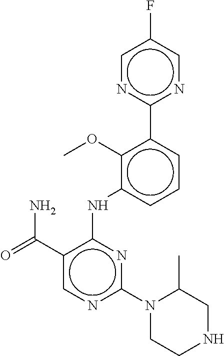 |
| 60 | 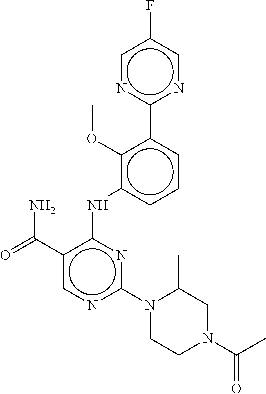 |
| 61 | 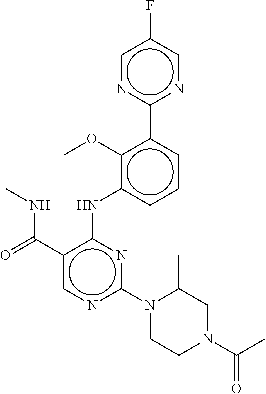 |

in free or pharmaceutically acceptable salt form.
1.34 Any of Formula I or 1.1-1.33, wherein the compound has a TYK2 Kd of less than 1 μM, e.g., less than 500 nm, e.g., less than 50 nM, e.g., less than 10 nM, e.g., equal to or less than 5 nM, measured, for instance, as described in Example 50.
1.35 Any of Formula I or 1.1-1.34, wherein the compound is in free form.
1.36 Any of Formula I or 1.1-1.34, wherein the compound is in pharmaceutically acceptable salt form.
1.37 Any of Formula I or 1.1-1.36, wherein the compound is in a pharmaceutical composition in combination with a pharmaceutically acceptable carrier. For instance, any of Formula I or 1.1-1.36, wherein a therapeutically effective amount of the compound is in a pharmaceutical composition in combination with a pharmaceutically acceptable carrier.

If not otherwise specified or clear from context, the following terms herein have the following meanings:

### Definitions and Conventions Used

Terms that are not specifically defined here have the meanings that would be apparent to a person skilled in the art, in the light of the overall disclosure and the context as a whole.

As used herein, the following definitions apply, unless stated otherwise:

The use of the prefix C_{x-y}, wherein x and y each represent a natural number, indicates that the chain or ring structure or combination of chain and ring structure as a whole, specified and mentioned in direct association, may consist of a maximum of y and a minimum of x number of carbon atoms.

In general, for groups comprising two or more subgroups, unless otherwise indicated the last named subgroup is the radical attachment point, for example, the substituent “aryl-C₁₋₃-alkyl” means an aryl group which is bound to a C₁-3-alkyl-group, the latter of which is bound to the core or to the group to which the substituent is attached. However, if a bond is depicted just prior to the first named subgroup, then that first named subgroup is the radical attachment point, for example, the substituent “—S(O)ₙC₁₋₆-alkyl” means a C₁₋₆-alkyl-group which is bound to an S(O)ₙ group, the latter of which is bound to the core or to the group to which the substituent is attached.

As used herein, alkyl denotes monovalent, saturated hydrocarbon chains, which may be present in both straight-chain (unbranched) and branched form. If an alkyl is substituted, the substitution may take place independently of one another, by mono- or polysubstitution in each case, on all the hydrogen-carrying carbon atoms. The term “C₁₋₅alkyl” includes, for example, H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—. Further examples of alkyl are methyl (Me; —CH₃), ethyl (Et; —CH₂CH₃), 1-propyl (n-propyl; n-Pr; —CH₂CH₂CH₃), 2-propyl (i-Pr; iso-propyl; —CH(CH₃)₂), 1-butyl (n-butyl; n-Bu; —CH₂CH₂CH₂CH₃), 2-methyl-1-propyl (iso-butyl; i-Bu; —CH₂CH(CH₃)₂), 2-butyl (sec-butyl; sec-Bu; —CH(CH₃)CH₂CH₃), 2-methyl-2-propyl (tert-butyl; t-Bu; —C(CH₃)₃), 1-pentyl (n-pentyl; —CH₂CH₂CH₂CH₂CH₃), 2-pentyl (—CH(CH₃)CH₂CH₂CH₃), 3-pentyl (—CH(CH₂CH₃)₂), 3-methyl-1-butyl (iso-pentyl; —CH₂CH₂CH(CH₃)₂), 2-methyl-2-butyl (—C(CH₃)₂CH₂CH₃), 3-methyl-2-butyl (—CH(CH₃)CH(CH₃)₂), 2,2-dimethyl-1-propyl (neo-pentyl; —CH₂C(CH₃)₃), 2-methyl-1-butyl (—CH₂CH(CH₃)CH₂CH₃), 1-hexyl (n-hexyl; —CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), 2,3-dimethyl-1-butyl (—CH₂CH(CH₃)CH(CH₃)CH₃), 2,2-dimethyl-1-butyl (—CH₂C(CH₃)₂CH₂CH₃), 3,3-dimethyl-1-butyl (—CH₂CH₂C(CH₃)₃), 2-methyl-1-pentyl (—CH₂CH(CH₃)CH₂CH₂CH₃), 3-methyl-1-pentyl (—CH₂CH₂CH(CH₃)CH₂CH₃), 1-heptyl (n-heptyl), 2-methyl-1-hexyl, 3-methyl-1-hexyl, 2,2-dimethyl-1-pentyl, 2,3-dimethyl-1-pentyl, 2,4-dimethyl-1-pentyl, 3,3-dimethyl-1-pentyl, 2,2,3-trimethyl-1-butyl, 3-ethyl-1-pentyl, 1-octyl (n-octyl), 1-nonyl (n-nonyl), 1-decyl (n-decyl), etc.

By the terms propyl, butyl, pentyl, hexyl, heptyl, octyl, nonyl, decyl, etc. without any further definition are meant saturated hydrocarbon groups with the corresponding number of carbon atoms, wherein all isomeric forms are included.

The above definition for alkyl also applies if alkyl is a part of another (combined) group such as for example C_{x-y} alkylamino or C_{x-y} alkoxy.

Unlike alkyl, alkenyl, when used alone or in combination herein, consists of at least two carbon atoms, wherein at least two adjacent carbon atoms are joined together by a C═C double bond and a carbon atom can only be part of one C═C double bond. If in an alkyl as hereinbefore defined having at least two carbon atoms, two hydrogen atoms on adjacent carbon atoms are formally removed and the free valencies are saturated to form a second bond, the corresponding alkenyl is formed. Alkenyl may optionally be present in the cis or trans or E or Z orientation with regard to the double bond(s).

Unlike alkyl, alkynyl, when used alone or in combination herein, consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Haloalkyl (haloalkenyl, haloalkynyl), when used alone or in combination herein, is derived from the previously defined alkyl (alkenyl, alkynyl) by replacing one or more hydrogen atoms of the hydrocarbon chain independently of one another by halogen atoms, which may be identical or different. If a haloalkyl (haloalkenyl, haloalkynyl) is to be further substituted, the substitutions may take place independently of one another, in the form of mono- or polysubstitutions in each case, on all the hydrogen-carrying carbon atoms. Examples of haloalkyl (haloalkenyl, haloalkynyl) are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF=CF₂, —CCl=CH₂, —CBr=CH₂, —C—C—CF₃, —CHFCH₂CH₃, —CHFCH₂CF₃, etc.

The term “aryl,” when used alone or in combination herein, refers to an aromatic hydrocarbon ring containing from six to fourteen carbon ring atoms (e.g., a C₆₋₁₄ aryl, preferably C₆₋₁₀ aryl). The term C₆₋₁₄ aryl includes monocyclic rings, fused rings and bicyclic rings where at least one of the rings is aromatic. Non-limiting examples of C₆₋₁₄ aryls include phenyl, indanyl, indenyl, benzocyclobutanyl, dihydronaphthyl, tetrahydronaphthyl, naphthyl, benzocycloheptanyl, and benzocycloheptenyl.

As used herein, the term “heteroaryl,” when used alone or in combination herein, refers to a heteroaromatic ring system that contains 2-10 carbon atoms and 1-4 heteroatom ring atoms selected from N, NH, NR′, O, and S wherein R′ is C₁₋₆-alkyl and includes aromatic 5 to 6-membered monocyclic heteroaryls and aromatic 7 to 11-membered heteroaryl bicyclic or fused rings where at least one of the rings is aromatic. Non-limiting examples of 5 to 6-membered monocyclic heteroaryl rings include furanyl, oxazolyl, isoxazolyl, oxadiazolyl, thiazolyl, pyrazolyl, pyrrolyl, imidazolyl, tetrazolyl, triazolyl, thienyl, thiadiazolyl, pyridinyl, pyrimidinyl, pyridazinyl, pyrazinyl, triazinyl, and 1,3-dihydrobenzoimidazol-2-one. Non-limiting examples of 7 to 11-membered heteroaryl bicyclic or fused rings include purinyl, benzimidazolyl, quinolinyl, dihydro-2H-quinolinyl, isoquinolinyl, quinazolinyl, indazolyl, thieno[2,3-d]pyrimidinyl, indolyl, isoindolyl, indazolyl, benzotriazolyl, benzofuranyl, benzopyranyl, benzodioxolyl, benzoxazolyl, benzothiazolyl, pyrrolo[2,3-b]pyridinyl, and imidazo[4,5-b]pyridinyl. Sulfur and nitrogen may optionally be present in all the possible oxidation stages (sulphur: sulphoxide —SO—, sulphone —SO₂—; nitrogen: N-oxide).

The compounds of the invention are only those which are contemplated to be chemically stable as will be appreciated by those skilled in the art. For example, a compound which would have a “dangling valency,” or a carbanion are not compounds contemplated by the inventive methods disclosed herein.

“Alkoxy” as used herein refers to an alkyl-O— group wherein the alkyl group is as defined before. The term “C_{x-y}-alkoxy” (x and y each being an integer) as used herein refers to an alkoxy group as defined before containing x to y carbon atoms. For example, a C₁₋₆-alkoxy group means a group of the formula C₁₋₆-alkyl-O— in which the term “C₁₋₆-alkyl” has the previously given significance. Examples of alkoxy groups are methoxy and ethoxy.

“Halogen” as used herein refers to F, Cl, Br, or I.

The term “cycloalkyl,” when used alone or in combination herein, refers to a nonaromatic 3 to 12-membered (but preferably, 3 to 6-membered) monocyclic carbocyclic radical or a nonaromatic 6 to 10-membered fused bicyclic, bridged bicyclic, propellane, or spirocyclic carbocyclic radical. The C₃₋₁₂ cycloalkyl may be either saturated or partially unsaturated, and the carbocycle may be attached by any atom of the cycle which results in the creation of a stable structure. Non-limiting examples of 3 to 10-membered monocyclic carbocycles include cyclopropyl, cyclobutyl, cyclopentyl, cyclopentenyl, cyclohexyl, cyclohexenyl, cycloheptanyl, cycloheptenyl, and cyclohexanone. Non-limiting examples of 6 to 10-membered fused bicyclic carbocyclic radicals include bicyclo[3.3.0]octane, bicyclo[4.3.0]nonane, and bicyclo[4.4.0]decanyl (decahydronaphthalenyl). Non-limiting examples of 6 to 10-membered bridged bicyclic carbocyclic radicals include bicyclo[1.1.1]pentane, bicyclo[2.2.2]heptanyl, bicyclo[2.2.2]octanyl, and bicyclo[3.2.1]octanyl. Non-limiting examples of 6 to 10-membered propellane carbocyclic radicals include but are not limited to [1.1.1]propellane, [3.3.3]propellane, and [3.3.1]propellane. Non-limiting examples of 6 to 10-membered spirocyclic carbocyclic radicals include but are not limited to spiro[3,3]heptanyl, spiro[3,4]octanyl, and spiro[4,4]heptanyl.

The term “heterocyclyl,” when used alone or in combination herein, refers to a heterocyclic ring system that contains 2-10 carbon atoms and one to four heteroatom ring atoms chosen from NH, NR′, oxygen, and sulfur wherein R′ is C₁₋₆ alkyl and includes stable nonaromatic 4-8 membered monocyclic heterocyclic radical or a stable nonaromatic 6 to 11-membered fused bicyclic, bridged bicyclic or spirocyclic heterocyclic radical. The heterocycle may be either completely saturated or partially unsaturated. In one embodiment, the heterocycle is a C₃₋₆ heterocycle, i.e., containing 3 to 6 ring carbon atoms. Non-limiting examples of nonaromatic monocyclic heterocyclic radicals include tetrahydrofuranyl, azetidinyl, pyrrolidinyl, pyranyl, tetrahydropyranyl, dioxanyl, thiomorpholinyl, 1,1-dioxo-1.lambda₆-thiomorpholinyl, morpholinyl, piperidinyl, piperazinyl, and azepinyl. Non-limiting examples of nonaromatic 6 to 11-membered fused bicyclic heterocyclic radicals include octahydroindolyl, octahydrobenzofuranyl, and octahydrobenzothiophenyl. Non-limiting examples of nonaromatic 6 to 11-membered bridged bicyclic radicals include 2-azabicyclo[2.2.1]heptanyl, 3-azabicyclo[3.1.0]hexanyl, and 3-azabicyclo[3.2.1]octanyl. Non-limiting examples of nonaromatic 6 to 11-membered spirocyclic heterocyclic radicals include 7-aza-spiro[3,3]heptanyl, 7-spiro[3,4]octanyl, and 7-aza-spiro[3,4]octanyl.

Sulfur and nitrogen may optionally be present in all the possible oxidation stages (sulphur 4 sulphoxide —SO—, sulphone —SO₂—; nitrogen 4 N-oxide).

“Hydroxy” as used herein refers to —OH.

“Carboxy” as used herein refers to —COOH.

“Compound(s) of the Invention” or “compound(s) of the invention” encompass compounds disclosed herein, e.g., any of Formula I or 1.1-1.37, in free or salt form (e.g., pharmaceutically acceptable salt form).

Compounds disclosed herein, e.g., any of Formula I or 1.1-1.37, may exist in free or salt form, e.g., as acid addition salts. In this patent application, unless otherwise indicated, language such as “Compound(s) of the Invention,” “compound(s) of the invention,” “compounds disclosed herein, e.g., any of Formula I or 1.1-1.37,” is to be understood as embracing the compounds in any form, for example free or acid addition salt form (e.g., pharmaceutically acceptable acid addition salt form), or where the compounds contain acidic substituents, in base addition salt form (e.g., pharmaceutically acceptable base addition salt form). Any reference to “Compounds of the Invention,” “compounds of the invention,” and compound(s) of Formula I, e.g., any of Formula I or 1.1-1.37, is to be understood as referring also to the salts (and especially the pharmaceutically acceptable salts) of such compounds, as appropriate and expedient. Compounds disclosed herein are intended for use as pharmaceuticals, therefore pharmaceutically acceptable salts are preferred. Salts that are unsuitable for pharmaceutical uses may be useful, for example, for the isolation or purification of free forms of compounds disclosed herein or their pharmaceutically acceptable salts, so therefore are also included.

“Pharmaceutically acceptable salt” as used herein refers to salts that retain the desired biological activity of the subject compound and exhibit minimal undesired toxicological effects. Such salts include inorganic or organic acid and/or base addition salts depending on the presence of basic and/or acidic groups in the subject compound.

Some compounds disclosed herein may contain one or more asymmetric centers. This patent application provides the use of any of the optically pure stereoisomers as well as any combination of stereoisomers. Accordingly, compounds disclosed herein, e.g., any of Formula I or 1.1-1.37, in free or salt (e.g., pharmaceutically acceptable salt) form, include their enantiomers, diastereoisomers, stereoisomers, and racemates. Mixtures of stereoisomers may be separated in a manner known to a person skilled in the art.

Compounds disclosed herein may also contain one or more double bonds. Representations of double bonds in this invention are meant to include both the E and the Z isomer of the double bond.

Compounds disclosed herein, e.g., any of Formula I or 1.1-1.37, in free or salt (e.g., pharmaceutically acceptable salt) form, also encompass their stable and unstable isotopes. Stable isotopes are nonradioactive isotopes that contain one additional neutron compared to the abundant nuclides of the same species (i.e., element). For example, the hydrogen atom at a certain position on a compound disclosed herein, e.g., any of Formula I or 1.1-1.37, in free or salt (e.g., pharmaceutically acceptable salt) form, may be replaced with deuterium (a stable isotope which is non-radioactive). Examples of known stable isotopes include, but are not limited to, deuterium, ¹³C, ¹⁵N, ¹⁸O. Alternatively, unstable isotopes, which are radioactive isotopes that contain additional neutrons compared to the abundant nuclides of the same species (i.e., element), e.g., ¹²³I, ¹³¹I, ¹²⁵I, ¹¹C, ¹⁸F, may replace the corresponding abundant species of I, C, and F. Another example of a useful isotope of the compound of the invention is the ¹¹C isotope. These radio isotopes are useful for radio-imaging and/or pharmacokinetic studies of compounds disclosed herein, e.g., any of Formula I or 1.1-1.37, in free or salt (e.g., pharmaceutically acceptable salt) form.

In this patent application, a bond drawn with a wavy line shows the point of attachment of the radical drawn.

Further provided is a pharmaceutical composition comprising a compound disclosed herein, e.g., any of Formula I or 1.1-1.37, in free or pharmaceutically acceptable salt form. For instance, provided is a pharmaceutical composition comprising a compound as disclosed herein, e.g., any of Formula I or 1.1-1.37, in free or pharmaceutically acceptable salt form, in admixture with a pharmaceutically acceptable carrier.

Further provided are methods of using the compounds and pharmaceutical compositions disclosed herein, e.g., any of Formula I or 1.1-1.37, in free or pharmaceutically acceptable salt form, for treatment or prophylaxis of diseases and disorders as set forth herein. For instance, for treatment of TYK2-mediated disorders in a patient (e.g., a human) in need thereof, such as, autoimmune and inflammatory disorders (includes any disease having an inflammatory or autoimmune component), metabolic disorders, proliferative disorders, endocrine disorders, neurological disorders, allergic disorders, cancer, or a disorder associated with transplantation. Or, for instance, for treatment of disease that benefits from modulation of IL-12, IL-23, and/or IFNα.

Further provided is a method for modulation of IL-12, IL-23, and/or IFNα by inhibiting TYK2 mediated signal transduction in a patient (e.g., a human) in need thereof, wherein the method comprises administering an effective amount of a compound (including a pharmaceutical composition comprising an effective amount of a compound) disclosed herein, e.g., any of Formula I or 1.1-1.37, in free or pharmaceutically acceptable salt form.

Further provided is a method of prophylaxis or treatment of any one or more of the following conditions in a patient (e.g., a human) in need thereof:
(i) a TYK2-mediated disorder, for instance, TYK2-mediated autoimmune or inflammatory disorders (includes any disease having an inflammatory or autoimmune component), metabolic disorders, proliferative disorders, endocrine disorders, neurological disorders, allergic disorders, cancer, and/or a disorder associated with transplantation; and/or
(ii) an autoimmune disorder, an inflammatory disorder, a metabolic disorder, a proliferative disorder, an endocrine disorder, a neurological disorder, an allergic disorder, cancer, and/or a disorder associated with transplantation; and/or
(iii) rheumatoid arthritis, multiple sclerosis, systemic lupus erythematosus (SLE), lupus nephritis, cutaneous lupus, inflammatory bowel disease, psoriasis, Crohn's Disease, psoriatic arthritis, Sjogren's syndrome, systemic scleroderma, ulcerative colitis, Graves' disease, discoid lupus erythematosus, adult onset Stills, systemic onset juvenile idiopathic arthritis, gout, gouty arthritis, type 1 diabetes, insulin dependent diabetes mellitus, sepsis, septic shock, Shigellosis, pancreatitis (acute or chronic), glomerulonephritis, autoimmune gastritis, diabetes, autoimmune hemolytic anemia, autoimmune neutropenia, thrombocytopenia, atopic dermatitis, myasthenia gravis, pancreatitis (acute or chronic), ankylosing spondylitis, pemphigus vulgaris, Goodpasture's disease, antiphospholipid syndrome, idiopathic thrombocytopenia, ANCA-associated vasculitis, pemphigus, Kawasaki disease, Chronic Inflammatory Demyelinating Polyneuropathy (CIDP), dermatomyositis, polymyositis, uveitis, Guillain-Barre syndrome, autoimmune pulmonary inflammation, autoimmune thyroiditis, autoimmune inflammatory eye disease, and/or chronic demyelinating polyneuropathy; and/or
(iv) systemic lupus erythematosus (SLE), lupus nephritis, cutaneous lupus, Crohn's Disease, ulcerative colitis, type 1 diabetes, psoriasis, rheumatoid arthritis, systemic onset juvenile idiopathic arthritis, ankylosing spondylitis, and/or multiple sclerosis; and/or
(v) rheumatoid arthritis; and/or
(vi) acute myelogenous leukemia, chronic myelogenous leukemia, metastatic melanoma, Kaposi's sarcoma, multiple myeloma, solid tumors, ocular neovasculization, infantile haemangiomas, B cell lymphoma, systemic lupus erythematosus (SLE), rheumatoid arthritis, psoriatic arthritis, multiple vasculitides, idiopathic thrombocytopenic purpura (ITP), myasthenia gravis, allergic rhinitis, multiple sclerosis (MS), transplant rejection, Type I diabetes, membranous nephritis, inflammatory bowel disease, autoimmune hemolytic anemia, autoimmune thyroiditis, cold and warm agglutinin diseases, Evans syndrome, hemolytic uremic syndrome/thrombotic, thrombocytopenic purpura (HUS/TTP), sarcoidosis, Sjogren's syndrome, peripheral neuropathies, pemphigus vulgaris, and/or asthma; and/or
(vii) an inflammatory disease such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosis, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, and/or gouty arthritis; and/or
(viii) metabolic diseases including type 2 diabetes, atherosclerosis, myocardial infarction; destructive bone disorders such as bone resorption disease, osteoarthritis, osteoporosis, and/or multiple myeloma-related bone disorder; and/or
(ix) proliferative disorders such as acute myelogenous leukemia, chronic myelogenous leukemia; angiogenic disorders such as angiogenic disorders including solid tumors, ocular neovasculization and infantile haemangiomas; infectious diseases such as sepsis, septic shock, and/or Shigellosis; and/or
(x) neurodegenerative diseases such as Alzheimer's disease, Parkinson's disease, cerebral ischemias or neurodegenerative disease caused by traumatic injury, oncologic and viral diseases such as metastatic melanoma, Kaposi's sarcoma, multiple myeloma, and HIV infection and CMV retinitis, and/or AIDS; and/or
(xi) ulcerative colitis (e.g., moderate to severe ulcerative colitis); and/or
(xii) Crohn's Disease; and/or
(xiii) psoriasis (e.g., nail psoriasis and/or plaque psoriasis (e.g., moderate to severe plaque psoriasis); and/or
(xiv) psoriatic arthritis and/or
(xv) systemic lupus erythematosus;
   wherein the method comprises administering an effective amount of a compound disclosed herein (including a pharmaceutical composition comprising an effective amount of a compound), e.g., any of Formula I or 1.1-1.37, in free or pharmaceutically acceptable salt form, to the patient.

For instance, provided is a method of prophylaxis or treatment of any one or more of the following conditions in a patient (e.g., a human) in need thereof: pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft versus host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic β-cell disease, diseases characterized by massive neutrophil infiltration, rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, Shigellosis, Alzheimer's disease, Parkinson's disease, cerebral ischemia or neurodegenerative disease caused by traumatic injury, angiogenic disorders including solid tumors, ocular neovasculization, and infantile haemangiomas, viral diseases including acute hepatitis infection (including hepatitis A, hepatitis B and hepatitis C), HIV infection and CMV retinitis, AIDS, ARC or malignancy, herpes, stroke, myocardial ischemia, ischemia in stroke, heart attacks, organ hypoxia, vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris, wherein the method comprises administering an effective amount of a compound disclosed herein (including a pharmaceutical composition comprising an effective amount of a compound), e.g., any of Formula I or 1.1-1.37, in free or pharmaceutically acceptable salt form, to the patient. Preferred methods of treatment are those wherein the condition is selected from Crohn's disease, ulcerative colitis, allograft rejection, rheumatoid arthritis, psoriasis, ankylosing spondylitis, psoriatic arthritis, and pemphigus vulgaris, wherein the method comprises administering an effective amount of a compound disclosed herein (including a pharmaceutical composition comprising an effective amount of a compound), e.g., any of Formula I or 1.1-1.37, in free or pharmaceutically acceptable salt form, to the patient.

Also provided is a method of prophylaxis or treatment of ischemia reperfusion injury, including cerebral ischemia reperfusion injury arising from stroke and cardiac ischemia reperfusion injury arising from myocardial infarction in a patient (e.g., a human) in need thereof, wherein the method comprises administering an effective amount of a compound (including a pharmaceutical composition comprising an effective amount of a compound) disclosed herein, e.g., any of Formula I or 1.1-1.37, in free or pharmaceutically acceptable salt form, to the patient.

Further provided is a method of inhibiting a kinase activity (e.g., TYK2 enzyme activity) in a patient (e.g., a human) in need thereof or in a biological sample (e.g., cell or tissue), wherein the method comprises contacting the patient or biological sample with a compound disclosed herein (including a pharmaceutical composition comprising an effective amount of a compound), e.g., any of Formula I or 1.1-1.37, in free or pharmaceutically acceptable salt form.

Further provided is:
(i) a compound disclosed herein, e.g., any of Formula I or 1.1-1.37, in free or pharmaceutically acceptable salt form, for use in any method or in the treatment or prophylaxis of any disease or condition set forth herein;
(ii) the use of a compound disclosed herein, e.g., any of Formula I or 1.1-1.37, in free or pharmaceutically acceptable salt form, for treatment or prophylaxis of any disease or condition set forth herein;
(iii) the use of a compound disclosed herein, e.g., any of Formula I or 1.1-1.37, in free or pharmaceutically acceptable salt form, in the manufacture of a medicament for treatment or prophylaxis of any disease or condition set forth herein;
(iv) a pharmaceutical composition comprising a compound disclosed herein, e.g., any of Formula I or 1.1-1.37, in free or pharmaceutically acceptable salt form, in combination or association with a pharmaceutically acceptable diluent or carrier; and
(v) a pharmaceutical composition comprising a compound disclosed herein, e.g., any of Formula I or 1.1-1.37, in free or pharmaceutically acceptable salt form, in combination or association with a pharmaceutically acceptable diluent or carrier for use in the treatment or prophylaxis of any disease or condition set forth herein.

In some embodiments, compounds disclosed herein, e.g., any of Formula I or 1.1-1.37, in free or pharmaceutically acceptable salt form, may be used in the foregoing methods of treatment or prophylaxis as a sole therapeutic agent, but may also be used in combination or for co-administration with other active agents. For co-administration, the compound disclosed herein, e.g., any of Formula I or 1.1-1.37, in free or pharmaceutically acceptable salt form, may be administered sequentially (e.g., contemporaneously or as part of the same treatment regimen) or simultaneously with the second active agent. Where two active agents are administered, the effective amount of each agent may be below the amount needed for activity as monotherapy. Accordingly, a subthreshold amount (i.e., an amount below the level necessary for efficacy as monotherapy) may be considered therapeutically effective and may also be referred alternatively as a therapeutically effective amount. Indeed, an advantage of administering different agents with different mechanisms of action and different side effect profiles may be to reduce the dosage and side effects of either or both agents, as well as to enhance or potentiate their activity as monotherapy.

For methods of treatment, “effective amount” as used herein is intended to encompass a therapeutically effective amount to treat a specific disease or disorder.

“Patient” as used herein includes human and non-human (i.e., animal).

“Comprising” as used herein is intended to be open-ended and does not exclude additional, unrecited elements or method steps.

Dosages employed in practicing the methods disclosed herein will of course vary depending, e.g., on the patient, on the particular disease or condition to be treated, the particular compound used, the mode of administration, and the therapy desired. Compounds disclosed herein, e.g., any of Formula I or 1.1-1.37, in free or pharmaceutically acceptable salt form, may be administered by any suitable route, including orally, parenterally, transdermally, or by inhalation, but are preferably administered orally.

Compounds disclosed herein, e.g., any of Formula I or 1.1-1.37, in free or salt (e.g., pharmaceutically acceptable salt) form, may be made using the methods as described and exemplified herein and by methods similar thereto and by methods known in the chemical art. Such methods include, but are not limited to, those described below. If not commercially available, starting materials for these processes may be made by procedures, which are selected from the chemical art using techniques which are similar or analogous to the synthesis of known compounds.

Accordingly, also provided are methods of making the compounds and pharmaceutical compositions disclosed herein, e.g., any of Formula I or 1.1-1.37, in free or salt (e.g., pharmaceutically acceptable salt) form. For instance, provided is a method (Method 1) for a making a compound disclosed herein, e.g., any of Formula I or 1.1-1.37, in free or salt form (e.g., pharmaceutically acceptable salt form), as set forth in. Salts that are unsuitable for pharmaceutical uses may be useful, for example, for the isolation or purification of free forms of compounds disclosed herein or their pharmaceutically acceptable salts, so therefore are also included.

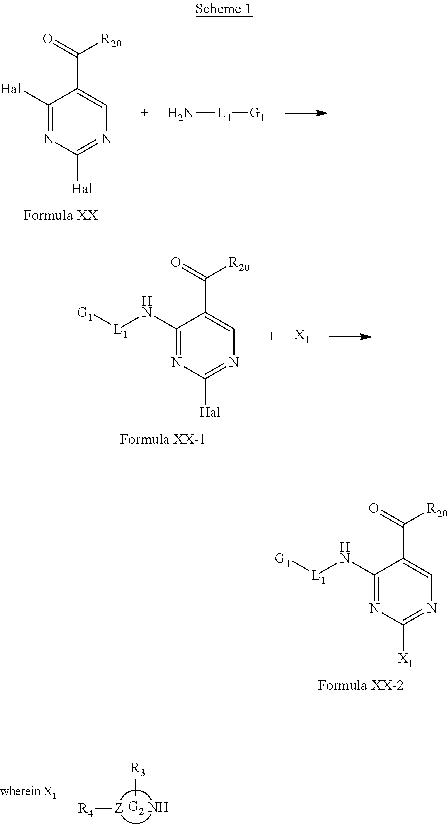

In Scheme 1, Hal is a halogen (e.g., Cl), R₂₀ is —OCH₂CH₃ or NHR₁, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, R₂ is a 5- to 6-membered saturated heterocyclyl, G₂ is a 4- to 12-membered saturated heterocyclyl, Z is CH or N, R₃ is H or C₁₋₃-alkyl, R₄ is H or —C(O)R₅, and R₅ is C₁₋₃-alkyl, OH, NH₂, —NHC₁₋₃-alkyl, C₁₋₆-alkoxy, or C₃₋₆-cycloalkyl.

In Scheme 1, Formula XX-1 may be prepared by reacting Formula XX and H₂N-L₁-G₁ via a heteroaryl amination or coupling reaction as described below. Formula XX-2 may also be prepared by reacting Formula XX-1 and X1 via a heteroaryl amination or coupling reaction as described below. Further modification of substituents in Formula XX-1 and Formula XX-2 may be achieved by sulfur oxidation and saponification reactions as described below. Some reactions may require a substituent to be protected by a protecting group (PG). The use of protecting groups is well known in the art (see for example “Protective Groups in Organic Synthesis,” T. W. Greene, P. G. M. Wuts, Wiley-Interscience, 1999).

Pharmaceutical compositions comprising a compound disclosed herein, e.g., any of Formula I or 1.1-1.37, in free or pharmaceutically acceptable salt form, may be prepared using conventional diluents or excipients and techniques known in the galenic art. Thus oral dosage forms may include tablets, capsules, solutions, suspensions and the like.

### EXAMPLES

The synthetic methods for various compounds disclosed herein, e.g., any of Formula I or 1.1-1.37, in free or salt (e.g., pharmaceutically acceptable salt) form, are illustrated below. The intermediates of compounds disclosed herein, e.g., any of Formula I or 1.1-1.37, in free or salt (e.g., pharmaceutically acceptable salt) form, as well as other compounds disclosed herein and their salts (e.g., pharmaceutically acceptable salts), may be made using the methods as similarly described below and/or by methods similar to those generally described in the detailed description and by methods known in the chemical art.

### Abbreviations

aq. Aqueous
Boc/BOC/t-Boc tert-butyloxycarbonyl
DCM Dichloromethane
DIPEA Diisopropylethylamine
DME 1,2-dimethoxyethane
DMSO Dimethylsulfoxide
equiv. or eq Equivalent(s)
Et₃N/TEA Triethylamine
EtOAc Ethyl acetate
EtOH Ethanol
h Hour(s)
HATU 1-[Bis(dimethylamino)methylene]-1H-1,2,3-triazolo[4,5-b]pyridinium 3-oxide hexafluorophosphate
MeOH Methanol
min Minute(s)
MPLC Medium pressure liquid chromatography
MS Mass spectrometry
N Normality
NMR Nuclear magnetic resonance spectrometry
Rf Retention factor
RT/rt Room temperature
THF Tetrahydrofuran
TLC Thin layer chromatography
Pd(dppf)Cl₂ 1,1′-Bis(diphenylphosphino)ferrocene]palladium (II) dichloride
Pd(dtbpf)Cl₂ [1,1′-Bis(di-tert-butylphosphino) ferrocene]dichloropalladium(II)
NH₄Cl Ammonium Chloride
LiHMDS Lithium hexamethyldisilazide
NaHCO₃ Sodium bicarbonate
Na₂SO₄ Sodium Sulphate
Na₂CO₃ Sodium carbonate
Pd/C Palladium on carbon
Pd(OH)₂ Palladium hydroxide
CH₃CN/ACN Acetonitrile
Fe(acac)₃ Tris(acetylacetonato) iron (III)
NMP N-Methyl-2-pyrrolidone
i-prMgCl Isopropylmagnesium Chloride
RM Reaction mixture
MsCl Mesyl chloride
DMF Dimethyl formamide
K₃PO₄ Potassium phosphate tribasic
K₂CO₃ Potassium carbonate
(Boc)₂O Di-tert-butyl dicarbonate
TBAF Tetra-n-butylammonium fluoride
NaH Sodium hydride
t-BuOH tert-Butyl alcohol
Py Pyridine
NH₄₀H Ammonium hydroxide
DMAP 4-Dimethylaminopyridine
Na₂CO₃ Sodium carbonate
t-BuLi tert-Butyllithium
B(OⁱPr)₃ Triisopropyl borate
Et₂O Diethyl ether
IPA 2-Propanol
NCS N-Chloro succinimide
CsCO₃ Caesium carbonate
H₂ Hydrogen
MW Microwave
NaBH₄ Sodium borohydride
NH₂OH·HCl Hydroxylammonium chloride
HCl Hydrochloric Acid
Xantphos 4,5-Bis(diphenylphosphino)-9,9-dimethylxanthene
T3P propanephosphonic anhydride or propylphosphonic anhydride
EDC 1-ethyl-3-(3-dimethylaminopropyl)carbodiimide
HOBt 1-hydroxybenzotriazole

### LCMS Methods:

Method A: Column: Kinetex C18, 4.6×50 mm, 2.6 μm, 100 A; Mobile Phase A:0.1% Formic Acid in Aq., B: ACN; T/% B: 0/5, 0.5/5, 2/95, 3.2/95, 3.21/5, 4/5; Flow rate: 1.5 ml/min; Instrument: Agilent 6120 Quadrupole LCMS.
Method B: Kinetex C18, 4.6×30 mm, 2.6 μm, 100 A; Mobile Phase: A 0.1% Formic Acid in water, B: ACN; Method-T/% B-0/5, 0.5/95, 2.4/95, 2.5/5, 3.5/5; Flow rate 1.5 ml/Min; Instrument SHIMADZU 2020 LCMS;
Method C: Column: Kinetex C18, 4.6×50 mm, 2.6 μm, 100 A Mobile Phase A:0.1% Formic Acid in Aqs, B: ACN T/% B: 0/5, 0.5/5, 2/95, 3.2/95, 3.21/5, 4/5; Flow rate: 1.5 ml/min Instrument: Agilent 6120 Quadrupole LCMS.
Method D: Column: Kinetex EVO C-18 (150*4.6)mm 5 μm, 100 A Mobile phase-A: 0.1% Formic Acid in Aq; Mobile phase-B: ACN 100% Method-T/% B: 0/2, 2/2, 6/90, 10/90, 11/2, 12/2 Flow rate: 1.0 ml/min Column temp: 30° C. Diluent: ACN+H2O
Method-E: Column:Kinetex EVO C18 (150*4.6) mm*5 μm, 100 A Mobile phase-A:0.1% Formic Acid in (Aq); Mobile phase-B:ACN 100% Method-T/% B:-0/2, 2/2, 6/85, 10/85, 11/2, 12/2 Flow rate: 1.0 ml/min Column temp: 30° C. Diluent: ACN+H2O

### HPLC Methods:

Method A: Column: Kinetex EVO C-18 (150*4.6) mm, 5 μm, 100 A; Mobile phase-A: 0.01M Ammonium Acetate in (Aq.); Mobile phase-B: ACN 100%; Method-T/% B:-0/10, 2/10, 6/95, 10/95, 11/10, 12/10; Flow rate: 1.0 ml/min; Column temp: 30° C.
Method B: Column: Kinetex EVO C-18 (150*4.6) mm 5 μm, 100 A Mobile phase-A: 0.1% Formic Acid in Aq; Mobile phase-B:ACN 100% Method-T/% B: 0/10, 2/10, 6/90, 10/90, 11/10, 12/10 Flow rate: 1.0 ml/min Column temp: 30° C.
Method C: Column: Kinetex EVO C-18 (150*4.6) mm 5 μm, 100 A; Mobile phase-A: 0.1% Formic Acid in Aq.; Mobile phase-B:ACN 100%. Method-T/% B: 0/2, 2/2, 6/80, 10/80, 11/2, 12/2; Flow rate: 1.0 ml/min; Column temp: 30° C.
Method D: Column: Kinetex EVO C18(150*4.6) mm*5 μm, 100 A; Mobile phase-A:0.1% Formic Acid in (Aq.); Mobile phase-B:ACN 100%; Method-T/% B:-0/10, 2/10, 6/95, 10/95, 11/10, 12/10; Flow rate: 1.0 ml/min; Column temp: 30° C.
Method E: Column: YMC Triart EXRS C-18 (150*4.6)mm, 5 μm, Mobile phase-A: 0.1% TFA in Aq; Mobile phase-B:ACN 100%, Method-T/% B:0/10, 2/10, 6/95, 10/95, 11/10, 12/10, Flow rate: 1.0 ml/min, Column temp: 30° C., Diluent: ACN+H2O.
Method F: Method G: Column: Kinetex EVO C18 (150*4.6) mm*5,100 A Mobile phase-A:0.1% Formic Acid in (Aq); Mobile phase-B: ACN 100%, Method-T/% B: 0/2, 2/2, 6/85, 10/85, 11/2, 12/2, Flow rate: 1.0 ml/min, Column temp: 30° C., Diluent: ACN+H2O.

### Representative Preparations of Precursors and Intermediates

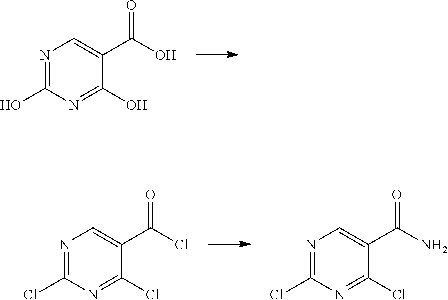 [SMILES: O=C(O)c1cnc(O)nc1O ; O=C(Cl)c1cnc(Cl)nc1Cl ; NC(=O)c1cnc(Cl)nc1Cl]

### 2,4-dichloropyrimidine-5-carbonyl Chloride

To a stirred solution of 2,4-dihydroxypyrimidine-5-carboxylic acid (7.50 g, 48.046 mmol, 1.0 eq) in POCl₃ (34 mL) at 0° C. is added PCl₅ (34.01 g, 163.35 mmol, 3.4 eq) and the reaction mixture is stirred at 110° C. for 16 h. Completion of the reaction is monitored by TLC. After completion of the reaction, POCl₃ is distilled out completely under reduced pressure and co-distilled with toluene (100 ml×2) and dried under reduced pressure. The crude material is taken to next step as such, as yellow gummy solid (7.50 g, crude). 2,4-dichloropyrimidine-5-carboxamide

To a stirred solution of 2,4-dichloropyrimidine-5-carbonyl chloride (7.5 g, 35.72 mmol, 1.0 eq) in THF (30 mL) at 0° C. is added NH₄OH (7.5 ml) slowly dropwise. The mixture is stirred at RT for 1 h. Completion of the reaction is monitored by TLC. After completion, the reaction mixture is quenched by chilled water (20 ml) and the compound extracted with ethyl acetate (200 ml×2), dried over Na₂SO₄ and the solvent evaporated under reduced pressure. The crude compound is purified by MPLC using MeOH/DCM (gradient 1-2% MeOH) to afford the desired compound as an off white solid (3.00 g, 43% yield). LCMS (Method B): m/z=190.95 (M−H)⁺.

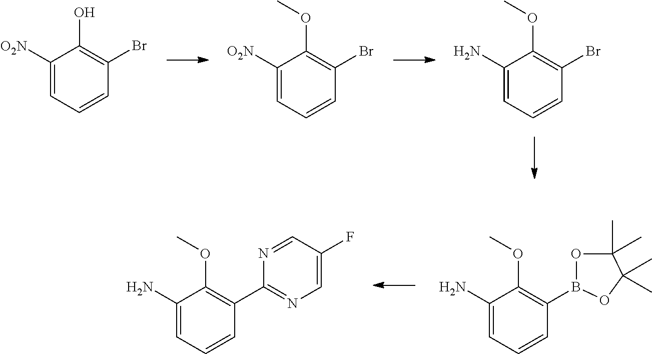

### 1-bromo-2-methoxy-3-nitrobenzene

To a stirred solution of 2-bromo-6-nitrophenol (4.0 g, 18.34 mmol, 1.0 eq) in DMF (32 mL) in a 250 mL sealed tube under argon atmosphere are added K₂CO₃ (7.59 g, 55.04 mmol, 3.0 eq) and iodomethane (2.28 mL, 36.69 mmol, 2.0 eq) at RT. The reaction tube is sealed and stirred at RT for 16 h. The progress of the reaction is monitored by TLC. The reaction mixture is poured into ice cold water (200 ml), the obtained solid is filtered and dried completely to afford the desired compound as an off-white solid (3.8 g, 90% yield).

### 3-bromo-2-methoxyaniline

To a stirred solution of 1-bromo-2-methoxy-3-nitrobenzene (3.8 g, 16.45 mmol, 1.0 eq) in MeOH:H₂O (3:1) (100 mL) are added ammonium chloride (2.64 g, 49.3 mmol, 3.0 eq) and zinc (4.3 g, 65.81 mmol, 4.0 eq) at RT. The reaction mixture is stirred at RT for 16 h. The progress of the reaction is monitored by TLC. The reaction mixture is filtered through celite pad and washed with MeOH (50 mL) and the filtrate is concentrated to dryness. The residue is dissolved in ethyl acetate (300 mL), washed with water (50 mL) and brine (30 mL), dried over anhydrous Na₂SO₄ and concentrated and dried to afford the desired compound as an off-white solid (3.0 g, 90% yield).

### 2-methoxy-3-(4,4,5,5-tetramethyl-1,3,2-dioxaborolan-2-yl)aniline

To a stirred solution of KOAc (2.92 g, 29.8 mmol, 2.0 eq) in 1,4-dioxane (50 mL) under argon atmosphere are added 3-bromo-2-methoxyaniline (3.0 g, 14.9 mmol, 1.0 eq) and 4,4,4′,4′,5,5,5′,5′-octamethyl-2,2′-bi(1,3,2-dioxaborolane) (4.54 g, 17.9 mmol, 1.2 eq) at RT. The reaction mixture is stirred under argon atmosphere for 10 min and then PdCl₂(dppf).DCM complex (609 mg, 0.74 mmol, 0.05 eq) is added. The reaction mixture is heated at 90° C. for 16 h. The progress of the reaction is monitored by TLC. The reaction mixture is diluted with water (50 mL) and extracted with ethyl acetate (2×100 mL). The combined organic layers are washed with water (30 mL) and brine (30 mL), dried over Na₂SO₄ and concentrated to dryness. The crude compound is purified by flash chromatography. The product is eluted with 35% ethyl acetate in hexanes. The fractions containing the product are concentrated under reduced pressure and dried completely to afford the desired compound as a pale brown semi solid (3.2 g, 86% yield). LCMS (Method B): m/z=250.10 (M+H)⁺ (tRet: 1.36 min).

### 3-(5-fluoropyrimidin-2-yl)-2-methoxyaniline

Note: This reaction is performed in 3×500 mg batches. The weight/volume of chemicals and solvent are divided by 3.

To a stirred solution of 2M aq. K₃PO₄ (2.55 g (6 mL), 12.04 mmol, 2.0 eq) in dioxane (9 mL) in 30 mL microwave tube under argon atmosphere are added a solution of 2-methoxy-3-(4,4,5,5-tetramethyl-1,3,2-dioxaborolan-2-yl)aniline (1.5 g, 6.02 mmol, 1.0 eq) in dioxane (3 mL), 2-bromo-5-fluoropyrimidine (1.08 g, 6.02 mmol, 1.0 eq) and Pd(dppf)Cl₂·DCM complex (245.82 mg, 0.30 mmol, 0.05 eq) at RT. The reaction tube is sealed and placed in microwave apparatus and applied temperature 110° C. for 1.5 h. The progress of the reaction is monitored by TLC. The reaction mixture is diluted with water (30 ml) and extracted with ethyl acetate (2×150 ml). The combined organic layer is washed with brine (30 ml), dried over anhydrous Na₂SO₄ and concentrated to dryness to afford the crude compound. The crude compound is purified by flash chromatography. The product is eluted with 20% ethyl acetate in hexanes. The fractions containing the product are concentrated under reduced pressure and dried completely to afford the desired compound as a pale brown oil or pale brown solid (700 mg, 53% yield). LCMS (Method B): m/z=220.0 (M+H)⁺ (tRet: 1.15 min).

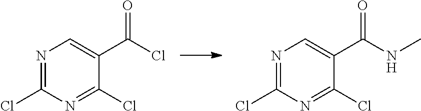

### 2,4-dichloro-N-methylpyrimidine-5-carboxamide

To a stirred solution of trimethylamine (20 mL, 146.397 mmol, 3.0 eq.) and methylamine (48.7 mL, 48.799 mmol, 1.0 eq.) in THE (20 mL) at −78° C. is added a solution of 2,4-dichloropyrimidine-5-carbonyl chloride (10 g, 48.799 mmol, 1.0 eq) in anhydrous THF (100 mL). The reaction mixture is stirred for 1 h at −78° C. The reaction progress is monitored by TLC. After completion of the reaction, the reaction mixture is quenched with water and extracted with EtOAc (200 mL). Then the organic layer is dried over Na₂SO₄ and concentrated under reduced pressure to afford crude compound. The crude compound is purified by MPLC using MeOH/CH₂Cl₂ (gradient 2-3% MeOH in CH₂Cl₂) to afford the desired compound as an off-white solid (2.50 g, 25% yield). LCMS (Method A): m/z=206.0 (M+H)⁺.

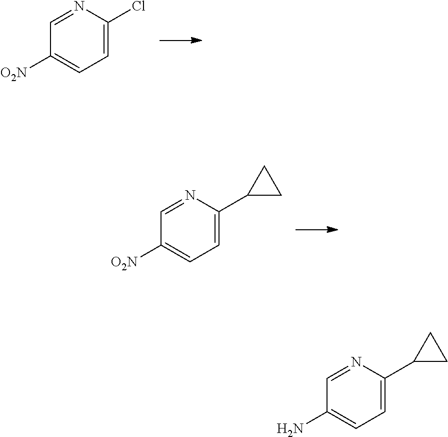 [SMILES: O=[N+]([O-])c1ccc(Cl)nc1 ; O=[N+]([O-])c1ccc(C2CC2)nc1 ; Nc1ccc(C2CC2)nc1]

### 2-cyclopropyl-5-nitropyridine

To a stirred solution of 2-chloro-5-nitropyridine (10.0 g, 63.09 mmol, 1.0 eq) in toluene (100 mL) are added cyclopropyl boronic acid (7.15 g, 83.28 mmol, 1.2 eq), tricyclohexyl phosphine (1.76 g, 6.309 mmol, 0.1 eq) and 2 M potassium triphosphate (49.3 g, 233.34 mmol, 3.70 eq) in water (116.0 mL) at RT. The reaction mixture is degassed for 15 min, finally adding palladium (II) acetate (1.14 g, 6.309 mmol, 0.1 eq) and again degassed for 2 min. Then the reaction mixture is stirred at 100° C. for 16 h. The reaction mixture turns into a black color solution. Completion of the reaction is monitored by TLC. The reaction mixture is quenched with water (100 mL) and ethyl acetate (300.0 mL), stirred for 5 min, then passed through celite pad, extracted and separated. Organic layer is dried over sodium sulfate and concentrated to afford the crude compound. The crude compound is purified by flash chromatography using a silica gel column with 8% ethyl acetate in hexane as an eluent to afford the desired compound as an off-white solid. LC-MS (Method-A): (M+H)⁺ 165.1, tRet: 2.38 min. Yield: 6.8 g; 65.0%.

### 6-cyclopropylpyridin-3-amine

To a stirred solution of 2-cyclopropyl-5-nitropyridine (5.8 g, 35.98 mmol, 1.0 eq) in methanol:THF (160 mL, 1:1) is added a solution of ammonium chloride (19.24 g, 359.8 mmol, 10.0 eq) in water (80.0 mL) and finally zinc is added (11.76 g, 179.94 mmol, 5.0 eq) portion wise for 30 min at RT. The reaction mixture is stirred at RT for 6 h. Reaction mixture turns into a light green color solution. Completion of the reaction is monitored by TLC. Excess of THF and methanol are distilled. Ethyl acetate (300.0 mL) and water (200) are added and the mixture is stirred for 5 min, then filtered through a celite bed, and washed with ethyl acetate (150.0 mL). Both the layers are extracted, organic layer is dried over anhydrous sodium sulfate, and concentrated to afford the crude compound. The crude compound is an amber color liquid. Yield: 4.0 g; 83.0%. LC-MS (Method C): (M+H)⁺ 135.1, tRet: 0.371 min.

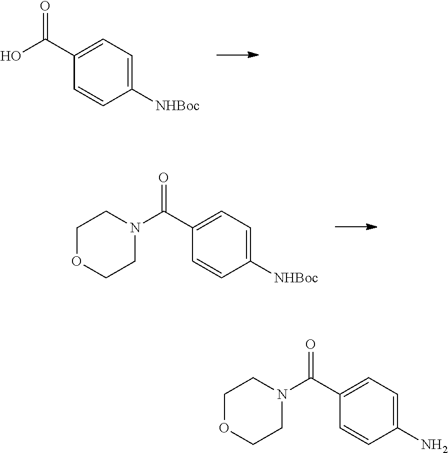 [SMILES: CC(C)(C)OC(=O)Nc1ccc(C(=O)O)cc1 ; CC(C)(C)OC(=O)Nc1ccc(C(=O)N2CCOCC2)cc1 ; Nc1ccc(C(=O)N2CCOCC2)cc1]

### Tert-Butyl (4-(morpholine-4-carbonyl)phenyl)carbamate

To a stirred solution of 4-((tert-butoxy carbonyl) amino) benzoic acid (5.0 g, 21.07 mmol, 1.0 eq.) in DMF (40 mL) are added DIPEA (8.95 mL, 52.68 mmol, 2.5 eq.), HATU (9.61 g, 25.28 mmol, 1.2 eq.) and morpholine (2.72 mL, 31.61 mmol, 1.5 eq.) at rt. The reaction mixture is stirred for 16 h at room temperature. The progress of the reaction is monitored by TLC. After completion of the reaction, water (100 ml) is added to the reaction mixture. The mixture is extracted with ethyl acetate (300 ml). The organic layer is washed with water (3×50 ml) and brine (30 ml) and then dried over Na₂SO₄. The organic layer is concentrated and dried completely to obtain an off white semi solid. This material is taken in water (100 mL) and stirred at rt for 30 min. The obtained solid is filtered and dried completely to afford the desired compound (6.0 g, 93%) as an off white solid. LC-MS (Method B): M+1: 307.1, tRet: 2.28 min.

### (4-aminophenyl)(morpholino) methanone

To a stirred solution of tert-butyl (4-(morpholine-4-carbonyl) phenyl) carbamate (1.0 g, 3.26 mmol, 1.0 eq.) in MeOH (10 mL) is added acetyl chloride (1.63 mL, 22.84 mmol, 7.0 eq.) dropwise at 0° C. The reaction mixture is stirred at rt for 6 h. Solid formation is observed during the reaction progress. The progress of the reaction is monitored by TLC. After completion of the reaction, the reaction mixture is concentrated under reduced pressure. The residue is triturated with diethyl ether (10 mL) and decanted. The obtained solid is dried completely to afford the desired compound (780 mg, 98%) as an off-white solid. LC-MS (Method B): M+1: 207.10, tRet: 0.68 min, tRet: 0.89 min.

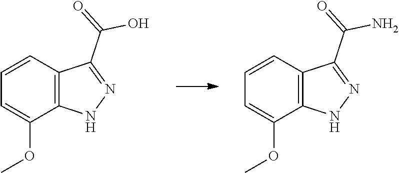

### 7-methoxy-1H-indazole-3-carboxamide

To a stirred solution of 7-methoxy-1H-indazole-3-carboxylic acid (0.2 g, 1.041 mmol, 1.0 eq.) in 5.0 mL of DMF are added ammonium chloride (0.281 g, 5.205 mmol, 5.0 eq.), HATU (0.593 g, 1.561 mmol, 1.5 eq.) and DIPEA (1.3 mL, 7.287 mmol, 7.0 eq.) at 0° C. The mixture is stirred at RT for 16 h. The progress of the reaction is monitored by TLC. The reaction mixture is quenched with 20 mL of water and extracted with (3×30 mL) of EtOAc. The combined organic layers are washed with brine solution (3×30 mL), dried over sodium sulphate and evaporated to dryness to afford the crude compound. The crude compound is taken in 10 mL of water, stirred for 30 min, filtered and dried well to afford a pale yellow solid. Yield: 0.150 g (75%); LC-MS (Method B): [M+H]⁺=192.00; tRet=1.18 min.

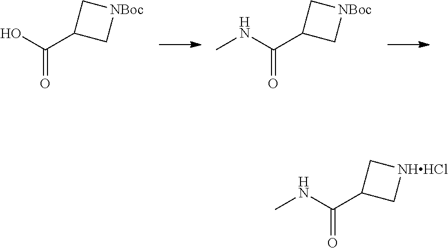

### Tert-Butyl 3-(methylcarbamoyl)azetidine-1-carboxylate

To a stirred solution of 1-(tert-butoxycarbonyl)azetidine-3-carboxylic acid (0.5 g, 2.486 mmol, 1.0 eq) in DMF (10 mL) is added EDC·HCl (0.71 g, 3.729 mmol, 1.5 eq), HOBT (0.50 g, 3.729 mmol, 1.5 eq) and DIPEA (2.16 mL, 12.430 mmol, 5.0 eq) followed by methylamine hydrochloride (0.25 g, 3.729 mmol, 1.5 eq) at RT. The reaction mixture turns into a pale yellow solution. The reaction mixture is stirred for 16 h at RT and the progress of the reaction is monitored by TLC. After completion of the reaction, the reaction mixture is diluted with ice cold water (30 mL) and extracted with EtOAc (2×50 mL), dried over anhydrous sodium sulphate and the organic solvent is evaporated to afford the desired compound as a white solid (0.26 g, impure). LCMS (Method-A): m/z=159.1 (M−56)⁺ tRet: 2.00 min.

### N-methylazetidine-3-carboxamide Hydrochloride

To a stirred solution of tert-butyl 3-(methylcarbamoyl)azetidine-1-carboxylate (0.26 g, 1.214 mmol, 1.0 eq) in DCM (10 mL) is added 4N HCl in dioxane (2.0 mL) at RT. Solid precipitates out. The reaction mixture is stirred for 2 h at RT and the progress of the reaction is monitored by TLC. After completion of the reaction, the reaction mass is concentrated under reduced pressure to furnish the crude compound (0.21 g). LCMS (Method B): m/z=115.1 (M−HCl)⁺ tRet: 0.25 min.

### General Procedure: Reaction Work-Up

General Reaction Work-ups: After completion of a reaction, volatile solvents may removed under reduced pressure to afford crude compound, which may be purified by chromatography (e.g., flash chromatography using, for instance, hexanes/ethyl acetate or ethyl acetate). Crude compound may be diluted with water, optionally ice cold, precipitate may be filtered, washed with a solvent (e.g., pentane), and dried under vacuum to afford desired compound. Alternatively, after completion of a reaction, the reaction mixture may be poured into water or diluted with water and the product precipitates. The solid formed may be filtered, optionally washed with a solvent (e.g., pentane), and dried under vacuum to afford the desired compound. Alternatively, after completion of a reaction, the reaction mixture may be partitioned between an organic solvent (e.g., ethyl acetate) and water, the organic layer(s) may be washed, dried, and concentrated to afford the crude compound, which may be purified by chromatography (e.g., flash chromatography using, for instance, hexanes/ethyl acetate or ethyl acetate). Compounds may also be purified or further purified by preparative HPLC and optionally dried by lyophilization for about 24 h to about 48 h. Compounds may also be purified by trituration (e.g., with DCM, MeOH, and n-hexane).

### General Procedure: Heteroaryl Amination Reaction

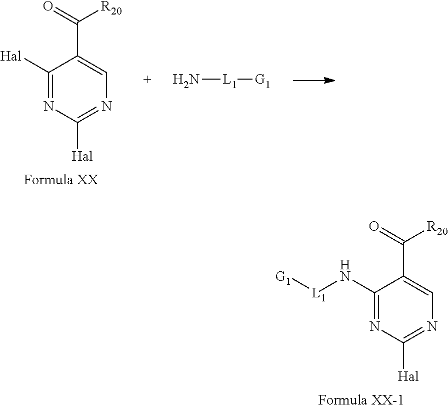

In a heteroaryl amination reaction, Hal is a halogen (e.g., Cl), R₂₀ is —OCH₂CH₃ or NHR₁, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, and R₂ is a 5- to 6-membered saturated heterocyclyl.

General Heteroaryl Amination Reaction: To a solution of Formula XX (1 eq) in an organic solvent (e.g., EtOH, THF, or dioxane) is added an amine base (e.g., triethylamine) (5 eq) and H₂N-L₁-G₁ (1-1.2 eq). The reaction mixture is stirred at room temperature or heated (with dioxane) for about 2.5 h to about 16 h. Reaction progress may be monitored by TLC and then work-up after completion.

General Heteroaryl Amination Reaction Alternative: To a solution of Formula XX (1 eq) in an organic solvent (e.g., methanol or n-BuOH) either cooled or at room temperature is added H₂N-L₁-G₁ (1-1.5 eq). The reaction mixture is stirred about 2 h to about 4 h at 80° C. Reaction progress may be monitored by TLC and then work-up after completion.

General Heteroaryl Amination Reaction Alternative: To a solution of Formula XX (1 eq) in an organic solvent (e.g., THF or N,N-dimethylacetamide) is added H₂N-L₁-G₁ (1.0-1.5 eq) and an amine base (e.g., DIPEA) (1.2-5 eq). The reaction mixture is stirred about 1 h at 120° C. in microwave irradiation. Alternatively, the reaction mixture is heated (e.g., to about 90° C. for about 4 h to about 6 h). Reaction progress may be monitored by TLC and then work-up after completion.

### Representative Heteroaryl Amination Reactions

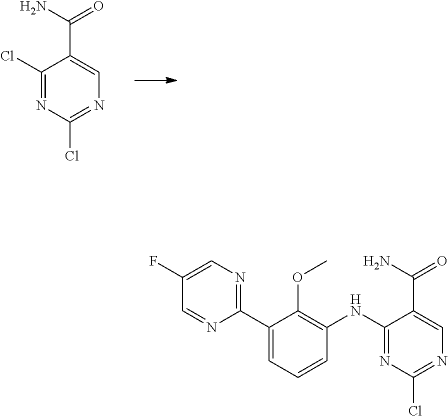 [SMILES: NC(=O)c1cnc(Cl)nc1Cl ; COc1c(Nc2nc(Cl)ncc2C(N)=O)cccc1-c1ncc(F)cn1]

### 2-chloro-4-((3-(5-fluoropyrimidin-2-yl)-2-methoxyphenyl)amino)pyrimidine-5-carboxamide (Intermediate 1)

To a stirred solution of 3-(5-fluoropyrimidin-2-yl)-2-methoxyaniline (0.38 g, 1.73 mmol, 1.0 eq) in ethanol is added triethylamine (1.20 mL, 8.65 mmol, 5.0 eq), then finally added 2,4-dichloropyrimidine-5-carboxamide (0.43 g, 2.25 mmol, 1.3 eq). The reaction mixture is stirred at RT for 5 h and stirred at 70° C. for 2 h. The reaction mixture turns into a light amber color solution. Completion of the reaction is monitored by TLC. The excess solvents are evaporated to minimal amount, then water (40.0 mL) is added and the mixture is stirred for 15 min. Off-white solid is formed, which is filtered and dried to afford the crude compound. The crude compound is washed with n-pentane (20.0 mL) to afford the desired compound. Yield: 0.21 g, 32% as an off-white solid. LC-MS: (M+H)⁺ (Method B): 374.95, tRet: 1.34, (M−H): 373.00.

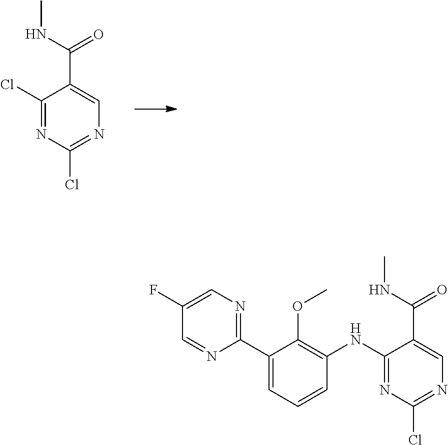 [SMILES: CNC(=O)c1cnc(Cl)nc1Cl ; CNC(=O)c1cnc(Cl)nc1Nc1cccc(-c2ncc(F)cn2)c1OC]

### 2-chloro-4-((3-(5-fluoropyrimidin-2-yl)-2-methoxyphenyl)amino)-N-methylpyrimidine-5-carboxamide (Intermediate 2)

To a stirred solution of 2,4-dichloro-N-methylpyrimidine-5-carboxamide (0.50 g, 2.44 mmol, 1.0 eq) in dioxane (20 mL) at RT are added triethylamine (0.68 mL, 4.88 mmol, 2.0 eq) and 3-(5-fluoropyrimidin-2-yl)-2-methoxyaniline (0.59 g, 2.68 mmol, 1.1 eq). Then the reaction mixture is heated at 100° C. for 16 h. Completion of the reaction is monitored by TLC. After completion of the reaction, the reaction mixture is concentrated under reduced pressure to afford the crude compound. The crude compound is purified by MPLC using DCM/MeOH (gradient 2-3% MeOH) to afford the desired compound as a pale yellow solid (0.50 g, 53%, yield). LCMS (Method B): m/z=388.95 (M+H), 387.00 (M−H) tRet: 1.38.

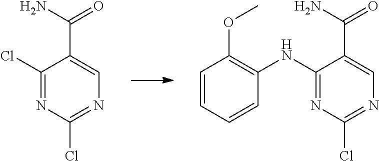 [SMILES: NC(=O)c1cnc(Cl)nc1Cl ; COc1ccccc1Nc1nc(Cl)ncc1C(N)=O]

### 2-chloro-4-((2-methoxyphenyl)amino)pyrimidine-5-carboxamide (Intermediate 3)

To a stirred solution of 2,4-dichloropyrimidine-5-carboxamide (1.0 g, 5.20 mmol, 1.0 eq) in THE (4 mL) is added 2-methoxyaniline (588.49 μL, 5.20 mmol, 1.0 eq) at RT. The reaction mixture is stirred at RT for 2.5 h. The progress of the reaction is monitored by TLC. The reaction mixture is diluted with cold water (100 mL), the obtained solid is filtered and dried completely to afford the desired compound as an off-white solid (850 mg, 59% yield). LCMS (Method A): m/z=279.1 (M+H)⁺, tRet: 2.37 min, HPLC (Method B): tRet: 6.84 min.

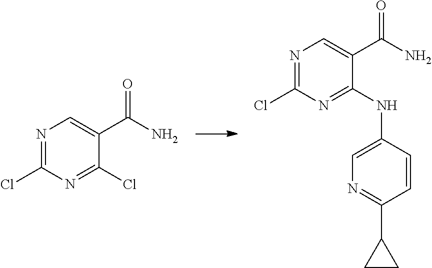 [SMILES: NC(=O)c1cnc(Cl)nc1Cl ; NC(=O)c1cnc(Cl)nc1Nc1ccc(C2CC2)nc1]

### 2-chloro-4-((6-cyclopropylpyridin-3-yl)amino)pyrimidine-5-carboxamide (Intermediate 4)

2,4-dichloropyrimidine-5-carboxamide (1.0 g, 5.20 mmol, 1.0 eq) and 6-cyclopropylpyridin-3-amine (0.908 g, 6.77 mmol, 1.3 eq) in methanol is stirred at 80° C. for 2 h. The reaction mixture turns into a light amber color solution. Completion of the reaction is monitored by TLC. The solvents are evaporated to dryness, then water (80.0 mL) is added and the mixture is stirred for 5 min. The solid formed is filtered and dried to afford crude compound. The crude compound is washed with n-pentane (20.0 mL), decanted and dried to afford the desired compound as an off-white solid. LC-MS (Method-D): (M−1): 288.0, tRet: 1.18. Yield: 1.0 g, 67%.

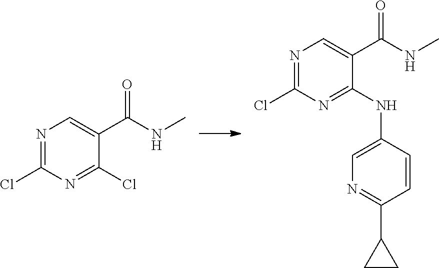 [SMILES: CNC(=O)c1cnc(Cl)nc1Cl ; CNC(=O)c1cnc(Cl)nc1Nc1ccc(C2CC2)nc1]

### 2-chloro-4-((6-cyclopropylpyridin-3-yl)amino)-N-methylpyrimidine-5-carboxamide (Intermediate 5)

To a stirred solution of 2,4-dichloro-N-methylpyrimidine-5-carboxamide (0.300 g, 1.463 mmol, 1.0 eq.) in MeOH (10 mL), add 6-cyclopropylpyridin-3-amine (0.294 g, 2.194 mmol, 1.5 eq.). The reaction mixture is stirred for 2 h at 80° C. and the reaction progress is monitored by TLC. Once the starting material disappears, the reaction mixture is evaporated to afford the crude product. The crude product is purified by flash chromatography using 40% EtOAc in hexane as an eluent to afford the desired compound (0.300 g, 68%) as a white solid. LC-MS (Method B): M+1: 304.00.

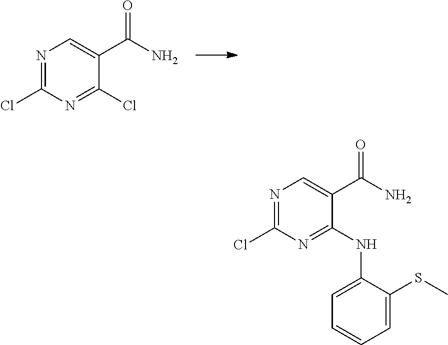 [SMILES: NC(=O)c1cnc(Cl)nc1Cl ; CSc1ccccc1Nc1nc(Cl)ncc1C(N)=O]

### 2-chloro-4-((2-(methylthio)phenyl)amino)pyrimidine-5-carboxamide (Intermediate 6)

To a stirred solution of 2,4-dichloropyrimidine-5-carboxamide (0.5 g, 2.618 mmol, 1.0 eq) in THF (10 mL) is added DIPEA (2.28 mL, 13.09 mmol, 5.0 eq) followed by 2-(methylthio)aniline (0.54 g, 3.927 mmol, 1.5 eq). Then the reaction mixture turns into a brown suspension and the vessel is irradiated in microwave instrument for 1 h at 120° C. The progress of the reaction is monitored by TLC. After completion of the reaction, the reaction mixture is quenched with 30 mL of water and extracted with (2×50 mL) of EtOAC. The combined organic layers are dried over sodium sulphate and concentrated under reduced pressure to afford the desired compound as an off-white solid (0.55 g, 71% yield). LCMS (Method-A): m/z=295.00 (M+H)⁺ tRet: 2.38 min.

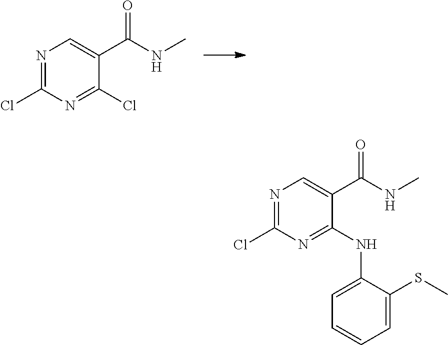 [SMILES: CNC(=O)c1cnc(Cl)nc1Cl ; CNC(=O)c1cnc(Cl)nc1Nc1ccccc1SC]

### 2-chloro-N-methyl-4-((2-(methylthio)phenyl)amino)pyrimidine-5-carboxamide (Intermediate 7)

To a stirred solution of 2,4-dichloro-N-methylpyrimidine-5-carboxamide (0.750 g, 3.658 mmol, 1.0 eq) in THE (10 ml), 2-(methylthio)aniline (0.610 g, 4.389 mmol, 1.2 eq) and DIPEA (0.956 ml, 5.487 mmol, 1.5 eq) are added. Then the reaction mixture is stirred for 1 h at 120° C. in microwave irradiation. The reaction mixture turns into a clear yellow solution. Completion of the reaction is monitored by TLC. After completion of the reaction, the volatile solvents are evaporated under reduced pressure to afford the crude compound. The crude compound is purified by MPLC using EtOAc/hexane (gradient 30-40% EtOAc) to afford the desired compound as a yellow solid (900 mg, 80% yield). LCMS (Method B): m/z=309.00 (M+H)⁺.

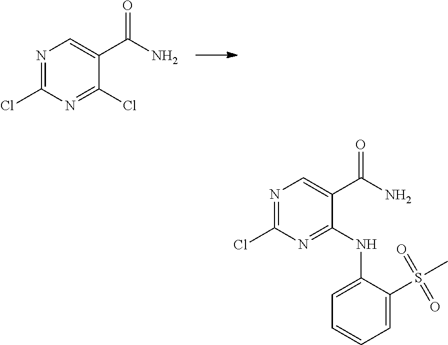 [SMILES: NC(=O)c1cnc(Cl)nc1Cl ; CS(=O)(=O)c1ccccc1Nc1nc(Cl)ncc1C(N)=O]

### 2-chloro-4-((2-(methylsulfonyl)phenyl)amino)pyrimidine-5-carboxamide (Intermediate 8)

To a stirred solution of 2,4-dichloropyrimidine-5-carboxamide (0.50 g, 2.62 mmole, 1.0 eq) in N,N′-dimethylacetamide (15 mL), 2-(methylsulfonyl)aniline (0.45 g, 2.62 mmol, 1.0 eq) and DIPEA (2.30 mL, 13.09 mmol, 5.0 eq) are added. The reaction mixture is stirred for 6 h at 90° C. The reaction mixture turns to clear brown solution. The progress of the reaction is monitored by TLC. After completion of the reaction, the reaction mixture is quenched with cold water (50 mL) and extracted into ethyl acetate (30 mL×3). The ethyl acetate layer is washed with cold water (50 mL) and brine solution (50 mL). Organic layer is dried over anhydrous sodium sulphate and concentrated to afford the crude compound. The crude compound is purified by MPLC using hexane/EtOAc (gradient 40-50% EtOAc) to afford the desired compound as a white solid (0.17 g, impure). LCMS (Method A): m/z=327.1 (M+H)⁺ tRet: 2.11.

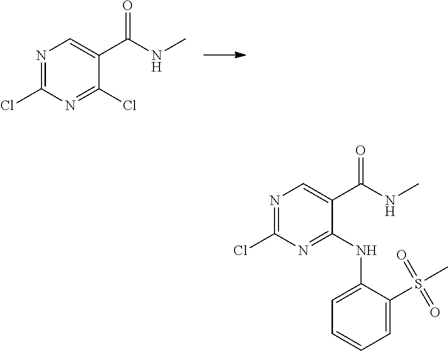 [SMILES: CNC(=O)c1cnc(Cl)nc1Cl ; CNC(=O)c1cnc(Cl)nc1Nc1ccccc1S(C)(=O)=O]

### 2-chloro-N-methyl-4-((2-(methylsulfonyl)phenyl)amino)pyrimidine-5-carboxamide (Intermediate 9)

To a stirred solution of 2,4-dichloro-N-methylpyrimidine-5-carboxamide (0.500 g, 2.439 mmol, 1.0 eq.) in N,N′-dimethylacetamide (4 ml) is added 2-(methylsulfonyl)aniline (0.417 g, 2.439 mmol, 1.0 eq.) and DIPEA (2.124 ml, 12.195 mmol, 5.0 eq.). Reaction mixture is stirred for 4 h at 90° C. Reaction mixture turns to clear brown solution. Reaction progress is monitored by TLC. After completion of the reaction, the reaction mixture is quenched with cold water solution, then extracted into ethyl acetate (100 ml×3). Ethyl acetate layer is washed with cold water and brine solution. Organic layer is dried over sodium sulphate and concentrated to afford the crude material. The crude material is purified by flash chromatography using 60-70% EtOAc in hexane as an eluent to afford the desired compound (0.180 g, impure) as a yellow solid. LC-MS (Method B): [M+H]⁺: 341.1, [M+3H]343.0, [M−H]⁺: 339.1, [M−H+3]+: 341.0, tRet: 2.214 min.

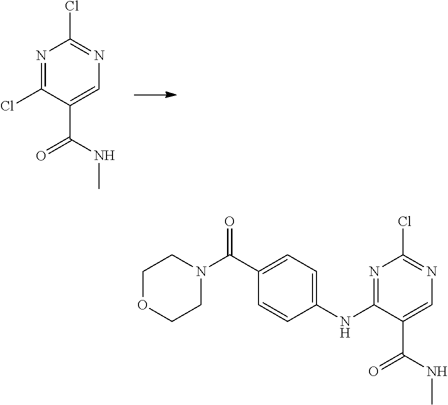 [SMILES: CNC(=O)c1cnc(Cl)nc1Cl ; CNC(=O)c1cnc(Cl)nc1Nc1ccc(C(=O)N2CCOCC2)cc1]

### 2-chloro-N-methyl-4-((4-(morpholine-4-carbonyl)phenyl)amino)pyrimidine-5-carboxamide (Intermediate 10)

To a stirred solution of 2,4-dichloro-N-methylpyrimidine-5-carboxamide (0.250 g, 1.219 mmole, 1.0 eq.) in ACN (5 ml) is added (4-aminophenyl)(morpholino)methanone hydrochloride (0.354 g, 1.462 mmol, 1.2 eq.) and DIPEA (0.637 ml, 3.657 mmol, 3.0 eq.). The reaction mixture is stirred for 16 h at 80° C. Reaction mixture turns to a clear yellow solution. Reaction is monitored by TLC. After completion of the reaction, the reaction mixture is quenched with cold water solution. Then extracted into ethyl acetate (50 ml×3). Ethyl acetate layer is washed with cold water and brine solution. Organic layer is dried over sodium sulphate and concentrated to afford crude material. The crude material is purified by flash chromatography using 70% EtOAc in hexane to afford the desired compound (0.250 g 55%) as a white solid. LC-MS (Method B): M+1: 376.00, tRet: 1.240 min.

### General Procedure: Coupling Reaction

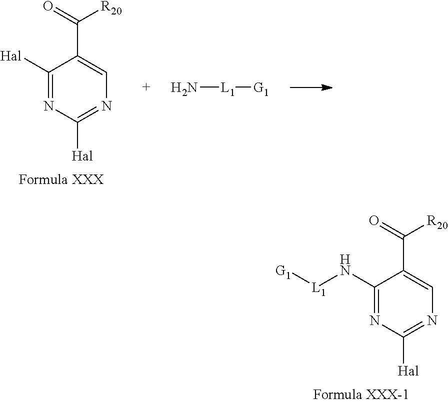

In a coupling reaction, Hal is a halogen (e.g., Cl), R₂₀ is —OCH₂CH₃ or NHR₁, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, and R₂ is a 5- to 6-membered saturated heterocyclyl.

General Coupling Reaction: To a solution of Formula XXX (1 eq), H₂N-L₁-G₁ (1 eq), and a base (e.g., cesium carbonate) (1.5 eq) in an organic solvent (e.g., dioxane) is added a palladium catalyst (e.g., Pd₂(dba)₃) (0.05 eq) and an organophosphorus compound (e.g., xantphos) (0.1 eq). The reaction mixture is heated (e.g., 110° C.) for about 1 h to about 2 h. Reaction progress may be monitored by TLC and then work-up after completion.

### Representative Coupling Reaction

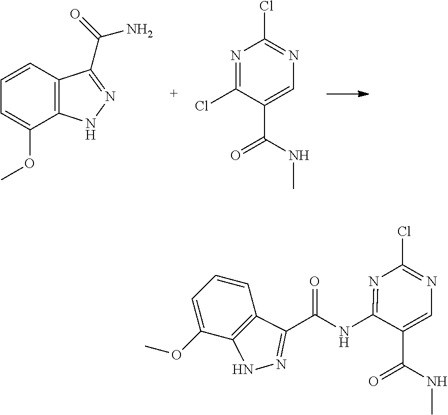 [SMILES: COc1cccc2c(C(N)=O)n[nH]c12 ; CNC(=O)c1cnc(Cl)nc1Cl ; CNC(=O)c1cnc(Cl)nc1NC(=O)c1n[nH]c2c(OC)cccc12]

### N-(2-chloro-5-(methylcarbamoyl)pyrimidin-4-yl)-7-methoxy-1H-indazole-3-carboxamide (Intermediate 11)

A stirred solution of 2,4-dichloro-N-methylpyrimidine-5-carboxamide (0.4 g, 1.951 mmol, 1.0 eq.), 7-methoxy-1H-indazole-3-carboxamide (0.372 g, 1.951 mmol, 1.0 eq.) and cesium carbonate (0.953 g, 0.364 mmol, 1.5 eq.) in 15 mL of dry 1,4-dioxane is degassed with nitrogen gas for 5 min and then Pd₂(dba)₃ (89 mg, 0.0975 mmol, 0.05 eq.) and Xantphos (112 mg, 0.195 mmol, 0.1 eq.) are added. The mixture is heated to reflux at 110° C. for 1 h. The color of the reaction mixture changes from red wine color to pale yellow. The progress of the reaction is monitored by TLC. The solid formed is filtered and washed with 30 mL of 1,4-dioxane and the filtrate is concentrated to afford the crude compound. The crude compound is purified by flash chromatography using a silica gel column and eluting with 2% methanol in DCM to afford the desired compound as an off-white solid. Yield: 0.1 g (impure). LC-MS (Method A): M+1=361, tRet=2.785 min; HPLC (Method B): tRet=6.570 min.

### General Procedure: Sulfur Oxidation

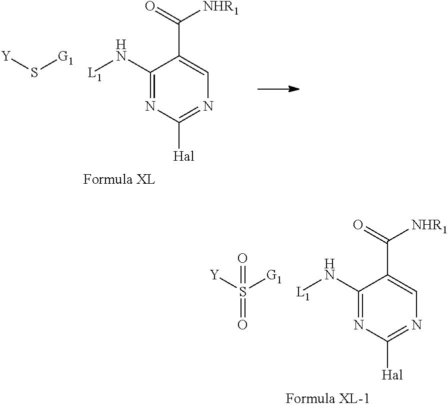

In a sulfur oxidation, Hal is a halogen (e.g., Cl), R₁ is H or C₁₋₃-alkyl, L₁ is a bond, G₁ is an aryl or a 6- to 9-membered heteroaryl substituted with —SC₁₋₃-alkyl, and Y is C₁₋₃-alkyl.

General Sulfur Oxidation: To a solution of Formula XL (1 eq) in an organic solvent (e.g., EtOAc) at 0° C. or at room temperature is added an oxidizing agent (e.g., mCPBA) (1.2-5 eq.). The reaction mixture is stirred about 2 hours to about 16 hours at room temperature. Reaction progress may be monitored by TLC and then work-up after completion.

### Representative Syntheses of Sulfur Oxidation

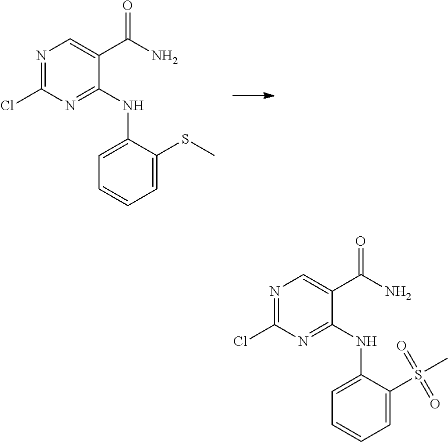 [SMILES: CSc1ccccc1Nc1nc(Cl)ncc1C(N)=O ; CS(=O)(=O)c1ccccc1Nc1nc(Cl)ncc1C(N)=O]

### 2-chloro-4-((2-(methylsulfonyl)phenyl)amino)pyrimidine-5-carboxamide (Intermediate 12)

To a stirred solution of 2-chloro-4-((2-(methylthio)phenyl)amino)pyrimidine-5-carboxamide (0.55 g, 1.870 mmol, 1.0 eq) in EtOAc (20 mL) is added mCPBA (1.61 g, 9.352, 5.0 eq). Then the reaction mixture turns into a white suspension and is stirred for 16 h at RT. The progress of the reaction is monitored by TLC. After completion of the reaction, the reaction is diluted with EtOAc (30 mL) and washed with sat. Na₂S₂O₃/NaHCO₃ (1:1 ratio, 30 mL), sat. NaHCO₃ (30 mL) and brine (30 mL) sequentially. The organic layer is dried over anhydrous Na₂SO₄, filtered and evaporated to dryness. The solid is diluted with DCM/hexane (1:5 ratio, 10 mL), filtered and dried under high vacuum to afford the desired compound as an off-white solid (0.45 g, 74% yield). LCMS (Method-B): m/z=326.90 (M+H)⁺, tRet: 1.26 min.

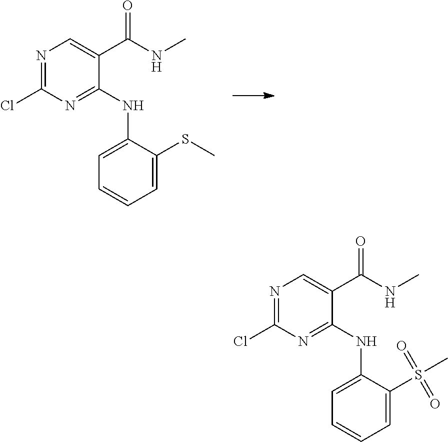 [SMILES: CNC(=O)c1cnc(Cl)nc1Nc1ccccc1SC ; CNC(=O)c1cnc(Cl)nc1Nc1ccccc1S(C)(=O)=O]

### 2-chloro-N-methyl-4-((2-(methylsulfonyl)phenyl)amino)pyrimidine-5-carboxamide (Intermediate 13)

To a stirred solution of 2-chloro-N-methyl-4-((2-(methylthio)phenyl)amino)pyrimidine-5-carboxamide (0.500 g, 1.623 mmol, 1.0 eq) in EtOAc (40 ml), m-chloroperbenzoic acid (0.647 g, 4.869 mmol, 3.0 eq) is added at 0° C. The reaction mixture is stirred for 2 h at rt. The reaction mixture turns to white precipitate. Completion of the reaction is monitored by TLC. After completion of the reaction, EtOAc (100 ml) is added to the reaction mixture, then the organic layer is washed with aq. sat. NaHCO₃ solution (2×50 ml) and brine solution (50 ml), dried over Na₂SO₄ and evaporated to afford crude material. The crude material is purified by MPLC using EtOAc/hexane (gradient 50-60% EtOAc) to afford the desired compound as an off white solid (350 mg, 63% yield). LCMS (Method B): m/z=340.90 (M+H)⁺.

### General Procedure: Second Heteroaryl Amination Reaction

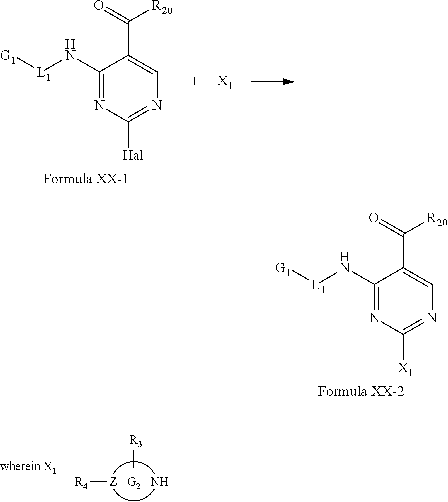

In a second heteroaryl amination reaction, Hal is a halogen (e.g., Cl), R₂₀ is —OCH₂CH₃ or NHR₁, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, R₂ is a 5- to 6-membered saturated heterocyclyl, G₂ is a 4- to 12-membered saturated heterocyclyl, Z is CH or N, R₃ is H or C₁₋₃-alkyl, R₄ is H or —C(O)R₅, and R₅ is C₁₋₃-alkyl, OH, NH₂, —NHC₁₋₃-alkyl, C₁₋₆-alkoxy, or C₃-6-cycloalkyl.

Second General Heteroaryl Amination Reaction: To a solution of Formula XX-1 (1 eq) in an organic solvent (e.g., n-butanol or N,N-dimethyacetamide) is added a base (e.g., DIPEA or K₂CO₃) (3-15 eq) and X₁ (1.5-5 eq.). The reaction mixture is stirred and heated (e.g., from about 100° C. to about 150° C.) for about 1.5 to about 15 hours. Heating may be done with microwave irradiation, which may shorten the reaction time (e.g., to about 1.5 hours). Reaction progress may be monitored by TLC and then work-up after completion.

### General Procedure: Saponification

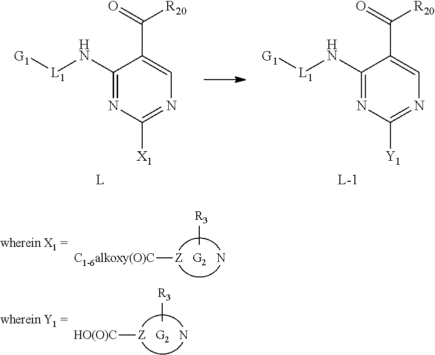

In a saponification reaction, R₂₀ is —OCH₂CH₃ or NHR₁, R₁ is H or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, R₂ is a 5- to 6-membered saturated heterocyclyl, G₂ is a 4- to 12-membered saturated heterocyclyl, Z is CH or N, G₂ is substituted with —C(O)C₁₋₆-alkoxy, and R₃ is H or C₁₋₃-alkyl.

Saponification Reaction: To a solution of Formula L (1 eq) in an organic solvent or organic solvent mixture (e.g., THF and water and optionally MeOH) is added hydroxide (e.g., LiOH·H₂O) (3-10 eq.). The reaction mixture is stirred at room temperature or heated to 30-50° C. for about 4 hours to about 16 hours. Reaction progress may be monitored by TLC and then work-up after completion.

### General Procedure: N-t-Boc Deprotection

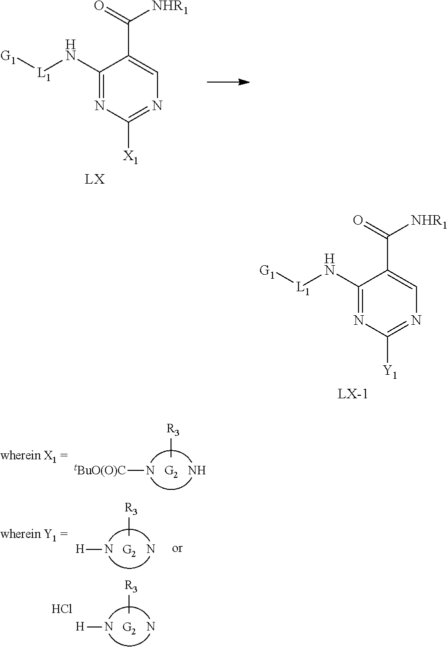

In an N-t-Boc deprotection, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens, R₂ is a 5- to 6-membered saturated heterocyclyl, G₂ is a 4- to 12-membered saturated heterocyclyl substituted with —C(O)O^{t}Bu, and R₃ is H or C₁₋₃-alkyl.

N-t-Boc Deprotection: N-t-Boc may be removed by an alcoholic solution (e.g., MeOH) of an acyl chloride (e.g., acetyl chloride). Acetyl chloride (7-30 eq) is added slowly to MeOH at 0° C. or room temperature and stirred at 0° C. or room temperature for 30 minutes. Formula LX (1 eq) is added slowly to the acetyl chloride/MeOH mixture at 0° C. or room temperature. The reaction may be stirred at 0° C. for 30 minutes. The reaction is then stirred at room temperature or under gentle heat (25-30° C.) for about 4 h to about 16 h. Reaction progress may be monitored by TLC and then work-up after completion.

N-t-Boc may also be removed by reaction with acid (e.g., HCl). To a stirred solution of Formula LX (1 eq) in an organic solvent (e.g., DCM) is added an acid (e.g., HCl in dioxane, e.g., 2M or 4N HCl in dioxane) (3-10 eq) at room temperature or at 0° C. The reaction mixture is stirred at room temperature or under gentle heat (25-30° C.) for about 2 hours to about 3 hours. Reaction progress may be monitored by TLC and then work-up after completion.

### General Procedure: Amide Formation Reaction

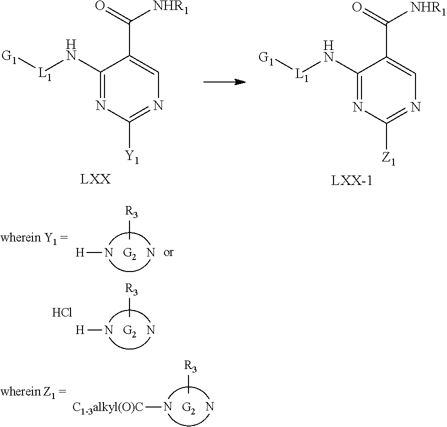

In an amide formation reaction, R₁ is H, OH, or C₁₋₃-alkyl, L₁ is a bond or carbonyl, G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, and 5- to 6-membered heteroaryl optionally substituted with one or more halogens, R₂ is a 5- to 6-membered saturated heterocyclyl, G₂ is a 4- to 12-membered saturated heterocyclyl, and R₃ is H or C₁₋₃-alkyl.

Amide Formation Reaction: To a stirred solution of Formula LXX (1 eq) in an organic solvent (e.g., DCM) at 0° C. or at room temperature is added a base (4-8 eq) (e.g., pyridine, triethylamine, or DIPEA). The mixture is stirred for about 5 minutes to about 20 minutes and then an acyl halide (1.3-2 eq) (e.g., acetyl chloride) or anhydride (10 eq) (e.g., acetic anhydride) is added at 0° C. or at room temperature. The reaction is stirred at RT or under heat (25-30° C. or 80° C. with acetic anhydride) for about 1 h to about 16 h. Reaction progress may be monitored by TLC and then work-up after completion.

### Example 1

### Example 1A

### (R)-4-((3-(5-fluoropyrimidin-2-yl)-2-methoxyphenyl)amino)-2-(3-(methylcarbamoyl)pyrrolidin-1-yl)pyrimidine-5-carboxamide (Table 1, Compound No. 53)

To a stirred solution of 2-chloro-4-((3-(5-fluoropyrimidin-2-yl)-2-methoxyphenyl)amino)pyrimidine-5-carboxamide (70 mg, 0.18 mmol, 1.0 eq) in n-butanol (5 mL) in a sealed tube are added DIPEA (158.75 μL, 0.93 mmol) and (R)—N-methylpyrrolidine-3-carboxamide (35.91 mg, 0.28 mmol, 1.5 eq) at RT. The reaction mixture is heated at 150° C. for 16 h. The progress of the reaction is monitored by TLC. The reaction mixture is concentrated. The residue is dissolved in ethyl acetate (50 mL), washed with water (10 mL) and brine (10 mL), dried over anhydrous Na₂SO₄, concentrated and dried to afford the crude compound. The crude compound is purified by flash chromatography. The product is eluted with 10% MeOH in DCM. The fractions containing the product are concentrated under reduced pressure to afford the crude compound. The crude compound is purified by preparative HPLC. The collected fractions are lyophilized for 48 h to afford the desired compound as an off-white solid (15 mg, 17% yield). ¹H NMR (400 MHz, DMSO-d₆): δ 12.06 (s, 1H), 9.02 (s, 2H), 8.87-8.81 (m, 1H), 8.64 (s, 1H), 8.03-7.92 (m, 2H), 7.33-7.21 (m, 3H), 3.81-3.73 (m, 2H), 3.69 (s, 3H), 3.66-3.49 (m, 2H), 3.08-3.01 (m, 1H), 2.62-2.60 (m, 3H), 2.17-2.05 (m, 2H). LCMS (Method B): m/z=467.15 (M+H)⁺, tRet: 1.14 min, HPLC (Method B): tRet: 5.38 min.

### Example 1B

### (S)-4-((3-(5-fluoropyrimidin-2-yl)-2-methoxyphenyl)amino)-2-(3-(methylcarbamoyl)pyrrolidin-1-yl)pyrimidine-5-carboxamide (Table 1, Compound No. 52)

To a stirred solution of 2-chloro-4-((3-(5-fluoropyrimidin-2-yl)-2-methoxyphenyl)amino)pyrimidine-5-carboxamide (70 mg, 0.18 mmol, 1.0 eq) in n-butanol (5 mL) in a sealed tube are added DIPEA (158.75 μL, 0.93 mmol) and (S)—N-methylpyrrolidine-3-carboxamide hydrochloride (35.91 mg, 0.28 mmol, 1.5 eq) at RT. The reaction mixture is heated at 150° C. for 16 h. The progress of the reaction is monitored by TLC. The reaction mixture is concentrated. The residue is dissolved in ethyl acetate (50 mL), washed with water (10 mL) and brine (10 mL), dried over anhydrous Na₂SO₄, concentrated and dried completely. The crude compound is purified by flash chromatography. The product is eluted with 10% MeOH in DCM. The fractions containing the product are concentrated under reduced pressure and dried to afford the crude compound The crude compound is purified by preparative HPLC. The collected fractions are lyophilized for 48 h to afford the desired compound as an off-white solid (145 mg, 18% yield). ¹H NMR (400 MHz, DMSO-d₆): δ 12.06 (s, 1H), 9.03 (s, 2H), 8.87-8.81 (m, 1H), 8.64 (s, 1H), 8.03-7.92 (m, 2H), 7.33-7.21 (m, 3H), 3.81-3.77 (m, 2H), 3.68 (s, 3H), 3.59-3.55 (m, 2H), 3.08-3.01 (m, 1H), 2.62-2.60 (m, 3H), 2.19-2.02 (m, 2H). LCMS (Method B): m/z=467.15 (M+H), tRet: 1.16 min, HPLC (Method B): tRet: 5.39 min.

### Example 2

### (R)-4-((3-(5-fluoropyrimidin-2-yl)-2-methoxyphenyl)amino)-N-methyl-2-(3-(methylcarbamoyl)pyrrolidin-1-yl)pyrimidine-5-carboxamide (Table 1, Compound No. 5)

To a stirred solution of 2-chloro-4-((3-(5-fluoropyrimidin-2-yl)-2-methoxyphenyl)amino)-N-methylpyrimidine-5-carboxamide (0.07 g, 0.180 mmol, 1.0 eq) in nBuOH (10 mL) is added (R)—N-methylpyrrolidine-3-carboxamide hydrochloride (0.044 g, 0.270 mmol, 1.5 eq) followed by DIPEA (0.47 mL, 2.700 mmol, 15 eq). Then the reaction mixture turns into a clear solution and is stirred for 16 h at 150° C. The progress of the reaction is monitored by TLC. The reaction mixture is cooled to RT and volatile solvents are removed under reduced pressure. The obtained mixture is diluted with water (20 mL), the aqueous layer is extracted with ethyl acetate (30 mL×3), organic phases are washed with brine (20 mL×1), dried over sodium sulphate and evaporated to dryness to afford the crude compound. The crude compound is purified by MPLC using DCM/MeOH (gradient 5-10% MeOH) to afford the desired compound as an off-white solid (0.05 g, 58% yield). ¹H NMR (400 MHz, DMSO-d₆): δ=11.85 (s, 1H), 9.03 (s, 2H), 8.84-8.79 (m, 1H), 8.57 (s, 1H), 8.36-8.35 (m, 1H), 8.02-7.98 (m, 1H), 7.32-7.30 (m, 1H), 7.24-7.20 (m, 1H), 3.82-3.76 (m, 1H), 3.70 (s, 3H), 3.69-3.41 (m, 3H), 3.08-3.01 (m, 1H), 2.76 (d, J=4.4 Hz, 3H), 2.67-2.61 (m, 3H), 2.18-2.05 (m, 2H). LCMS (Method-A): m/z=481.2 (M+H)⁺, tRet: 1.94 min. HPLC (Method-B): tRet: 5.40 min.

### Example 3

### (S)-4-((3-(5-fluoropyrimidin-2-yl)-2-methoxyphenyl)amino)-N-methyl-2-(3-(methylcarbamoyl)pyrrolidin-1-yl)pyrimidine-5-carboxamide (Table 1, Compound No. 7)

To a stirred solution of 2-chloro-4-((3-(5-fluoropyrimidin-2-yl)-2-methoxyphenyl)amino)-N-methylpyrimidine-5-carboxamide (90.0 mg, 0.231 mmol, 1.0 eq) in n-butanol (6.0 mL) in a sealed tube is added (S)—N-methylpyrrolidine-3-carboxamide hydrochloride (57.28 mg, 0.346 mmol, 1.5 eq) at RT, then finally added N,N-diisopropylethylamine (0.31 mL, 1.848 mmol, 8.0 eq). The reaction mixture is stirred and heated at 150° C. for 16 h. Color of the reaction turns into a light yellow color solution. Completion of the reaction is monitored by TLC. Excess solvents are evaporated to afford the crude compound. Residue is diluted with ethyl acetate (50.0 mL) and washed with water (50.0 mL×2) times, extracted and separated, organic layer is dried over anhydrous sodium sulfate and concentrated to afford the crude compound. The crude compound is purified by flash chromatography, eluting with 15% methanol in DCM. The crude is washed with acetonitrile (5.0 mL) and hexane (5.0 mL), decanted and dried to afford the desired compound. Yield: 50.0 mg (45%) (off-white solid).

¹H NMR (400 MHz, DMSOd₆) δ 11.85 (s, 1H), 8.85 (s, 2H), 8.83-8.79 (m, 1H), 8.58 (s, 1H), 8.36-8.35 (s, J=4 Hz, dH), 8.02-7.99 (m, 1H), 7.32-7.30 (m, 1H), 7.24-7.20 (m, 1H), 3.79-3.76 (m, 1H), 3.7 (s, 1H), 3.66-3.54 (m, 3H), 3.08-3.01 (m, 1H), 2.77-2.76 (d, J=4 Hz, 3H), 2.67-2.61 (m, 3H), 2.17-2.14 (m, 2H). LC-MS (Method B): tRet=5.412, HPLC (Method B): tRet=5.412.

### Example 4

### 4-((6-cyclopropylpyridin-3-yl)amino)-2-(pyrrolidin-1-yl)pyrimidine-5-carboxamide (Table 1, Compound No. 2)

Pale yellow clear solution of 2-chloro-4-((6-cyclopropylpyridin-3-yl) amino)pyrimidine-5-carboxamide (0.1 g, 0.523 mmol, 1.0 eq.) in 3.0 mL of pyrrolidine is heated to reflux at 100° C. in a sealed tube for 1 h. The progress of the reaction is monitored by TLC. Excess solvent is evaporated to the dryness and obtained solids are taken in 10 mL of water, stirred for 15 min, filtered, washed with n-pentane and dried well to afford the desired compound as an off-white solid. Yield: 0.1 g (59%). LC-MS (Method B): [M+H]=325.05; tRet=1.13 min; HPLC (Method B): tRet=5.085 min.

### Example 5

### (S)-4-((6-cyclopropylpyridin-3-yl)amino)-2-(3-(methylcarbamoyl)pyrrolidin-1-yl)pyrimidine-5-carboxamide (Table 1, Compound No. 3)

To a stirred solution of 2-chloro-4-((6-cyclopropylpyridin-3-yl)amino)pyrimidine-5-carboxamide (0.1 g, 0.345 mmol, 1.0 eq.) in 5.0 mL of 1-butanol is added (S)—N-methylpyrrolidine-3-carboxamide hydrochloride (0.105 g, 0.693 mmol, 1.8 eq.) and DIPEA (0.3 mL, 1.727 mmol, 5.0 eq.). The reaction mixture is stirred at 100° C. in a sealed tube to form a clear solution and stirred for 2 h. Off-white solids precipitate in the reaction mixture. The progress of the reaction is monitored by TLC. The excess of solvent is evaporated and the obtained solid is taken in 30 mL of water and stirred vigorously for 30 min. The solid is filtered, washed with 5 mL of diethyl ether and 5 mL of n-pentane and dried well under vacuum to afford the desired compound as an off-white solid. Yield: 0.09 g (68%). LC-MS(Method A): [M+H]⁺=382.1; tRet=1.680 min; HPLC (Method C): tRet=5.389 min.

### Example 6

4-((6-cyclopropylpyridin-3-yl)amino)-N-methyl-2-(3-(methylcarbamoyl)pyrrolidin-1-yl)pyrimidine-5-carboxamide (Table 1, Compound No. 1)

To a stirred solution of 2-chloro-4-((6-cyclopropylpyridin-3-yl)amino)-N-methylpyrimidine-5-carboxamide (0.200 g, 0.659 mmol, 1.0 eq.) in BuOH (8 mL), N-methylpyrrolidine-3-carboxamide (0.169 g, 1.318 mmol, 2.0 eq.) and K₂CO₃ (0.455 g, 3.295 mmol, 5.0 eq.) are added. Then the reaction mixture is stirred for 1.5 h at 150° C. in microwave irradiation. Completion of the reaction is monitored by TLC. Reaction volatile solvents are evaporated. The crude product is purified by flash chromatography using 60% EtOAc in hexane as an eluent to afford the desired compound (0.150 g, 56%) as a white solid. ¹H NMR (400 MHz, DMSO-d₆): δ ppm=11.44 (d, 1H, J=12.0 Hz), 8.70 (d, J=16.0 Hz, 1H), 8.56 (s, 1H), 8.40-8.39 (m, 1H), 8.10-7.96 (m, 2H), 7.23 (d, J=8.0 Hz, 1H), 3.78-3.69 (m, 2H), 3.54-3.48 (m, 2H), 3.05-2.99 (m, 1H), 2.76 (d, J=4.0 Hz, 3H), 2.62-2.58 (m, 3H), 2.13-2.00 (m, 3H), 0.92-0.83 (in, 4H) and LC-MS (Method B): M+1: 396.15, tRet: 1.151 min and HPLC (Method E): tRet: 5.256 min.

### Example 7

### (R)-4-((6-cyclopropylpyridin-3-yl)amino)-2-(3-(methylcarbamoyl)pyrrolidin-1-yl)pyrimidine-5-carboxamide (Table 1, Compound No. 4)

Synthesis is performed similar to Second Heteroaryl Amination Reaction and Examples above using Intermediate 4 (0.140 g, 0.484 mmol, 1.0 eq), (R)—N-methylpyrrolidine-3-carboxamide hydrochloride (1.5 eq), n-butanol (10 mL), and DIPEA (5 eq) and stirring at 140° C. for 2 h. Desired compound is an off white solid. Yield: 0.120 g, 65.04%. LC-MS (M+H)⁺: 382.10; tRet: 1.08 min. HPLC: tRet: 5.39 min.

### Example 8

### (R)-4-((6-cyclopropylpyridin-3-yl)amino)-N-methyl-2-(3-(methylcarbamoyl)pyrrolidin-1-yl)pyrimidine-5-carboxamide (Table 1, Compound No. 9)

Synthesis is performed similar to Second Heteroaryl Amination Reaction and Examples above using Intermediate 5 (0.120 g, 0.395 mmol, 1.0 eq), (R)—N-methylpyrrolidine-3-carboxamide hydrogen chloride (5.0 eq), BuOH (3 mL), and DIPEA (5 eq) and stirring for 1.5 h at 150° C. under microwave irradiation. Desired compound is a white solid (0.020 g, 54%). ¹H NMR (400 MHz, DMSO-d₆): δ ppm=11.44 (d, J=8.8 Hz, 1H), 8.70 (d, J=16.4 Hz, 1H), 8.56 (s, 1H), 8.40 (d, J=3.6, 1H), 8.09-7.97 (m, 2H), 7.23 (d, J=8.4 Hz, 1H), 3.75-3.67 (m, 2H), 3.56-3.45 (m, 2H), 3.05-2.99 (m, 1H), 2.76 (d, J=4.0 Hz, 3H), 2.60 (bs, 3H), 2.13-2.03 (m, 3H), 0.90-0.85 (m, 4H). LC MS (Method B): M+1: 396.15, tRet: 1.109 min and HPLC (Method C): tRet: 5.473 min.

### Example 9

### 4-((6-cyclopropylpyridin-3-yl)amino)-N-methyl-2-(pyrrolidin-1-yl)pyrimidine-5-carboxamide (Table 1, Compound No. 10)

Synthesis is performed similar to Second Heteroaryl Amination Reaction and Examples above using Intermediate 5 (0.075 g, 0.247 mmol, 1.0 eq), pyrrolidine (5.0 eq.), BuOH (3 mL) and K₂CO₃ (5.0 eq) and stirring for 1.5 h at at 150° C. under microwave irradiation. Desired compound is a white solid (0.045 g, 54%). ¹H NMR (400 MHz, DMSO-d₆) δ ppm=11.44 (d, J=8.8 Hz, 1H), 8.70 (d, J=16.4 Hz, 1H), 8.56 (s, 1H), 8.40 (d, J=3.6 Hz, 1H), 8.09-7.97 (m, 2H), 7.23 (d, J=8.4 Hz, 1H), 3.75-3.67 (m, 2H), 3.56-3.45 (m, 2H), 3.05-2.99 (m, 1H), 2.76 (d, J=4.0 Hz, 3H), 2.60 (bs, 3H), 2.13-2.03 (m, 3H), 0.90-0.85 (in, 4H). LC-MS (Method B): M+1: 338.10, tRet: 1.160 min and HPLC (Method B): tRet: 5.240 min.

### 4-((6-cyclopropylpyridin-3-yl)amino)-N-methyl-2-(pyrrolidin-1-yl)pyrimidine-5-carboxamide Hydrochloride

A solution of 4-((6-cyclopropylpyridin-3-yl)amino)-N-methyl-2-(pyrrolidin-1-yl) pyrimidine-5-carboxamide (0.030 g, 0.088 mmol, 1.0 eq.) in 4M HCl in dioxane (2 mL) is stirred for 2 h at 27° C. The reaction mixture is evaporated completely to afford the desired compound (0.020 g, 60%) as a white solid. ¹H NMR (400 MHz, DMSO-d₆) δ ppm: 6=12.14 (s, 1H), 9.20 (bm 2H), 8.79 (s, 1H), 8.41 (d, J=8.0 Hz, 1H), 7.60 (d, J=8.8 Hz, 1H), 3.62 (t, J=6.8 Hz, 4H), 2.79 (d, J=4.4 Hz, 3H), 2.37-2.32 (m, 1H), 2.04-1.94 (bm, 4H), 1.25-1.23 (m, 2H), 1.14-1.13 (m, 2H). LC-MS (Method B): (M−HCl)+1: 339.10, tRet: 1.880 min and HPLC (Method B): tRet: 5.207 min.

### Example 10

### (S)-4-((6-cyclopropylpyridin-3-yl)amino)-N-methyl-2-(3-(methylcarbamoyl)pyrrolidin-1-yl)pyrimidine-5-carboxamide (Table 1, Compound No. 6)

Synthesis is performed similar to Second Heteroaryl Amination Reaction and Examples above using Intermediate 5 (100.0 mg, 0.329 mmol, 1.0 eq), (S)—N-methylpyrrolidine-3-carboxamide hydrochloride (1.5 eq), n-butanol (6.0 mL), and DIPEA (8.0 eq) and stirring for 16 h at 150° C. Desired compound is an off-white solid (30.0 mg, 23%). LC-MS: M−1: 394.10, tRet=1.10; HPLC: tRet=5.00. ¹H NMR (400 MHz, DMSOd₆): δ=11.44-11.42 (1H, J=9.2 Hz, 4H), 8.72-8.68 (m, 1H), 8.56 (s, 1H), 8.40-8.39 (m, 1H), 8.07-8.01 (m, 2H), 7.25-7.229 (d, J=8.4 Hz, 1H), 3.54-3.47 (m, 2H), 3.42-3.31 (m, 3H), 3.03 (m, 1H), 2.770-2.759 (d, J=4.4 Hz, 3H), 2.67-2.59 (m, 4H), 2.2-2.0 (m, 3H), 0.90-0.84 (m, 4H).

### Example 11

### 7-methoxy-N-(5-(methylcarbamoyl)-2-(3-(methylcarbamoyl)pyrrolidin-1-yl)pyrimidin-4-yl)-1H-indazole-3-carboxamide (Table 1, Compound No. 56)

To a stirred solution of N-(2-chloro-5-(methylcarbamoyl)pyrimidin-4-yl)-7-methoxy-1H-indazole-3-carboxamide (0.050 g, 0.138 mmol, 1.0 eq.) in 5.0 mL of N,N′-dimethylacetamide is added DIPEA (0.12 mL, 0.69 mmol, 5.0 eq.) and N-methylpyrrolidine-3-carboxamide hydrochloride (racemic) (0.034 g, 0.208 mmol, 1.5 eq.) and then heated at 100° C. in a sealed tube for 16 h. The reaction mixture turns brown solution. The progress of the reaction is monitored by TLC. The reaction mixture is quenched with 20 mL of water and extracted with (3×30 mL) of EtOAc. The combined organic layers are washed with (4×50 mL) of brine solution, dried over anhydrous sodium sulphate and evaporated to dryness to afford the crude compound. The crude compound is purified by preparative HPLC. Collected fractions are dried under lyophilization for 24 h to afford the desired compound as an off-white solid. Yield: 0.020 g (32%). ¹H-NMR (400 MHz, DMSO-d6): δ=14.1 (s, 1H), 13.17 (d, J=7.2 Hz, 1H), 8.70 (s, 1H), 8.49 (d, J=4.0 Hz, 1H), 8.0 (m, J=21.6 Hz, 1H), 7.77 (d, J=7.6 Hz, 1H) 7.21 (dd, J=7.6, 8.0 Hz, 1H), 6.94 (d, J=7.6 Hz, 1H), 3.99 (s, 3H), 3.90-3.74 (m, 2H), 3.6-3.4 (m, 2H), 3.03 (m, 1H), 2.78 (d, J=4 Hz, 3H), 2.62 (m, 3H), 2.1 (m, 2H) and LC-MS (Method B): [M+H]*=453.15; [2M+H]⁺=905.30; [M−H]⁺=451.10; [2M−H]⁺=903.25; tRet=1.21 min; HPLC (Method B): tRet=5.907 min (210 nm); tRet=5.907 min (255 nm).

### Example 12

### N-methyl-4-((4-(morpholine-4-carbonyl)phenyl)amino)-2-(pyrrolidin-1-yl)pyrimidine-5-carboxamide (Table 1, Compound No. 8)

To a stirred solution of 2-chloro-N-methyl-4-((4-(morpholine-4-carbonyl)phenyl)amino)pyrimidine-5-carboxamide (0.100 g, 0.266 mmol, 1.0 eq.) in BuOH (3 mL) is added pyrrolidine (0.095 g, 1.330 mmol, 5.0 eq) and K₂CO₃ (0.184 g, 1.330 mmol, 5.0 eq.). Then the reaction mixture is stirred for 1.5 h at 150° C. in microwave irradiation. Completion of the reaction is monitored by TLC. After completion of the reaction, the reaction mixture is evaporated to afford the crude compound. The crude material is purified by flash chromatography using 3% MeOH in DCM as an eluent to afford the desired compound (0.030 g, 27%) as an off white solid. ¹H NMR (400 MHz, DMSO-d₆): δ ppm=11.70 (s, 1H), 8.58 (s, 1H), 8.79 (s, 1H), 8.41 (d, J=4.4 Hz, 1H), 7.83 (d, J=8.8 Hz, 2H), 7.40 (d, J=8.8 Hz, 2H), 3.60-3.40 (m, 12H), 2.77 (d, J=4.4 Hz, 3H), 1.94 (bm, 4H). LC-MS (Method B): M+1: 411.15, tRet: 1.144 min and also HPLC (Method B): tRet: 5.298 min.

### Example 13

### 4-((3-(5-fluoropyrimidin-2-yl)-2-methoxyphenyl)amino)-2-(3-(methylcarbamoyl)azetidin-1-yl)pyrimidine-5-carboxamide (Table 1, Compound No. 48)

To a stirred solution of 2-chloro-4-((3-(5-fluoropyrimidin-2-yl)-2-methoxyphenyl)amino)pyrimidine-5-carboxamide (0.07 g, 0.187 mmol, 1.0 eq) in n-BuOH (10 mL) is added N-methylazetidine-3-carboxamide hydrochloride (0.042 g, 0.280 mmol, 1.5 eq) followed by DIPEA (0.48 mL, 2.805 mmol, 15 eq). The reaction mixture turns into a clear solution and is stirred for 16 h at 150° C. The progress of the reaction is monitored by TLC. After completion of the reaction, the reaction mixture is cooled to RT and volatile solvents are removed under reduced pressure to afford the residue. The residue is diluted with water (20 mL), the aqueous layer is extracted with ethyl acetate (30 mL×3), organic phases are washed with brine (20 mL×1), dried over sodium sulphate and evaporated to dryness to afford the crude compound. The crude compound is purified by MPLC using DCM/MeOH (gradient 5-10% MeOH) to afford the desired compound as an off-white solid (45 mg, 53% yield). ¹H NMR (400 MHz, DMSO-d₆): δ=12.06 (s, 1H), 9.03 (s, 2H), 8.83 (dd, J=1.2 Hz, 8.0 Hz, 1H), 8.62 (s, 1H), 8.03-7.96 (m, 2H), 7.34-7.31 (m, 2H), 7.21 (t, J—8.0 Hz, 1H), 4.23-4.12 (m, 4H), 3.69 (s, 3H), 3.46-3.39 (m, 1H), 2.63 (d, J=4.4 Hz, 3H). LCMS (Method-B): m/z=453.15 (M+H)⁺ tRet: 1.17 min. HPLC (Method-A): tRet: 5.41 min.

### Example 14

### 4-((3-(5-fluoropyrimidin-2-yl)-2-methoxyphenyl)amino)-N-methyl-2-(3-(methylcarbamoyl)azetidin-1-yl)pyrimidine-5-carboxamide (Table 1, Compound No. 49)

To a stirred solution of 2-chloro-4-((3-(5-fluoropyrimidin-2-yl)-2-methoxyphenyl)amino)-N-methylpyrimidine-5-carboxamide (0.07 g, 0.180 mmol, 1.0 eq) in n-BuOH (10 mL) is added N-methylazetidine-3-carboxamide hydrochloride (0.040 g, 0.270 mmol, 1.5 eq) followed by DIPEA (0.47 mL, 2.700 mmol, 15 eq). Then the reaction mixture turns to a clear solution and is stirred for 16 h at 150° C. The progress of the reaction is monitored by TLC. After completion of the reaction, the reaction mixture is cooled to RT and volatile solvents are removed under reduced pressure. The obtained mass is diluted with water (20 mL), the aqueous layer is extracted with ethyl acetate (30 mL×3), organic phase is washed with brine (20 mL×1), dried over sodium sulphate and evaporated to dryness to afford the crude compound. The crude compound is purified by MPLC using DCM/MeOH (gradient 5-10% MeOH) to afford the desired compound as an off-white solid (50 mg, 59% yield). ¹H NMR (400 MHz, DMSO-d₆): 5=11.85 (s, 1H), 9.03 (s, 2H), 8.81-8.79 (m, 1H), 8.56 (s, 1H), 8.41-8.40 (m, 1H), 8.03-8.02 (m, 1H), 7.33-7.31 (m, 1H), 7.21 (t, J=8.0 Hz, 1H), 4.22-4.12 (m, 4H), 3.70 (s, 3H), 3.44-3.40 (m, 1H), 2.76 (d, J=4.4 Hz, 3H), 2.63 (d, J=4.8 Hz, 3H). LCMS (Method-B): m/z=467.15 (M+H)⁺ tRet: 1.17 min. HPLC (Method-B): tRet: 5.45 min.

### Example 15

### 4-((6-cyclopropylpyridin-3-yl)amino)-2-(3-(methylcarbamoyl)azetidin-1-yl)pyrimidine-5-carboxamide (Table 1, Compound No. 50)

Synthesis is performed similar to Second Heteroaryl Amination Reaction and Examples above using Intermediate 4 (150.0 mg, 0.517 mmol, 1.0 eq), n-butanol, N-methylazetidine-3-carboxamide hydrochloride (1.5 eq), and N,N-diisopropylethylamine (8.0 eq) and stirring for 16 h at 150° C. Desired compound is an off-white solid (75 mg, 39% yield). LCMS (Method B): m/z=368.05 (M+H)⁺, 409.10 (M+ACN+H)⁺, 366.15 (M−H)⁺, tRet: 1.09 min. HPLC (Method A): tRet: 1.09 min. ¹H NMR (400 MHz, DMSO-d₆) δ=11.54 (s, 1H), 8.67 (s, 2H), 8.03 (m, 3H), 7.63 (s, 1H), 7.24-7.22 (d, J=8.4, 1H), 4.19 (m, 4H), 3.39 (s, 2H), 2.67 (m, 3H), 2.06 (m, 1H), 1.25 (m, 4H).

### Example 16

### 4-((6-cyclopropylpyridin-3-yl)amino)-N-methyl-2-(3-(methylcarbamoyl)azetidin-1-yl)pyrimidine-5-carboxamide (Table 1, Compound No. 51)

Synthesis is performed similar to Second Heteroaryl Amination Reaction and Examples above using Intermediate 5 (150.0 mg, 0.493 mmol, 1.0 eq), n-butanol (10.0 mL), N-methylazetidine-3-carboxamide hydrochloride (1.5 eq), and N,N-diisopropylethylamine (8.0 eq) and stirring for 16 h at 150° C. Desired compound is an off-white solid (75 mg, 39% yield). LCMS (Method B): m/z=382.10 (M+H)⁺, 423.15 (M+ACN+H)⁺, 380.15 (M−H)⁺ (tRet: 1.11 min). HPLC (Method B): tRet: 5.95 min. ¹H NMR (400 MHz, DMSO-d₆): δ=11.40 (s, 1H), 8.658-8.652 (d, J=2.4 Hz, 1H), 8.54 (s, 1H), 8.44-8.43 (d, J=4.4 Hz, 1H), 8.08-8.07 (d, J=2.4 Hz, 1H), 8.06 (d, J=8.4 Hz, 1H), 8.01 (d, J=4.4 Hz, 1H), 7.23 (d, J=8.4 Hz, 1H), 4.19 (m, 4H), 3.41-3.29 (m, 1H), 2.76-2.75 (d, J=4.8 Hz, 3H), 2.67-2.66 (d, J=4.4 Hz, 3H), 2.06 (m, 1H), 0.90-0.86 (m, 4H).

### Example 17

### Example 17A

### Ethyl 1-(5-carbamoyl-4-((3-(5-fluoropyrimidin-2-yl)-2-methoxyphenyl)amino)pyrimidin-2-yl)piperidine-4-carboxylate (Table 1, Compound No. 39)

To a stirred solution of 2-chloro-4-((3-(5-fluoropyrimidin-2-yl)-2-methoxyphenyl)amino)pyrimidine-5-carboxamide (0.22 g, 0.588 mmol, 1.0 eq) in n-BuOH (12 mL) is added ethyl piperidine-4-carboxylate (0.135 mL, 0.882 mmol, 1.5 eq) followed by DIPEA (1.53 mL, 8.821 mmol, 15 eq). The reaction mixture turns into a clear solution and is stirred for 16 h at 150° C. The progress of the reaction is monitored by TLC. After completion of the reaction, the reaction mixture is cooled to RT and volatile solvents are removed under reduced pressure then the obtained mass is diluted with water (30 mL), the aqueous layer is extracted with ethyl acetate (30 mL×2), organic phases are washed with brine (30 mL×1), dried over anhydrous sodium sulphate and evaporated to dryness to afford the crude compound. The crude compound is purified by MPLC using EtOAc to afford the desired compound as an off-white solid (0.21 g, 72% yield). ¹H NMR (400 MHz, DMSO-d₆): δ=12.14 (s, 1H), 9.18 (s, 2H), 8.80 (s, 1H), 8.77-8.73 (m, 1H), 8.09 (bs, 1H), 7.55-7.38 (m, 3H), 4.72-4.60 (m, 2H), 4.25-4.20 (m, 2H), 3.82 (s, 3H), 3.31 (t, J=12 Hz, 2H), 2.84-2.81 (m, 1H), 2.13-2.07 (m, 2H), 1.73-1.65 (m, 2H), 1.34 (t, J=7.2 Hz, 3H). LCMS (Method-B): m/z=496.25 (M+H)⁺, tRet: 1.35 min. HPLC (Method-A): tRet: 6.66 min.

### Example 17B

### 1-(5-carbamoyl-4-((3-(5-fluoropyrimidin-2-yl)-2-methoxyphenyl)amino)pyrimidin-2-yl)piperidine-4-carboxylic Acid (Table 1, Compound No. 38)

To a stirred solution of ethyl 1-(5-carbamoyl-4-((3-(5-fluoropyrimidin-2-yl)-2-methoxyphenyl)amino)pyrimidin-2-yl)piperidine-4-carboxylate (0.15 g, 0.302 mmol, 1.0 eq) in THE (8 mL) and H₂O (2 mL) is added LiOH·H₂O (0.063 g, 1.514 mmol, 5.0 eq). The reaction turns into a clear solution and is stirred for 16 h at RT. The progress of the reaction is monitored by TLC. After completion of the reaction, volatile solvents are removed under reduced pressure, then the obtained mass is diluted with water (30 mL), and acidified with 1N HCl to pH=5 to 6. The solid formed is filtered and dried under vacuum to afford the desired compound as an off-white solid (0.12 g, 85% yield). ¹H NMR (400 MHz, DMSO-d₆): δ=12.26 (bs, 1H), 11.98 (s, 1H), 9.03 (s, 2H), 8.65 (s, 1H), 8.60 (d, J=8 Hz, 1H), 7.94 (bs, 1H), 7.35-7.23 (m, 3H), 4.54 (d, J=12 Hz, 2H), 3.69 (s, 3H), 3.16 (t, J=11.6 Hz, 2H), 2.61-2.56 (m, 1H), 1.92 (d, J=11.2 Hz, 2H), 1.57-1.52 (m, 2H). LCMS (Method B): m/z=468.15 (M+H)⁺, tRet: 1.21 min, HPLC (Method-A): tRet: 5.74 min.

### Example 18

### Example 18A

### Ethyl 1-(4-((3-(5-fluoropyrimidin-2-yl)-2-methoxyphenyl)amino)-5-(methylcarbamoyl)pyrimidin-2-yl)piperidine-4-carboxylate (Table 1, Compound No. 28)

To a stirred solution of 2-chloro-4-((3-(5-fluoropyrimidin-2-yl)-2-methoxyphenyl)amino)-N-methylpyrimidine-5-carboxamide (90 mg, 0.23 mmol, 1.0 eq) in n-butanol (5 mL) in a sealed tube are added DIPEA (196.76 μL, 1.15 mmol, 5.0 eq) and ethyl piperidine-4-carboxylate (53.51 μL, 0.34 mmol, 1.5 eq) at RT. The reaction mixture is heated at 140° C. for 16 h. The progress of the reaction is monitored by TLC. The reaction mixture is concentrated to dryness. The residue is dissolved in ethyl acetate (50 mL), washed with water (10 mL) and brine (10 mL), dried over anhydrous Na₂SO₄ and concentrated and dried to afford the crude compound. The crude compound is purified by preparative HPLC. The collected fractions are lyophilized for 48 h to afford the desired compound as an off-white solid (55 mg, 46% yield). ¹H NMR (400 MHz, DMSO-d₆) δ 11.76 (s, 1H), 9.03 (s, 2H), 8.58-8.55 (m, 2H), 8.38-8.37 (m, 1H), 7.35-7.33 (m, 1H), 7.27-7.23 (m, 1H), 4.56-4.53 (m, 2H), 4.10-4.05 (m, 2H), 3.70 (s, 3H), 3.15 (t, J=11.6 Hz, 2H), 2.78 (d, J=8.0 Hz, 3H), 2.71-2.65 (m, 1H), 1.94-1.92 (m, 2H), 1.58-1.50 (m, 2H), 1.20 (t, J=12.0 Hz 3H). LCMS (Method A): m/z=510.3 (M+H)⁺, tRet: 2.37 min. HPLC (Method A): tRet: 7.40 min.

### Example 18B

### 1-(4-((3-(5-fluoropyrimidin-2-yl)-2-methoxyphenyl)amino)-5-(methylcarbamoyl)pyrimidin-2-yl)piperidine-4-carboxylic acid (Table 1, Compound No. 27)

To a stirred solution of ethyl 1-(4-((3-(5-fluoropyrimidin-2-yl)-2-methoxyphenyl)amino)-5-(methylcarbamoyl)pyrimidin-2-yl)piperidine-4-carboxylate (80 mg, 0.15 mmol, 1.0 eq) in THE (5 mL) and water (1 mL) is added LiOH·H₂O (65.94 mg, 10.0 eq) at RT. The reaction mixture is stirred at RT for 16 h. The progress of the reaction is monitored by TLC. The reaction mixture is concentrated. The residue is taken in water (5 mL) and washed with ethyl acetate (10 mL). The aqueous part is acidified with 1N aq. HCl (pH=2). The obtained solid is filtered and dried to afford the desired compound as an off-white solid (60 mg, 80% yield). ¹H NMR (400 MHz, DMSO-d₆) δ 12.20 (s, 1H), 9.03 (s, 2H), 8.86 (bs, 1H), 8.63 (s, 1H), 8.45 (d, J=8.0 Hz, 1H), 7.48-7.46 (m, 1H), 7.31 (t, J=8.0 Hz, 1H), 4.43 (d, J=13.2 Hz, 2H), 3.70 (s, 3H), 3.29 (t, J=12.0 Hz, 2H), 2.78 (d, J=4.4 Hz, 3H), 2.67-2.61 (m, 1H), 1.98-1.95 (m, 2H), 1.67-1.58 (m, 2H). LCMS (Method B): m/z=482.25 (M+H)⁺ (tRet: 1.21 min). HPLC (Method A): tRet: 5.55 min.

### Example 19

### Example 19A

### Ethyl 1-(5-carbamoyl-4-((6-cyclopropylpyridin-3-yl)amino)pyrimidin-2-yl)piperidine-4-carboxylate (Table 1, Compound No. 36)

Synthesis is performed similar to Second Heteroaryl Aminiation Reaction and Examples above using Intermediate 4 (1.0 eq), ethyl piperidine-4-carboxylate (5.0 eq), and N,N-diisopropylethylamine in a sealed tube and stirring for 16 h at 115° C. Desired compound is an off-white solid. Yield: 140.0 mg, 40%. LC-MS: M−1: 409.15, tRet=1.21, HPLC: tRet=5.74. ¹H NMR (400 MHz, DMSOd₆) δ=11.45 (s, 1H), 8.6 (s, 1H), 8.589-8.583 (d, J=2.4 Hz, 1H), 7.95-7.90 (m, 2H), 7.371-7.25 (m, 2H), 4.48 (s, 2H), 4.10-4.03 (q, 2H), 3.12-3.06 (m, 2H), 2.67-2.64 (m, 1H), 2.07-2.04 (m, 1H), 1.91-1.88 (m, 2H), 1.54-1.45 (m, 2H), 1.20-1.51 (t, 3H), 0.91-0.856 (m, 4H).

### Example 19B

### 1-(5-carbamoyl-4-((6-cyclopropylpyridin-3-yl)amino)pyrimidin-2-yl)piperidine-4-carboxylic acid (Table 1, Compound No. 35)

Synthesis is performed similar to Saponification Reaction and Examples above using ethyl 1-(5-carbamoyl-4-((6-cyclopropylpyridin-3-yl)amino)pyrimidin-2-yl)piperidine-4-carboxylate (1.0 eq) in tetrahydrofuran and lithium hydroxide monohydrate (5.0 eq) in water. Reaction mixture is stirred at RT for 16 h. Desired compound is an off-white solid. Yield: 30.0 mg, 32%. LC-MS: M+1: 383.05, tRet=1.13, HPLC: tRet=5.17. ¹H NMR (400 MHz, DMSOd₆) δ=12.27 (1H, s), 11.44 (1H, s), 8.62 (1H, s), 8.589-8.583 (d, J=2.4 Hz, 1H), 7.933-7.90 (m, 2H), 7.33-7.25 (m, 2H), 4.48 (m, 2H), 3.125-3.06 (m, 2H), 2.07-2.04 (m, 1H), 1.906-1.873 (m, 2H), 1.53-1.43 (m, 2H), 0.917-0.856 (m, 4H).

### Example 20

### Example 20A

### Ethyl 1-(4-((6-cyclopropylpyridin-3-yl)amino)-5-(methylcarbamoyl)pyrimidin-2-yl)piperidine-4-carboxylate (Table 1, Compound No. 20)

Synthesis is performed similar to Second Heteroaryl Amination Reaction and Examples above using Intermediate 5 (1.0 eq), BuOH, ethyl piperidine-4-carboxylate (3.0 eq), and DIPEA (5.0 eq) and stirring for 1.5 h at 150° C. under microwave irradiation. Desired compound is an off white solid (300 mg, 53% yield). LCMS (Method B): m/z=425.15 (M+H)⁺ tRet: 1.15 min; HPLC (Method B): tRet: 5.288 min. ¹H NMR (400 MHz, DMSO-d₆) δ=11.34 (s, 1H), 8.58 (m, 2H), 8.41 (d, J=4.4 Hz, 1H), 7.94 (dd, J₁=8.8 Hz, J₂=2.8 Hz, 2H), 7.25 (d, J=8.4 Hz, 1H), 4.48 (bs, 2H), 4.07 (q, J=7.2 Hz, 2H), 3.09 (t, J=11.2 Hz, 2H), 2.76 (d, J=4.4 Hz, 3H), 2.67-2.62 (m, 1H), 2.08-2.04 (m, 1H), 1.91-1.88 (m, 2H), 1.55-1.46 (m, 2H), 1.18 (t, J=7.2 Hz, 3H), 0.91-0.85 (m, 4H).

### Example 20B

### 1-(4-((6-cyclopropylpyridin-3-yl)amino)-5-(methylcarbamoyl)pyrimidin-2-yl)piperidine-4-carboxylic acid (Table 1, Compound No. 19)

Synthesis is performed similar to Saponification Reaction and Examples above using ethyl 1-(4-((6-cyclopropylpyridin-3-yl)amino)-5-(methylcarbamoyl)pyrimidin-2-yl)piperidine-4-carboxylate (1.0 eq) in THF, MeOH, water (2:1:1), and lithium hydroxide·H₂O (5.0 eq) and stirring for 16 h at RT. Desired compound is an off-white solid (30 mg, 21% yield). LCMS Method B: m/z=397.10 (M+H)⁺. ¹H NMR (400 MHz, DMSO-d₆) δ=12.27 (bs, 1H), 11.33 (s, 1H), 8.58-8.56 (m, 2H), 8.41 (d, J=4.4 Hz, 1H), 7.94 (dd, J₁=8.8 Hz, J₂=2.8 Hz, 2H), 7.25 (d, J=8.4 Hz, 1H), 4.48 (bs, 2H), 3.09 (t, J=11.2 Hz, 2H), 2.76 (d, J=4.4 Hz, 3H), 2.66 (m, 1H), 2.08-2.04 (m, 1H), 1.90-1.87 (bm, 2H), 1.52-1.43 (bm, 2H), 0.92-0.85 (bm, 4H).

### Example 21

### Example 21A

### Ethyl 1-(5-carbamoyl-4-((2-methoxyphenyl)amino)pyrimidin-2-yl)piperidine-4-carboxylate (Table 1, Compound No. 45)

To a stirred solution of 2-chloro-4-((2-methoxyphenyl)amino)pyrimidine-5-carboxamide (180.0 mg, 0.645 mmol, 1.0 eq) in n-butanol (10.0 mL) in a sealed tube are added ethyl piperidine-4-carboxylate (152.0 mg, 0.968 mmol, 1.5 eq) and N,N-diisopropylethylamine (0.95 mL, 5.166 mmol, 8.0 eq) at RT. The reaction mixture is heated at 150° C. for 16 h. The progress of the reaction is monitored by TLC. The reaction mixture turns to a light yellow solution. The reaction mixture is concentrated under reduced pressure, then it is dissolved in ethyl acetate (50 mL), washed with water (2×50 mL), dried over anhydrous Na₂SO₄ and concentrated to dryness to afford the crude compound. The crude compound is purified by flash chromatography, eluting with 5% MeOH in DCM. The fractions containing the product are concentrated under reduced pressure and dried completely to afford 130 mg of compound which is again purified by preparative HPLC. The collected fractions are lyophilied for 48 h to afford the required compound as an off-white solid (40 mg, 15%, yield). LCMS (Method B): m/z=400.10 (M+H)⁺ (tRet: 1.31 min). HPLC (Method A): tRet: 1.31 min. ¹H NMR (400 MHz, DMSO-d₆) δ=11.62 (s, 1H), 8.06 (s, 1H), 8.38 (d, J=7.6 Hz, 1H), 7.05-6.94 (m, 4H), 4.53 (d, J=10.4 Hz, 2H), 4.08-4.06 (m, 2H), 4.04 (s, 3H), 3.15-3.09 (m, 2H), 2.67-2.52 (m, 1H), 1.89 (m, 2H), 1.52-1.50 (m, 3H).

### Example 21B

### 1-(5-carbamoyl-4-((2-methoxyphenyl)amino)pyrimidin-2-yl)piperidine-4-carboxylic Acid (Table 1, Compound No. 44)

To a stirred solution of ethyl 1-(5-carbamoyl-4-((2-methoxyphenyl)amino)pyrimidin-2-yl)piperidine-4-carboxylate (50 mg, 0.125 mmol, 1.0 eq) in THF (5 mL) and water (0.2 mL) is added LiOH·H₂O (26 mg, 0.625 mmol, 5.0 eq) at RT. The reaction mixture is stirred at RT for 16 h. The progress of the reaction is monitored by TLC. The reaction mixture is concentrated to remove THF. The obtained residue is taken in water (5 mL) and washed with ethyl acetate (10 mL). The aqueous part is acidified with 1N aq. HCl (pH=2). The obtained solid is filtered and dried completely to afford the desired compound as a pale brown solid (30 mg, 65% yield). LCMS (Method A): m/z=372.2 (M+H)⁺ (tRet: 2.01 min), HPLC (Method A): tRet: 5.63 min. ¹H NMR (400 MHz, DMSO-d₆) δ=12.30 (s, 1H), 11.65 (s, 1H), 8.62-8.60 (d, J=10 Hz, 1H), 8.38-8.36 (d, J=6.8 Hz, 1H), 7.94 (m, 1H), 7.06-6.95 (m, 4H), 4.51-4.48 (d, J=11.2 Hz, 2H), 3.87 (s, 3H), 3.16 (m, 2H), 2.57 (m, 1H), 1.91 (m, 2H), 1.54 (m, 2H).

### Example 22

### Example 22A

### Ethyl 1-(4-((2-methoxyphenyl)amino)-5-(methylcarbamoyl)pyrimidin-2-yl)piperidine-4-carboxylate (Table 1, Compound No. 34)

Synthesis is performed similar to Second Heteroaryl Amination Reaction and Examples above using 2-chloro-4-((2-methoxyphenyl)amino)-N-methylpyrimidine-5-carboxamide (1.0 eq.), 1-butanol, ethyl piperidine-4-carboxylate (1.5 eq), and DIPEA (3.0 eq) and heating to 100° C. for 16 h. Desired compound is an off-white solid. Yield: 0.080 g (38%). LC-MS (Method B): [M+1]+=414.25; tRet=1.33 min; HPLC (Method B): tRet=6.490 min.

### Example 22B

### 1-(4-((2-methoxyphenyl)amino)-5-(methylcarbamoyl)pyrimidin-2-yl)piperidine-4-carboxylic Acid (Table 1, Compound No. 33)

Synthesis is performed similar to Saponification Reaction and Examples above using ethyl 1-(4-((2-methoxyphenyl)amino)-5-(methylcarbamoyl)pyrimidin-2-yl)piperidine-4-carboxylate (1.0 eq), THF, lithium hydroxide monohydrate (5.0 eq), and water. Desired compound is an off-white solid. Yield: 0.090 g (80%). ¹H-NMR (400 MHz, DMSO-d₆): δ 12.3 (brs, 1H), 11.4 (s, 1H) 8.5 (s, 1H), 8.37 (d, J=7.6 Hz, 1H), 8.3 (d, J=4.4 Hz, 1H), 6.93-7.06 (m, 3H), 4.52-4.50 (m, 2H), 3.88 (s, 3H), 3.11 (t, J=11.4 Hz, 2H), 2.78 (d, J=7.1 Hz, 3H), 2.5 (m, 1H), 1.88-1.91 (m, 2H), 1.46-1.54 (m, 2H) LC-MS (Method A): [M+1]+=386.2; tRet=2.041 min; HPLC (Method B): tRet=5.723 min.

### Example 23

### Example 23A

### Ethyl 1-(5-carbamoyl-4-((2-(methylsulfonyl)phenyl)amino)pyrimidin-2-yl)piperidine-4-carboxylate (Table 1, Compound No. 16)

To a stirred solution of 2-chloro-4-((2-(methylsulfonyl)phenyl)amino)pyrimidine-5-carboxamide (0.120 g, 0.367 mmol, 1.0 eq) in n-BuOH (5 mL), ethyl piperidine-4-carboxylate (0.173 g, 1.102 mmol, 3.0 eq) and DIPEA (0.192 mL, 1.102 mmol, 3.0 eq) are added. Then the reaction mixture is stirred for 1.5 h at 150° C. in microwave irradiation. Reaction mixture turns into a clear solution. Completion of the reaction is monitored by TLC. After completion of the reaction, the reaction mixture is evaporated to afford the crude compound. The crude compound is purified by preparative HPLC. After preparative HPLC purification, the compound is lyophilized for 48 h Then the solid is dissolved in 30 mL 10% MeOH in DCM, washed with saturated aq. NaHCO₃ solution, dried over sodium sulphate, concentrated and lyophilized for 24 h to afford the desired compound as an off white solid (13 mg, 8% yield). LCMS (Method A): m/z=448.2 (M+H) tRet: 2.264, 446.1 (M−H). HPLC (Method B): tRet: 6.492 min. ¹H NMR (400 MHz, DMSO-d₆): δ=11.61 (s, 1H), 8.67 (s, 1H), 8.19 (d, J=8.4 Hz, 1H), 7.90 (d, J=6.8 Hz, 2H), 7.69 (t, J=7.6 Hz, 1H), 7.32 (d, J=7.6 Hz, 2H), 4.46 (brs, 2H), 4.06 (q, J=6.8 Hz, 2H), 3.16 (s, 3H), 3.12-3.01 (brm, 2H), 2.67-2.62 (m, 1H), 1.87 (d, J=10.8 Hz, 2H), 1.53-1.44 (m, 2H), 1.17 (t, J=14.4 Hz, 3H).

### Example 23B

### 1-(5-carbamoyl-4-((2-(methylsulfonyl)phenyl)amino)pyrimidin-2-yl)piperidine-4-carboxylic Acid (Table 1, Compound No. 78)

To a stirred solution of ethyl 1-(5-carbamoyl-4-((2-(methylsulfonyl)phenyl)amino)pyrimidin-2-yl)piperidine-4-carboxylate (0.120 g, 0.27 mmol, 1.0 eq.) in THF:MeOH:H₂O (6.0 mL) is added lithium hydroxide monohydrate (0.056 g, 1.34 mmol, 5.0 eq). The reaction mixture is stirred for 4 h at RT. The reaction mixture turns to white precipitate. The reaction is monitored by TLC. After completion of the reaction, the solvent is concentrated under reduced pressure to afford the crude compound. The crude compound is diluted with water (10 mL), acidified with 1N HCl and extracted with 10% MeOH in DCM (3×50 mL). The combined organic layers are dried over Na₂SO₄ and evaporated to afford the desired compound as an off white solid (80 mg, 71% yield). LCMS: m/z=420.05 (M+H), 418.05 (M−H) (Method B). ¹H NMR (400 MHz, DMSO-d₆) δ=10.49 (d, J=4.0 Hz, 1H), 8.48 (s, 1H), 8.42 (s, 1H), 8.11 (d, J=8.4 Hz, 1H), 7.63 (d, J=6.8 Hz, 1H), 7.24 (d, J=6.8 Hz, 1H), 6.62 (t, J=7.6 Hz, 1H), 6.40 (d, J=4.4 Hz, 1H), 4.45 (d, J=12.8 Hz, 2H), 3.11 (s, 3H), 2.79 (t, J=11.2 Hz, 2H), 2.03 (t, J=10.8 Hz, 1H), 1.68 (d, J=10.8 Hz, 2H), 1.45-1.37 (m, 2H).

### Example 24

### Example 24A

### Ethyl 1-(5-(methylcarbamoyl)-4-((2-(methylsulfonyl)phenyl)amino)pyrimidin-2-yl)piperidine-4-carboxylate (Table 1, Compound No. 17)

Synthesis is performed similar to Second Heteroaryl Amination Reaction and Examples above using Intermediate 9 (1.0 eq), n-BuOH (5 mL), ethyl piperidine-4-carboxylate (5.0 eq), and DIPEA (5.0 eq) and stirring for 1.5 h at 150° C. in microwave irradiation. Product is purified by preparative HPLC. After preparative HPLC, compound is lyophilized for 72 h. Desired product is an off white solid (0.030 g, 12%). ¹H NMR (400 MHz, DMSO-d₆): δ ppm=11.44 (s, 1H), 8.61 (s, 1H), 8.38 (bm, 1H), 8.14 (d, J=8.4 Hz, 1H), 7.89-7.88 (bm, 1H), 7.71-7.67 (bm, 1H), 7.34-7.32 (bm, 1H), 4.45 (bm, 2H), 4.06 (q, J=6.8 Hz, 2H), 3.16 (s, 3H), 3.05-3.02 (bm, 2H), 2.75 (d, J=4.4 Hz, 3H), 1.86-1.81 (m, 3H), 1.52-1.46 (m, 2H), 1.17 (t, J=7.2 Hz, 3H). LC-MS (Method A): M+1: 462.2, M−1: 460.2, tRet: 2.314 min and HPLC (Method B): tRet: 6.628 min.

### Example 24B

### 1-(5-(methylcarbamoyl)-4-((2-(methylsulfonyl)phenyl)amino)pyrimidin-2-yl)piperidine-4-carboxylic Acid (Table 1, Compound No. 68)

Synthesis is performed similar to Saponification Reaction and Examples above using ethyl 1-(5-(methylcarbamoyl)-4-((2-(methylsulfonyl)phenyl)amino)pyrimidin-2-yl)piperidine-4-carboxylate (1.0 eq), THF:MeOH:H₂O, lithium hydroxide monohydrate (5.0 eq) and stirring for 2 h at rt. After work-up, product is further purified by preparative HPLC. After preparative HPLC, purified product is lyophilized for 48 h. Desired product is a white solid (0.040 g, 60%). ¹H NMR (400 MHz, DMSO-d₆) δ ppm=11.44 (s, 1H), 8.61 (s, 1H), 8.37 (bs, 1H), 8.14 (d, J=8.4 Hz, 1H), 7.89 (d, J=6.8 Hz, 1H), 7.68 (t, J=7.6 Hz, 1H), 7.16 (t, J=7.6 Hz, 1H), 4.42 (bs, 2H), 3.20 (s, 3H), 3.05-2.99 (m, 2H), 2.75 (d, J=4.4 Hz, 3H), 2.46-2.44 (m, 1H), 1.84-1.81 (m, 2H), 1.50-1.41 (m, 2H). LCMS: M+1: 434.2, M−1: 432.0, tRet: 1.185 min.

### Example 25

### Example 25A

### Tert-Butyl 4-(5-carbamoyl-4-((3-(5-fluoropyrimidin-2-yl)-2-methoxyphenyl)amino)pyrimidin-2-yl)-3-methylpiperazine-1-carboxylate (Table 1, Compound No. 37)

2-chloro-4-((3-(5-fluoropyrimidin-2-yl)-2-methoxyphenyl)amino)pyrimidine-5-carboxamide (0.17 g, 0.454 mmol, 1.0 eq), (Rac) tert-butyl 3-methylpiperazine-1-carboxylate (0.110 mL, 0.545 mmol, 1.2 eq), N,N-diisopropylethylamine (0.38 mL, 2.27 mmol, 5.0 eq) and n-butanol (5.0 mL) in a sealed tube are stirred at 130° C. for 16 h. Color of the reaction mixture changes to amber color solution. Completion of the reaction is monitored by TLC. The reaction mixture is diluted with ethyl acetate (100.0 mL) and washed with water (100.0 mL×3 times), extracted and separated. Organic layer is dried over anhydrous sodium sulfate and concentrated to afford the crude compound. The crude compound is purified by flash chromatography, eluting with 12% methanol in DCM, purified by using preparative HPLC, and then lyophilized for two days to afford the compound as white solid. Yield: 0.12 g, 49.0%. ¹H NMR (400 MHz) V.T. NMR at 90° C. DMSOd₆ δ=11.775 (1H, s), 8.94 (s, 2H), 8.65 (s, 1H), 8.55-8.53 (m, 1H), 7.37-7.35 (m, 3H), 7.25-7.21 (m, 1H), 4.84 (m, 1H), 4.43-4.40 (m, 1H), 3.95-3.92 (m, 1H), 3.83-3.80 (m, 1H), 3.24-3.14 (m, 2H), 2.99-2.98 (m, 1H), 1.47 (s 9H), 1.18-1.17 (d, J=4.0 Hz, 3H). HPLC 210 nm tRet=7.376 (Method-B). LC-MS (Method-A): (M−1): 537.3, tRet=2.58, [M+1]+: 539.3, tRet=2.58.

### Example 25B

### 4-((3-(5-fluoropyrimidin-2-yl)-2-methoxyphenyl)amino)-2-(2-methylpiperazin-1-yl)pyrimidine-5-carboxamide (Table 1, Compound No. 57)

Acetyl chloride (0.265 mL, 3.715 mmol, 20.0 eq) is added to methanol (10.0 mL) at 0° C. slowly, stirred for 30 min at the same temperature, then (Racemic) tert-butyl 4-(5-carbamoyl-4-((3-(5-fluoropyrimidin-2-yl)-2-methoxyphenyl)amino)pyrimidin-2-yl)-3 methylpiperazine-1-carboxylate (0.1 g, 0.185 mmol, 1.0 eq) in methanol (5.0 mL) is added slowly. The mixture is stirred for 30 min at 0° C. and at RT for 5 h. Color of the reaction mixture changes to light yellow color solution. Completion of the reaction is monitored by TLC. Solvents are evaporated to afford the crude compound. The crude compound is washed with n-hexane (3.0 mL×2) times, decanted and dried to afford the pure compound which is lypholized for 2 days to afford the desired compound as an off-white solid yield: 85.0 mg, 97%. ¹H NMR DMSOd₆ (400 MHz) DMSOd₆+D₂O Exchange (400 MHz) δ=12.07 (1H, s), 9.31 (brs, 1H), 9.04 (brs, 2H), 8.93 (brs, 1H), 8.72 (m, 1H), 8.55-8.53 (m, 1H), 8.06 (brs, 1H), 7.46 (brs, 1H), 7.39-7.37 (m, 1H), 7.28 (m, 1H), 5.05 (m, 1H), 4.07 (m, 1H) 3.56 (s, 1H), 3.37-3.30 (m, 8H), 1.35 (d, 3H). HPLC (Method B): tRet=5.22, LC-MS (Method B): Parent Mol. Wt.: 438.47 [M+1]+: 439.15, tRet=1.14.

### Example 26

### 2-(4-acetyl-2-methylpiperazin-1-yl)-4-((3-(5-fluoropyrimidin-2-yl)-2-methoxyphenyl) amino)pyrimidine-5-carboxamide (Table 1, Compound No. 60)

To a stirred solution of 4-((3-(5-fluoropyrimidin-2-yl)-2-methoxyphenyl)amino)-2-(2-methylpiperazin-1-yl)pyrimidine-5-carboxamide hydrochloride (Racemic) (75.0 mg, 0.157 mmol, 1.0 eq) in DCM (5.0 mL) is added pyridine (0.101 mL, 1.256 mmol, 8.0 eq). The reaction mixture is stirred at 0° C. for 5 min, finally acetyl chloride is added (0.022 mL, 0.315 mmol, 2.0 eq). The reaction mixture is stirred at RT for 4 h. Color of the reaction mixture changes to amber color solution. Completion of the reaction is monitored by TLC. The reaction mixture is diluted with DCM (50.0 mL) and washed with water (50.0 mL×3 times), extracted and separated. Organic layer is dried over anhydrous sodium sulfate and concentrated to afford the crude compound. The crude compound is purified by flash chromatography, eluting with 15% methanol in DCM. 65.0 mg of crude compound is purified by preparative HPLC and then lyophilized for four days to afford the desired compound as white solid. Yield: 10.0 mg, 13%. ¹H NMR (400 MHz) δ=11.78 (1H, s), 8.66 (s, 1H), 8.56-8.54 (d, J=8.0 Hz, 1H), 7.37-7.35 (m, 3H), 7.24 (m, 1H), 4.86 (m, 1H), 4.45 (m, 2H), 3.57 (s, 1H), 2.65 (m, 2H), 2.1 (s, 3H), 1.18-1.17 (d, J=8.0 Hz, 1H), HPLC (Method A): tRet=5.87; LC-MS (Method A): [M+1]: 481.2, tRet=2.08 [M−1]: 479.2, tRet=2.08.

### Example 27

### Example 27A

### Tert-Butyl 4-(4-((3-(5-fluoropyrimidin-2-yl)-2-methoxyphenyl)amino)-5-(methylcarbamoyl)pyrimidin-2-yl)-3-methylpiperazine-1-carboxylate (Table 1, Compound No. 26)

To a stirred solution of 2-chloro-4-((3-(5-fluoropyrimidin-2-yl)-2-methoxyphenyl)amino)-N-methylpyrimidine-5-carboxamide (200 mg, 0.51 mmol, 1.0 eq) in n-butanol (5 mL) in a sealed tube are added DIPEA (437.40 μL, 2.57 mmol, 5.0 eq) and tert-butyl 3-methylpiperazine-1-carboxylate (156.09 μL, 0.77 mmol, 1.5 eq) at RT. The reaction mixture is heated at 140° C. for 16 h. The progress of the reaction is monitored by TLC. The reaction mixture is concentrated to dryness. The residue is dissolved in ethyl acetate (50 mL), washed with water (10 mL), brine (10 mL), dried over anhydrous Na₂SO₄, concentrated and dried to afford the crude compound. The crude compound is purified by flash chromatography, eluting with 2% MeOH in DCM. The fractions containing the product are concentrated under reduced pressure and dried completely. Wt: 270 mg. The compound obtained is again purified by preparative HPLC. The collected fractions are lyophilized for 48 h to afford the desired compound as an off-white solid (120 mg, 42% yield). ¹H NMR (400 MHz, DMSO-d₆) δ 11.77 (s, 1H), 9.03 (s, 2H), 8.61-8.58 (m, 2H), 8.41-8.39 (m, 1H), 7.36-7.34 (m, 1H), 7.29-7.25 (m, 1H), 4.84 (bs, 1H), 4.43 (bs, 1H), 4.00 (bs, 1H), 3.84 (d, J=16.0 Hz, 1H), 3.70 (s, 3H), 3.19-2.95 (m, 3H), 2.77 (d, J=8.0 Hz, 3H), 1.47 (s, 9H), 1.16-1.15 (m, 3H). LCMS (Method A): m/z=553.3 (M+H)⁺, tRet: 1.14 min. HPLC (Method A): tRet: 5.29 min.

### Example 27B

### 4-((3-(5-fluoropyrimidin-2-yl)-2-methoxyphenyl)amino)-N-methyl-2-(2-methylpiperazin-1-yl)pyrimidine-5-carboxamide Hydrochloride (Table 1, Compound No. 25)

To a stirred solution of tert-butyl 4-(4-((3-(5-fluoropyrimidin-2-yl)-2-methoxyphenyl)amino)-5-(methylcarbamoyl)pyrimidin-2-yl)-3-methylpiperazine-1-carboxylate (100 mg, 0.18 mmol, 1.0 eq) in MeOH (5 mL) is added acetyl chloride (193.68 μL, 2.71 mmol, 15.0 eq) dropwise at 0° C. The reaction mixture is stirred at RT for 16 h. Solid formation is observed in the reaction mixture. Progress of the reaction is monitored by TLC. The reaction mixture is concentrated under reduced pressure to afford the crude compound. The crude compound is triturated with diethyl ether (15 mL), filtered and dried completely to afford the desired compound as an off-white solid (85 mg, 96% yield). ¹H NMR (400 MHz, DMSO-d₆) b 11.96 (s, 1H), 9.51 (bs, 1H), 9.12 (bs, 1H), 9.04 (s, 2H), 8.69-8.63 (m, 2H), 8.49 (d, J=8.0 Hz, 1H), 7.41-7.39 (m, 1H), 7.27 (t, J=8.4 Hz, 1H), 5.03 (bs, 1H), 4.66 (d, J=14.4 Hz, 1H), 3.70 (s, 3H), 3.37-3.20 (m, 2H), 3.02-2.99 (m, 1H), 2.77 (s, 3H), 1.36 (d, J=6.8 Hz, 3H). LCMS (Method B): m/z=Product parent M.Wt: 452.49, 453.30 (M+H)⁺, tRet+: 1.24 min. HPLC (Method B): tRet: 6.38 min.

### Example 27C

### 2-(4-acetyl-2-methylpiperazin-1-yl)-4-((3-(5-fluoropyrimidin-2-yl)-2-methoxyphenyl)amino)-N-methylpyrimidine-5-carboxamide (Table 1, Compound No. 61)

To a stirred solution of 4-((3-(5-fluoropyrimidin-2-yl)-2-methoxyphenyl)amino)-N-methyl-2-(2-methylpiperazin-1-yl)pyrimidine-5-carboxamide hydrochloride (Racemic) (75 mg, 0.15 mmol, 1.0 eq) in DCM (5 mL) are added pyridine (74.24 μL, 0.91 mmol, 6.0 eq) and acetyl chloride (21.89 μL, 0.30 mmol, 2.0 eq) dropwise at 0° C. The reaction mixture is stirred at RT for 2 h. The progress of the reaction is monitored by TLC. The reaction mixture is diluted with DCM (50 mL) washed with water (10 mL), brine (10 mL), dried over anhydrous Na₂SO₄, concentrated and dried completely to afford the product as an off-white solid (30 mg, 40% yield). ¹H NMR (400 MHz, DMSO-d₆) δ 11.67 (s, 1H), 8.98 (s, 2H), 8.62 (s, 1H), 8.56 (d, J=8.0 Hz, 1H), 8.22 (bs, 1H), 7.41 (d, J=7.6 Hz, 1H), 7.26 (t, J=8.0 Hz, 1H), 4.88 (bs, 1H), 4.45 (d, J=13.6 Hz, 1H), 4.23 (bs, 1H), 3.74 (s, 3H), 3.32-3.28 (m, 4H), 2.83 (s, 3H), 2.08 (s, 3H), 1.21 (bs, 3H). LCMS (Method B): m/z=495.25 (M+H)⁺, tRet: 1.24 min. HPLC (Method A): tRet: 6.38 min.

### Example 28

### Tert-Butyl 4-(S-carbamoyl-4-((6-cyclopropylpyridin-3-yl)amino)pyrimidin-2-yl)-3-methylpiperazine-1-carboxylate (Table 1, Compound No. 13)

Synthesis is performed similar to Second Heteroaryl Amination Reaction and Examples above using Intermediate 4 (1.0 eq.), n-BuOH (6 mL), (racemic) tert-butyl 3-methylpiperazine-1-carboxylate (4.0 eq.), and DIPEA (10.0 eq.) and stirring for 1.5 h at 150° C. in microwave irradiation. Desired compound is a white solid (0.5 g, 63.93% yield). LCMS: m/z=454.20 (M+H)⁺, tRet: 1.295 min, HPLC: tRet: 6.121 min. ¹H NMR (400 MHz, DMSO-d₆) δ 11.48 (s, 1H), 8.63 (bs, 1H), 8.54 (d, J=2.4 Hz, 1H), 7.97 (dd, J₁=8.4 Hz, J₂=2.4 Hz, 2H), 7.37 (bs, 1H), 7.27 (d, J=8.4 Hz, 1H), 4.78 (bs, 1H), 4.35 (bs, 1H), 3.95 (bs, 1H), 3.80 (d, J=13.2 Hz, 1H), 3.14-2.89 (m, 3H), 2.08-2.03 (m, 1H), 1.58-1.38 (m, 9H), 1.11 (d, J=6.4 Hz, 3H), 0.94-0.86 (m, 4H).

### Example 29

4-((6-cyclopropylpyridin-3-yl)amino)-2-(2-methylpiperazin-1-yl)pyrimidine-5-carboxamide (Table 1, Compound No. 12)

Synthesis is performed similar to N-t-Boc Deprotection and Examples above using acetyl chloride (7.0 eq.), MeOH, and racemic tert-butyl 4-(5-carbamoyl-4-((6-cyclopropylpyridin-3-yl)amino)pyrimidin-2-yl)-3-methylpiperazine-1-carboxylate (1.0 eq) at 0° C. and then stirring at RT for 16. Desired compound is an off white solid (25 mg, 65.7% yield). LCMS: m/z=354.2 (M+1), tRet: 1.79 and 1.80 min. HPLC: tRet: 5.11 min. ¹H NMR (400 MHz, DMSO-d6) δ 11.46 (s, 1H), 8.62 (s, 1H), 8.58 (d, J=2.4 Hz, 1H), 7.93 (dd, J₁=8.4 Hz, J₂=2.4 Hz, 2H), 7.30 (bs, 1H), 7.24 (d, J=8.4 Hz, 1H), 4.69 (bs, 1H), 4.31 (bs, 1H), 3.03-2.94 (m, 2H), 2.81-2.76 (m, 2H), 2.07-2.74 (m, 1H), 1.23-1.14 (m, 4H), 0.91-0.85 (m, 4H).

### Example 30

### Example 30A

### 4-((6-cyclopropylpyridin-3-yl)amino)-2-(2-methylpiperazin-1-yl)pyrimidine-5-carboxamide Hydrochloride

To a stirred solution of tert-butyl 4-(5-carbamoyl-4-((6-cyclopropylpyridin-3-yl)amino)pyrimidin-2-yl)-3-methylpiperazine-1-carboxylate (250 mg, 0.551 mmol, 1.0 eq.) in DCM (10 ml) is added 4M HCl in 1,4-dioxane (5 ml) at 0° C. The reaction mixture is stirred at RT for 2 h. Completion of the reaction is monitored by TLC. The reaction mixture is evaporated completely by rotary evaporator, and washed with diethyl ether (10 ml×2), dried under reduced pressure to afford the desired compound as an off white solid (200 mg, 93.45% yield). LCMS: m/z=354.10 (M+1)⁺, tRet: 1.05 min.

### Example 30B

### 2-(4-acetyl-2-methylpiperazin-1-yl)-4-((6-cyclopropylpyridin-3-yl)amino)pyrimidine-5-carboxamide (Table 1, Compound No. 11)

Synthesis is performed similar to Amide Formation Reaction and Examples above using (racemic) 4-((6-cyclopropylpyridin-3-yl)amino)-2-(2-methylpiperazin-1-yl)pyrimidine-5-carboxamide hydrochloride (1.0 eq), DCM, TEA (6 eq.), and acetyl chloride (1.3 eq) and stirring at 0° C. initially and then at RT for 2 h. Desired compound is a yellow solid (75 mg, 49% yield). LCMS: m/z=396.2 (M+1), tRet 1.88 and 1.89 min. ¹H NMR (400 MHz, DMSO-d₆) δ 11.31 (s, 1H), 8.63 (s, 1H), 8.55 (d, J=2.4 Hz, 1H), 7.90 (dd, J₁=8.4 Hz, J₂=2.48 Hz, 1H), 7.36 (bs, 2H), 7.19 (d, J i=8.4 Hz, 1H), 4.80-4.77 (m, 1H), 4.36 (dd, J₁=10.4 Hz, J₂=3.2 Hz, 1H), 4.30-3.50 (m, 2H), 3.45-3.10 (m, 2H), 2.45-2.44 (m, 1H), 2.09-2.03 (m, 4H), 1.12 (bs, 3H), 0.91-0.86 (m, 4H).

### Example 31

### Example 31A

### Tert-Butyl 4-(4-((6-cyclopropylpyridin-3-yl)amino)-5-(methylcarbamoyl)pyrimidin-2-yl)-3-methylpiperazine-1-carboxylate (Table 1, Compound No. 15)

Synthesis is performed similar to Second Heteroaryl Amination Reaction and Examples above using Intermediate 5 (1 eq), BuOH, tert-butyl 3-methylpiperazine-1-carboxylate (4.0 eq), and DIPEA (10.0 eq) and stirring for 1.5 h at 150° C. in microwave irradiation. Desired compound is a white solid (0.270 g, 50% yield). LCMS (Method A): m/z=468.3 (M+H)⁺. ¹H NMR (400 MHz, DMSO-d₆) δ=11.37 (s, 1H), 8.57 (s, 1H), 8.53 (d, J=2.0 Hz, 1H), 8.44-8.43 (m, 1H), 7.99 (dd, J₁=8.4 Hz, J₂=2.4 Hz, 1H), 7.27 (d, J=8.8 Hz, 1H), 4.78 (brs, 1H), 4.35 (brs, 1H), 3.95-3.79 (m, 2H), 3.14-3.11 (m, 3H), 2.76 (d, J=4.0 Hz, 3H), 2.08-2.04 (m, 1H), 1.43 (s, 9H), 1.11-1.10 (m, 3H), 0.93-0.84 (m, 4H).

### Example 31B

### 4-((6-cyclopropylpyridin-3-yl)amino)-N-methyl-2-(2-methylpiperazin-1-yl)pyrimidine-5-carboxamide Hydrochloride (Table 1, Compound No. 14)

Synthesis is performed similar to N-t-Boc Deprotection and Examples above using acetyl chloride (7.0 eq), MeOH, and racemic tert-butyl 4-(4-((6-cyclopropylpyridin-3-yl)amino)-5-(methylcarbamoyl)pyrimidin-2-yl)-3-methylpiperazine-1-carboxylate (1.0 eq) initially at 0° C. and then stirring at 30° C. for 16 h. Desired compound is an off white solid (0.050 g, 50% yield). LCMS (Method B): m/z=468.10 (M+H)⁺. ¹H NMR (400 MHz, DMSO-d₆) δ=11.92 (s, 1H), 9.80 (brs, 1H), 9.34 (brs, 1H), 9.33 (s, 1H), 8.89 (brs, 1H), 8.82 (s, 1H), 8.38-8.36 (m, 1H), 7.68 (d, J=9.2 Hz, 1H), 5.01 (brs, 2H), 4.65 (brs, 1H), 3.36-3.18 (m, 4H), 3.00-3.98 (m, 1H), 2.79 (d, J=4.0 Hz, 3H), 2.46-2.39 (m, 1H), 1.43-1.23 (m, 7H).

### Example 31C

### 2-(4-acetyl-2-methylpiperazin-1-yl)-4-((6-cyclopropylpyridin-3-yl)amino)-N-methylpyrimidine-5-carboxamide (Table 1, Compound No. 70)

Synthesis is performed similar to Amide Formation Reaction and Examples above using racemic 4-((6-cyclopropylpyridin-3-yl)amino)-N-methyl-2-(2-methylpiperazin-1-yl)pyrimidine-5-carboxamide hydrochloride (1.0 eq), DCM, triethylamine (5.0 eq), acetyl chloride (1.3 eq) at 0° C. initially and then stirring at 25-30° C. for 4 h. Desired compound is an off white solid (40 mg, 39% yield). LCMS (Method C): m/z=410.2 (M+H)⁺. ¹H NMR (400 MHz, DMSO-d₆) δ=11.20 (s, 1H), 8.57-8.55 (m, 2H), 8.18 (brs, 1H), 7.91 (d, J=8.4 Hz, 1H), 7.19 (d, J=8.4 Hz, 1H), 4.78 (brs, 1H), 4.37-4.34 (m, 1H), 3.29-3.25 (m, 2H), 2.78 (s, 3H), 2.07-2.03 (m, 4H), 1.19-1.12 (m, 6H), 0.91-0.86 (m, 4H).

### Example 32

### Example 32A

### Tert-Butyl 4-(5-carbamoyl-4-((2-methoxyphenyl)amino)pyrimidin-2-yl)-3-methylpiperazine-1-carboxylate (Table 1, Compound No. 43)

Synthesis is performed similar to Second Heteroaryl Amination Reaction and Examples above using Intermediate 3 (1.0 eq), 1-butanol, tert-butyl 3-methylpiperazine-1-carboxylate (1.5 eq), and DIPEA (3.0 eq) and stirring at 100° C. for 16 h in a sealed tube. Desired compound is a white solid. Yield: 0.09 g (39%). ¹H-NMR (400 MHz, DMSO-d₆): δ=11.65 (s, 1H), 8.62 (s, 1H), 8.38 (dd, 1H), 7.88 (brs, 1H), 7.2 (brs, 1H), 7.0 (m, 3H) 4.8 (brs, 1H), 4.4 (brs, 1H), 3.97-3.82 (m, 5H), 3.1-2.9 (m, 3H), 1.39 (s, 9H), 1.13 (d, 3H); LC-MS (Method A): [M+H]⁺=443.3; [M−H]⁺=441.2; tRet=2.530 min; HPLC (Method B): tRet=7.240 min.

### Example 32B

### 4-((2-methoxyphenyl)amino)-2-(2-methylpiperazin-1-yl)pyrimidine-5-carboxamide hydrochloride (Table 1, Compound No. 42)

Synthesis is performed similar to N-t-Boc Deprotection and Examples above using methanol, acetyl chloride (10.0 eq), and tert-butyl 4-(5-carbamoyl-4-((2-methoxyphenyl)amino)pyrimidin-2-yl)-3-methylpiperazine-1-carboxylate (racemic) (1.0 eq) initially at at 0° C. and then stirring for 4 h at RT. Desired compound is an off-white solid. Yield: 0.05 g (HCl salt). ¹H-NMR (400 MHz, DMSO-d₆): δ=12.00 (s, 1H), 9.7 (d, 1H), 9.4 (d, 1H), 8.71 (s, 1H), 8.27 (m, 2H), 7.54 (brs, 1H) 7.1 (d, 2H), 7.0 (m, 3H), 4.98 (m, 2H), 4.6 (m, 2H), 3.8 (s, 3H), 3.4-3.1 (m, 4H), 3.0 (m, 1H), 1.38 (d, 3H); LC-MS (Method A): Parent Mol. [M+H]⁺=343.2; [M−H]⁺=341.2; tRet=2.344 min. HPLC (Method B): tRet=5.191 min.

### Example 32C

### 2-(4-acetyl-2-methylpiperazin-1-yl)-4-((2-methoxyphenyl)amino)pyrimidine-5-carboxamide (Table 1, Compound No. 59)

Synthesis is performed similar to Amide Formation Reaction and Examples above using 4-((2-methoxyphenyl)amino)-2-(2-methylpiperazin-1-yl)pyrimidine-5-carboxamide hydrochloride (racemic) (1.0 eq), DCM, pyridine (5.0 eq), and acetyl chloride (2.0 eq) and stirring at RT for 16 h. Desired compound is an off-white solid. Yield: 0.120 g (79%). ¹H-NMR (400 MHz, DMSO-d₆): δ=11.47 (s, 1H) 8.6 (s, 1H), 8.34 (dd, J=6.4, 1.2 Hz, 1H), 7.25 (brs, 2H), 4.83 (m, 1H), 4.4 (m, 1H), 4.2 (brs, 1H), 3.6 (brm, 4H), 3.04 (s, 3H), 2.071 (s, 3H), 1.1 (s, 3H); LC-MS (Method A): [M+H]=385.2; [M−H]⁺=383.2; tRet=2.052 min; HPLC (Method D): tRet=5.757 min at 210 nm, tRet=5.763 min at 255 nm.

### Example 33

### Example 33A

### Tert-Butyl 4-(4-((2-methoxyphenyl)amino)-5-(methylcarbamoyl)pyrimidin-2-yl)-3-methylpiperazine-1-carboxylate (Table 1, Compound No. 32)

Synthesis is performed similar to Second Heteroaryl Amination Reaction and Examples above using 2-chloro-4-((2-methoxyphenyl)amino)-N-methylpyrimidine-5-carboxamide (1.0 eq), n-butanol, DIPEA (3.0 eq), and tert-butyl 3-methylpiperazine-1-carboxylate (1.5 eq) and heating to 140° C. for 16 h. Desired compound is an off white solid yield: 0.200 g, 64.08%. LC-MS (Method B): (2M+H)⁺: 913.45; (M−H)⁺: 455.15; tRet: 1.48 min, HPLC (Method B): tRet: 7.46 min.

### Example 33B

### 4-((2-methoxyphenyl)amino)-N-methyl-2-(2-methylpiperazin-1-yl)pyrimidine-5-carboxamide Hydrochloride (Table 1, Compound No. 31)

Synthesis is performed similar to N-t-Boc Deprotection and Examples above using tert-butyl 4-(4-((2-methoxyphenyl)amino)-5-(methylcarbamoyl)pyrimidin-2-yl)-3-methylpiperazine-1-carboxylate (1.0 eq), methanol, 4M HCl in 1,4-dioxane and stirring at RT for 3 h. Desired compound is an off white solid yield: 0.040 g. ¹H NMR (400 MHz, DMSO-d₆) δ=11.63 (brs, 1H), 9.37 (brs, 1H), 9.00 (brs, 1H), 8.62 (m, 1H), 8.53 (brs, 1H), 8.31 (d, 1H, J=8.0 Hz), 7.08-6.95 (m, 3H), 4.99 (brs, 1H), 4.65 (d, 1H, 13.2 Hz), 3.87 (s, 1H), 3.36-2.98 (m, 7H), 2.77 (d, 3H, J=4.4 Hz), 1.33 (d, 3H, J=6.8 Hz). LC-MS (Method B): 356.20 (2M+H)⁺: 713.35; (M−H)⁺: 355.15; tRet: 1.16 min; HPLC (Method B): tRet: 5.29 min.

### Example 33C

### 2-(4-acetyl-2-methylpiperazin-1-yl)-4-((2-methoxyphenyl)amino)-N-methylpyrimidine-5-carboxamide (Table 1, Compound No. 23)

Synthesis is performed similar to Amide Formation Reaction and Examples above using (racemic) 4-((2-methoxyphenyl)amino)-N-methyl-2-(2-methylpiperazin-1-yl)pyrimidine-5-carboxamide hydrochloride (1.0 eq), DCM, pyridine (6.0 eq), and acetyl chloride (3.0 eq) initially at 0° C. and then stirring for 4 h at RT. Desired compound is a white solid. Yield: 0.040 g, 49.48%. ¹H NMR (400 MHz) VT at 90° C. DMSO-d₆ 6=11.31 (s, 1H), 8.54 (s, 1H), 8.34 (d, 1H, J=8.0 Hz), 8.06 (brs, 1H), 7.04-6.92 (m, 3H), 4.82 (brs, 1H), 4.41 (d, 1H, J=13.2 Hz), 4.20 (br, 1H), 3.90 (s, 3H), 3.29 (brs, 2H), 2.78 (d, 3H, J=4.4 Hz), 2.03 (s, 3H), 1.14 (brs, 3H). LC-MS (Method A): (M+H)⁺: 399.2; (M−H)⁺: 397.1; tRet: 2.09 min. HPLC (Method B): 210 nm tRet: 5.94 min.

### Example 34

### Example 34A

### (Racemic) Tert-Butyl 4-(5-carbamoyl-4-((2-(methylsulfonyl)phenyl)amino)pyrimidin-2-yl)-3-methylpiperazine-1-carboxylate (Table 1, Compound No. 55)

Synthesis is performed similar to Second Heteroaryl Amination Reaction and Examples above and using Intermediate 8 (1.0 eq), nBuOH, (racemic) tert-butyl 3-methylpiperazine-1-carboxylate (1.5 eq), and DIPEA (15 eq) and stirring for 16 h at 150° C. Desired compound is an off-white solid (0.51 g, 75% yield). ¹H NMR (400 MHz, DMSO-d₆): δ=11.45 (s, 1H), 8.67 (s, 1H), 8.17 (d, J=8.4 Hz, 1H), 7.90 (d, J=8.0 Hz, 1H), 7.68 (t, J=7.6 Hz, 1H), 7.33-7.30 (m, 3H), 4.71-4.65 (m, 1H), 4.29 (d, J=13.6 Hz, 1H), 3.87 (d, J=13.2 Hz, 1H), 3.74 (d, J=13.6 Hz, 1H), 3.17-3.06 (m, 5H), 2.94-2.90 (m, 1H), 1.41 (s, 9H), 1.09 (d, J=6.8 Hz, 3H). LCMS (Method-B): m/z=491.15 (M+H)⁺, tRet: 1.39 min, HPLC (Method-A): tRet: 6.89 min.

### Example 34B

### (Racemic) 2-(2-methylpiperazin-1-yl)-4-((2-(methylsulfonyl)phenyl)amino)pyrimidine-5-carboxamide hydrochloride (Table 1, Compound No. 63)

Synthesis is performed similar to N-t-Boc Deprotection and Examples above using methanol, acetyl chloride (30 eq), and (racemic) tert-butyl 4-(5-carbamoyl-4-((2-(methylsulfonyl)phenyl)amino)pyrimidin-2-yl)-3-methylpiperazine-1-carboxylate (1.0 eq) initially at 0° C. and then stirring for 16 h at RT. Desired compound is an off-white solid (0.15 g, 78% yield). ¹H NMR (400 MHz, DMSO-d₆): δ=11.69 (s, 1H), 9.61-9.58 (m, 1H), 9.24-9.21 (m, 1H), 8.76 (s, 1H), 8.16 (d, J=8.4 Hz, 1H), 8.06 (bs, 1H), 7.91 (dd, J₁=1.2 Hz, J₂=7.6 Hz, 1H), 7.671 (t, J=7.2 Hz, 1H), 7.38-7.34 (m, 2H), 4.89 (bs, 1H), 4.54 (bs, 1H), 3.28-3.07 (m, 7H), 2.95-2.90 (m, 1H), 1.30 (d, J=7.2 Hz, 3H). LCMS (Method-B): m/z=391.10 (M+H)⁺ tRet: 1.10 min. HPLC (Method-E): tRet: 5.33 min.

### Example 34C

### (Racemic) 2-(4-acetyl-2-methylpiperazin-1-yl)-4-((2-(methylsulfonyl)phenyl)amino)pyrimidine-5-carboxamide (Table 1, Compound No. 62)

Synthesis is performed similar to Amide Formation Reaction and Examples above using (racemic) 2-(2-methylpiperazin-1-yl)-4-((2-(methylsulfonyl)phenyl)amino)pyrimidine-5-carboxamide hydrochloride (1.0 eq), DCM, pyridine (5.0 eq), and acetyl chloride (2.0 eq) and stirring for 3 h at RT. Desired compound is an off-white solid (0.12 g, 59% yield). ¹H NMR (400 MHz, DMSO-d₆): δ=11.44 (s, 1H), 8.67 (s, 1H), 8.17 (d, J=8.4 Hz, 1H), 7.90 (dd, J₁=1.6 Hz, J₂=8.0 Hz, 1H), 7.67 (t, J=7.2 Hz, 1H), 7.40-7.30 (m, 3H), 4.72-4.69 (m, 1H), 4.32-4.29 (m, 1H), 4.24-3.56 (m, 2H), 3.45-3.15 (m, 2H), 3.14 (s, 3H), 2.92-2.75 (m, 1H), 2.05 (s, 3H), 1.09 (d, J=4.8 Hz, 3H). LCMS (Method-A): m/z=433.2 (M+H)⁺ tRet: 2.00 min. HPLC (Method-A): tRet: 5.65 min.

### Example 35

### Example 35A

### Tert-Butyl 3-methyl-4-(5-(methylcarbamoyl)-4-((2-(methylsulfonyl)phenyl)amino)pyrimidin-2-yl)piperazine-1-carboxylate (Table 1, Compound No. 18)

Synthesis is performed similar to Second Heteroaryl Amination Reaction and Examples above and using Intermediate 9 (1.0 eq), n-BuOH, tert-butyl 3-methylpiperazine-1-carboxylate (5.0 eq), and DIPEA (5.0 eq) and stirring for 1.5 h at 150° C. in microwave irradiation. Desired compound is an off white solid (0.180 g, 61% yield). ¹H NMR (400 MHz, DMSO-d₆): δ ppm=11.46 (s, 1H), 8.63 (s, 1H), 8.39 (d, J=4.8 Hz, 1H), 8.16 (d, J=8.4 Hz, 1H), 7.89 (dd, J=1.2 Hz, J=8.0 Hz, 1H), 7.71 (t, J=7.2 Hz, 1H), 7.33 (t, J=7.6 Hz, 1H), 4.76 (bm, 1H), 4.33 (bm, 1H), 3.91 (bm, 1H), 3.76 (d, J=12.4 Hz, 1H), 3.19 (s, 3H), 3.09-2.79 (m, 3H), 2.76 (bs, 3H), 1.53 (s, 9H), 1.07 (d, J=6.8 Hz, 3H). LC-MS (Method B): M+1: 505.10, M−1: 503.05, (M-tertbutyl)+1: 449.05, tRet: 1.409 min and HPLC (Method B): tRet: 7.249 min.

### Example 35B

### N-methyl-2-(2-methylpiperazin-1-yl)-4-((2-(methylsulfonyl)phenyl)amino)pyrimidine-5-carboxamide Hydrochloride (Table 1, Compound No. 69)

Synthesis is performed similar to N-t-Boc Deprotection and Examples above using racemic tert-butyl 3-methyl-4-(5-(methylcarbamoyl)-4-((2-(methylsulfonyl)phenyl)amino)pyrimidin-2-yl)piperazine-1-carboxylate (1.0 eq), DCM, and 4M HCl in 1,4-dioxane (10.0 eq) initially at 0° C. and then stirring for 2 h at rt. Desired compound is an off white solid (0.150 g, 96% yield). ¹H NMR (400 MHz, DMSO-d₆): δ ppm=11.57 (s, 1H), 9.52 (d, J=10 Hz, 1H), 9.15 (d, J=10 Hz, 1H), 8.72 (s, 1H), 8.61 (d, J=4.0 Hz, 1H), 8.10 (d, J=8.4 Hz, 1H), 7.91 (d, J=6.8 Hz, 1H), 7.71 (t, J=7.2 Hz, 1H), 7.37 (t, J=7.6 Hz, 1H), 3.38-3.08 (m, 8H), 2.96-2.91 (m, 1H), 2.77 (d, J=4.4 Hz, 3H), 1.28 (d, J=6.8 Hz, 3H). LC-MS (Method B): M+1: 405.10, M−1: 403.05, 2M+1: 809.25, tRet: 1.117 min and HPLC (Method B): tRet: 5.407 min.

### Example 35C

### 2-(4-acetyl-2-methylpiperazin-1-yl)-N-methyl-4-((2-(methylsulfonyl)phenyl)amino)pyrimidine-5-carboxamide (Table 1, Compound No. 76)

Synthesis is performed similar to Amide Formation Reaction and Examples above using racemic N-methyl-2-(2-methylpiperazin-1-yl)-4-((2-(methylsulfonyl)phenyl)amino)pyrimidine-5-carboxamide hydrochloride (1.0 eq), DCM, triethylamine (5.0 eq), and acetyl chloride (1.5 eq) initially at 0° C. and then stirring for 2 h at rt. After work-up, product is purified by preparative HPLC. After preparative HPLC compound is lyophilized for 72 h. Desired product is an off white solid (0.030 g, 30%). LC-MS (Method B): M+1: 447.15, M−1: 445.05, 2M+1: 893.25, tRet: 1.209 min and HPLC (Method G): tRet: 6.191 min. ¹H NMR (400 MHz, DMSO-d₆): δ ppm=11.46 (s, 1H), 8.64 (s, 1H), 8.40 (d, J=4.0 Hz, 1H), 8.15 (d, J=8.4 Hz, 1H), 7.90 (dd, J=8.0 Hz, J=1.2 Hz, 1H), 7.70 (t, J=7.6 Hz, 1H), 7.34 (t, J=8.0 Hz, 1H), 4.89-4.56 (bs, 1H), 4.45-4.09 (m, 2H), 3.84-3.68 (m, 1H), 3.29-3.03 (m, 5H), 2.89-2.67 (m, 4H), 2.06 (d, J=7.6 Hz, 3H), 1.12-1.09 (m, 3H).

### Example 36

Example 36A tert-butyl 4-(S-carbamoyl-4-((3-(5-fluoropyrimidin-2-yl)-2-methoxyphenyl)amino)pyrimidin-2-yl)-1,4-diazepane-1-carboxylate (Table 1, Compound No. 71)

To a stirred solution of 2-chloro-4-((3-(5-fluoropyrimidin-2-yl)-2-methoxyphenyl)amino)pyrimidine-5-carboxamide (250 mg, 0.66 mmol, 1.0 eq) in BuOH (10 mL), tert-butyl 1,4-diazepane-1-carboxylate (401 mg, 2.00 mmol, 3.0 eq) and DIPEA (0.58 mL, 3.30 mmol, 5.0 eq) are added. The reaction mixture is stirred for 1.5 h at 150° C. in microwave irradiation. The reaction mixture turns into a yellow color. Completion of the reaction is monitored by TLC. The reaction mixture is concentrated under reduced pressure to afford the crude compound. The crude compound is purified by MPLC using MeOH/CH₂Cl₂ (gradient 3-5% CH₂Cl₂) to afford the desired compound as an off white solid (250 mg, 69% yield). LCMS (Method B): m/z=539.35 (M+H)⁺ tRet: 2.36 min; HPLC (Method D): tRet: 6.723 min. ¹H NMR (400 MHz, DMSO-d₆) δ=11.78 (s, 1H), 8.94 (s, 2H), 8.63-8.60 (m, 2H), 7.35-7.29 (bs, 3H), 7.19 (t, J=8.0 Hz, 1H), 3.89 (t, J=8.0 Hz, 2H), 3.79-3.78 (m, 2H), 3.56 (m, 2H), 3.33 (t, J=5.2 Hz, 2H), 1.84 (bs, 2H), 1.29 (s, 9H).

### Example 36B

### 2-(1,4-diazepan-1-yl)-4-((3-(5-fluoropyrimidin-2-yl)-2-methoxyphenyl)amino)pyrimidine-5-carboxamide Hydrochloride (Table 1, Compound No. 73)

To a stirred solution of tert-butyl 4-(5-carbamoyl-4-((3-(5-fluoropyrimidin-2-yl)-2-methoxyphenyl)amino)pyrimidin-2-yl)-1,4-diazepane-1-carboxylate (200 mg, 0.371 mmol, 1.0 eq) in DCM (10 mL) at RT, is added HCl in dioxane (0.92 mL, 3.710 mmol, 10.0 eq). Then the reaction mixture is stirred for 2 h at RT. Completion of the reaction is monitored by TLC. The reaction mixture is concentrated under reduced pressure to afford the crude compound and triturated with diethyl ether and dried under high vacuum to afford the desired compound as an off white solid (150 mg, 85% yield). LCMS (Method B): m/z=439.15 (M+H)⁺ tRet: 1.13 min, HPLC: tRet: 5.093 min. ¹H NMR (400 MHz, DMSO-d₆) δ=11.93 (s, 1H), 9.25 (s, 2H), 8.95 (s, 2H), 8.69 (s, 1H), 8.50 (d, J=8.0 Hz, 1H), 7.40 (d, J=7.6 Hz, 1H), 7.24 (t, J=8.0 Hz, 1H), 4.26-4.08 (m, 4H), 3.94 (d, J=5.2 Hz, 2H), 3.31 (bs, 2H), 3.19 (bs, 2H), 2.13 (bs, 2H).

### Example 36C

### 2-(4-acetyl-1,4-diazepan-1-yl)-4-((3-(5-fluoropyrimidin-2-yl)-2-methoxyphenyl)amino)pyrimidine-5-carboxamide (Table 1, Compound No. 81)

To a stirred solution of 2-(1,4-diazepan-1-yl)-4-((3-(5-fluoropyrimidin-2-yl)-2-methoxyphenyl)amino)pyrimidine-5-carboxamide hydrochloride (100 mg, 0.2 mmol, 1.0 eq) in DCM (10 mL) at 0° C. is added triethylamine (0.17 mL, 1.1 mmol, 5.0 eq), followed by acetyl chloride (0.03 mL, 0.454 mmol, 2.0 eq). The reaction mixture is stirred for 2 h at RT. Completion of the reaction is monitored by TLC. The reaction mixture is concentrated under reduced pressure to afford the crude compound. The crude compound is purified by preparative HPLC. The fractions are lyophilized to afford the desired compound as an off white solid (25 mg, 25% yield). LCMS (Method A): m/z=481.2 (M+H)⁺, tRet: 2.023 min HPLC (Method B): tRet: 5.677 min. ¹H NMR (400 MHz, DMSO-d₆) δ=11.77 (s, 1H), 8.94 (s, 1H), 8.63 (s, 1H), 8.58 (bs, 1H), 7.36-7.32 (m, 3H), 7.20 (t, J=7.6 Hz, 1H), 3.95-3.81 (m, 4H), 3.66-3.63 (m, 5H), 3.45 (bs, 2H), 1.98-1.81 (m, 5H).

### Example 37

### Example 37A

### Tert-Butyl 4-(4-((3-(5-fluoropyrimidin-2-yl)-2-methoxyphenyl)amino)-5-(methylcarbamoyl)pyrimidin-2-yl)-1,4-diazepane-1-carboxylate (Table 1, Compound No. 72)

Synthesis is performed similar to Second Heteroaryl Amination Reaction and Examples above and using Intermediate 2 (1.0 eq), 1,4-dioxane, TEA (3.0 eq), and tert-butyl 1,4-diazepane-1-carboxylate (1.5 eq) and stirring at 100° C. for 16 h in a sealed tube. Desired compound is an off white solid (0.50 g, 70%, yield). LCMS (Method B): m/z=553.20 (M+H), 551.15 (M−H) tRet: 1.41; HPLC (Method D): tRet: 6.840. ¹H NMR (400 MHz, DMSO-d₆): 11.65 (s, 1H), 8.94 (s, 2H), 8.58-8.55 (m, 2H), 8.16 (brs, 1H), 7.37 (d, J=7.6 Hz, 1H), 7.20 (t, J=8.0 Hz, 1H), 3.88 (t, J=6.0 Hz, 2H), 3.79 (t, J=6.0 Hz, 2H), 3.69 (s, 3H), 3.56 (brs, 2H), 3.34 (t, J=6.0 Hz, 2H), 2.78 (d, J=3.2 Hz, 3H), 1.83 (brs, 2H), 1.25 (s, 9H).

### Example 37B

### 2-(1,4-diazepan-1-yl)-4-((3-(5-fluoropyrimidin-2-yl)-2-methoxyphenyl)amino)-N-methylpyrimidine-5-carboxamide Hydrochloride (Table 1, Compound No. 74)

Synthesis is performed similar to N-t-Boc Deprotection and Examples above using tert-butyl 4-(4-((3-(5-fluoropyrimidin-2-yl)-2-methoxyphenyl)amino)-5-(methylcarbamoyl)pyrimidin-2-yl)-1,4-diazepane-1-carboxylate (1.0 eq), DCM, and HCl in dioxane and stirring for 2 h at RT. Desired compound is an off white solid (300 mg, 92% yield). LCMS (Method A): m/z=453.2 (M+H), tRet: 1.87, 451.2 (M−H); HPLC (Method D): tRet: 5.158. ¹H NMR (400 MHz, DMSO-d₆): 11.75 (s, 1H), 9.20 (brs, 2H), 8.95 (s, 2H), 8.63 (s, 1H), 8.46 (d, J=7.6 Hz, 1H), 8.39 (brs, 1H), 7.39 (d, J=6.4 Hz, 1H), 7.23 (t, J=7.6 Hz, 1H), 4.07 (t, J=5.2 Hz, 2H), 3.92 (t, J=6.0 Hz, 2H), 3.70 (s, 3H), 3.30 (brs, 2H), 3.19 (brs, 2H), 2.77 (s, 3H), 2.15-2.10 (m, 2H).

### Example 37C

### 2-(4-acetyl-1,4-diazepan-1-yl)-4-((3-(5-fluoropyrimidin-2-yl)-2-methoxyphenyl)amino)-N-methylpyrimidine-5-carboxamide (Table 1, Compound No. 77)

Synthesis is performed similar to Amide Formation Reaction and Examples above using 2-(1,4-diazepan-1-yl)-4-((3-(5-fluoropyrimidin-2-yl)-2-methoxyphenyl)amino)-N-methylpyrimidine-5-carboxamide hydrochloride (1.0 eq), DCM, TEA (5.0 eq), and acetyl chloride (1.3 eq) and stirring for 2 h at RT. After preparative HPLC purification the compound is lyophilized for 48 h to afford the desired compound as an off white solid (30 mg, 43% yield). LCMS (Method B): tRet: 1.225, m/z=495.25 (M+H), 493.35 (M−H); HPLC (Method F): tRet: 6.455. ¹H NMR (400 MHz, DMSO-d₆): 11.58 (s, 1H), 8.94 (s, 2H), 8.55 (s, 2H), 8.11 (brs, 1H), 7.34 (d, J=6.8 Hz, 1H), 7.20 (t, J=8.0 Hz, 1H), 3.94-3.79 (m, 4H), 3.69 (s, 3H), 3.64 (t, J=5.6 Hz, 2H), 3.45 (brs, 2H), 2.77 (d, J=4.4 Hz, 3H), 2.02-1.81 (m, 5H).

### Example 38

### Example 38A

### Tert-Butyl 4-(S-carbamoyl-4-((6-cyclopropylpyridin-3-yl)amino)pyrimidin-2-yl)-1,4-diazepane-1-carboxylate (Table 1, Compound No. 47)

Synthesis is performed similar to Second Heteroaryl Amination Reaction and Examples above and using Intermediate 4 (1.0 eq), tert-butyl 1,4-diazepane-1-carboxylate (5.0 eq), N,N diisopropylethylaamine, and n-butanol and stirring at 130° C. for 16 h in a sealed tube. Desired compound is an off-white solid. Yield: 140.0 mg, 36%. ¹H NMR (400 MHz) DMSOd₆ δ=11.31 (1H, s), 8.60 (s, 1H), 8.57-8.56 (d, J=4.0 Hz, 1H), 7.93-7.90 (m, 1H), 7.32 (s, 2H), 7.19-7.17 (d, J=8.0 Hz, 1H), 3.84-3.73 (m, 4H), 3.52-3.51 (m, 2H), 3.32-3.29 (m, 2H), 2.06-203 (m, 1H), 1.77 (m, 2H), 1.28 (s, 9H), 0.90-0.85 (m, 4H). HPLC: tRet=5.82 (Method-B) LC-MS: (M−1): 452.3, tRet=1.208, (M+1): 454.3, tRet=1.207 (Method-A).

### Example 38B

### 4-((6-cyclopropylpyridin-3-yl)amino)-2-(1,4-diazepan-1-yl)pyrimidine-5-carboxamide Hydrochloride (Table 1, Compound No. 46)

Synthesis is performed similar to N-t-Boc Deprotection and Examples above using acetyl chloride (20.0 eq), methanol, tert-butyl 4-(5-carbamoyl-4-((6-cyclopropylpyridin-3-yl)amino)pyrimidin-2-yl)-1,4-diazepane-1-carboxylate (1.0 eq) initially at 0° C. and then stirring at RT for 6 h. Desired compound is an off-white solid compound. Yield: 80.0 mg, 93%. DMSOd₆ (400 mHz) V.T. NMR at 90° C. DMSOd₆ (400 MHz) DMSOd₆+D₂O Exchange (400 MHz) δ=11.71 (1H, s), 9.15-9.13 (d, 2H), 8.97 (s, 1H), 8.717 (s, 1H), 8.137-8.115 (m, 1H), 7.45-7.43 (m, 2H), 4.05-4.04 (m, 2H), 3.90-3.87 (m, 2H), 3.186 (m, 6H), 2.31-2.24 (m, 1H), 2.14-2.10 (m, 2H), 1.15-1.04 (m, 4H). HPLC (Method-D): tRet=5.125, LC-MS (Method C): Parent M.Wt: 353.20, HCl salt M.Wt: 389.17 [M+1]+354.2, tRet=1.756 [M−1]⁺: 352.3, tRet=1.756.

### Example 39

### 2-(4-acetyl-1,4-diazepan-1-yl)-4-((6-cyclopropylpyridin-3-yl)amino)pyrimidine-5-carboxamide (Table 1, Compound No. 64)

Synthesis is performed similar to Amide Formation Reaction and Examples above using 4-((6-cyclopropylpyridin-3-yl)amino)-2-(1,4-diazepan-1-yl)pyrimidine-5-carboxamide (1.0 eq), DCM, pyridine (4.0 eq), and acetyl chloride (1.5 eq) initially at 0° C. and then stirring at RT for 1 h. Desired compound is a white solid. Yield: 30.0 mg, 16%. ¹H NMR DMSOd₆ (400 MHz) V.T. NMR at 90° C. DMSOd₆ (400 MHz) δ=11.29 (1H, s), 8.60 (s, 1H), 8.558 (d, J=2.0 Hz, 1H), 7.91 (d, J=6.8 Hz, 1H), 7.3 (m, 2H), 7.20 (d, J=8.4 Hz, 1H), 3.88 (m, 4H), 3.77 (m, 2H), 3.61 (m, 2H), 2.07 (m, 1H), 1.96 (m, 5H), 0.88 (m, 4H). HPLC: tRet=5.05, tRet=5.06, (Method A); LC-MS: Parent M.wt: 395.21 (M+1)⁺: 396.2, tRet=1.81, (M−1)⁺: 394.2, tRet=1.81. (Method A)

### Example 40

### Tert-Butyl 4-(4-((6-cyclopropylpyridin-3-yl)amino)-5-(methylcarbamoyl)pyrimidin-2-yl)-1,4-diazepane-1-carboxylate

Synthesis is performed similar to Second Heteroaryl Amination Reaction and Examples above and using Intermediate 5 (1.0 eq), BuOH, tert-butyl 1,4-diazepane-1-carboxylate (4.0 eq), and DIPEA (10.0 eq) and stirring for 1.5 h at 150° C. in microwave irradiation. Desired compound is a white solid. ¹H NMR (400 MHz, DMSO-d₆) δ ppm: 6=11.99 (s, 1H), 8.57 (d, J=3.6 Hz, 1H), 8.53 (s, 1H), 8.15 (d, J=3.6 Hz, 1H), 7.93 (dd, J₁=8.93 Hz, J₂=8.84 Hz, 2H), 7.18 (d, J=8.4 Hz, 1H), 3.83-3.73 (m, 4H), 3.50 (t, J=6.0 Hz, 2H), 3.30 (t, 5.6 Hz, 2H), 2.77 (d, J=4.4 Hz, 3H), 2.60-2.02 (m, 1H), 1.77 (s, 2H), 1.28 (s, 9H), 0.91-0.85 (m, 4H). LC-MS (0.270 g, 50% yield). LCMS (Method B): m/z=M+1: 468.2, tRet: 1.27 min and HPLC (Method D): HPLC: tRet=7.48 min.

### 4-((6-cyclopropylpyridin-3-yl)amino)-2-(1,4-diazepan-1-yl)-N-methylpyrimidine-5-carboxamide Hydrochloride (Table 1, Compound No. 22)

Synthesis is performed similar to N-t-Boc Deprotection and Examples above using tert-butyl 4-(4-((6-cyclopropylpyridin-3-yl)amino)-5-(methylcarbamoyl)pyrimidin-2-yl)-1,4-diazepane-1-carboxylate (1.0 eq), DCM (5.0 mL), 4N HCl in dioxane (3.0 eq) and stirring for 3 h at 25-30° C. Desired compound is a white solid. ¹H NMR (400 MHz, DMSO-d₆): δ ppm=11.66 (s, 1H), 9.23 (s, 2H), 9.04 (s, 1H), 8.72 (s, 1H), 8.54 (brs, 1H), 8.18 (dd, J₁=8.8 Hz, J₂=8.4 Hz, 1H), 7.45 (d, J=8.8 Hz, 1H), 4.05 (d, J=4.8 Hz, 2H), 3.89 (t, J=6.0 Hz, 2H), 3.32 (brs, 2H), 3.17 (d, J=4.4 Hz, 2H), 2.80 (s, 3H), 2.31 (d, J=4.0 Hz, 1H), 2.14-2.10 (m, 2H), 1.18-1.08 (m, 4H). LC-MS (0.100 g, 85% yield). LCMS (Method B): m/z=M+1 & M−1: 368.1 & 366.15, tRet: 1.07 min; HPLC (Method D): tRet: 5.20 min.

### Example 41

### 2-(4-acetyl-1,4-diazepan-1-yl)-4-((6-cyclopropylpyridin-3-yl)amino)-N-methylpyrimidine-5-carboxamide (Table 1, Compound No. 21)

To a stirred solution of 4-((6-cyclopropylpyridin-3-yl)amino)-2-(1,4-diazepan-1-yl)-N-methylpyrimidine-5-carboxamide hydrochloride (0.075 g, 0.186 mmol, 1.0 eq) in 1,4 dioxane (5.0 mL) is added acetic anhydride (0.2 mL, 1.86 mmol, 10.0 eq) and DIPEA (0.12 mL, 0.744 mmol, 4.0 eq). The reaction mixture is stirred for 5 h at 80° C. Completion of the reaction is monitored by TLC. The reaction mixture is quenched with cold water solution, then extracted into ethyl acetate (10 ml×2). Ethyl acetate layer is washed with cold water and brine solution. Organic layer is dried over sodium sulfate and concentrated under reduced pressure to afford the crude compound. The crude compound is triturated with pentane to afford the desired compound as a white solid (0.040 g, 52% yield). NMR in DMSO-d6: δ=11.17 (s, 1H), 8.55 (d, J=2.0 Hz, 1H), 8.53 (s, 1H), 8.14 (brs, 1H), 7.92 (d, J=7.6 Hz, 1H), 7.19 (d, J=8.4 Hz, 1H), 3.88-3.75 (m, 4H), 3.6 (d, J=6.0 Hz, 2H), 3.42 (d, J=3.6 Hz, 2H), 2.77 (d, J=4.4 Hz, 3H), 2.06-1.87 (m, 6H), 0.90-0.85 (m, 4H); LC MS (Method B): m/z=M+1 & M−1: 410.2 & 408.1, tRet: 1.14 min and HPLC (Method D): tRet: 5.22 min.

### Example 42

### Example 42A

### Tert-Butyl 4-(5-carbamoyl-4-((2-methoxyphenyl)amino)pyrimidin-2-yl)-1,4-diazepane-1-carboxylate (Table 1, Compound No. 41)

Synthesis is performed similar to Second Heteroaryl Amination Reaction and Examples above and using Intermediate 3 (1.0 eq), n-butanol, DIPEA (5.0 eq), and tert-butyl 1,4-diazepane-1-carboxylate (1.5 eq) and stirring at 140° C. for 16 h in a sealed tube. Desired compound is an off-white solid (300 mg, 75% yield). ¹H NMR (400 MHz, DMSO-d₆) δ=11.48 (s, 1H), 8.58 (s, 1H), 8.42-8.40 (dd, J₁=1.2 Hz, J₂=8.0 Hz, 1H), 7.20 (bs, 2H), 7.03-6.90 (m, 3H), 3.86-3.85 (m, 2H), 3.84 (s, 3H), 3.77-3.74 (m, 2H), 3.53 (t, J=8.0 Hz, 2H), 3.33-3.30 (m, 2H), 1.81 (bs, 2H), 1.29 (s, 9H). LCMS (Method B): m/z=443.15 (M+H), tRet: 2.62 min. HPLC (Method A): tRet: 7.58 min.

### Example 42B

### 2-(1,4-diazepan-1-yl)-4-((2-methoxyphenyl)amino)pyrimidine-5-carboxamide hydrochloride (Table 1, Compound No. 40)

Synthesis is performed similar to N-t-Boc Deprotection and Examples above using tert-butyl 4-(5-carbamoyl-4-((2-methoxyphenyl)amino)pyrimidin-2-yl)-1,4-diazepane-1-carboxylate (1.0 eq), MeOH, and acetyl chloride (15.0 eq) initially at 0° C. and then stirring at RT for 16 h. Desired compound is an off-white solid (40 mg, 85% yield). ¹H NMR (400 MHz, DMSO-d₆) δ=12.17 (bs, 1H), 9.36 (bs, 2H), 8.69 (s, 1H), 8.34-8.22 (m, 2H), 7.64 (bs, 1H), 7.12-7.01 (m, 3H), 4.11-4.02 (m, 4H), 3.88 (s, 3H), 3.33-3.10 (m, 5H), 2.12 (bs, 2H), ¹H NMR (400 MHz, DMSO-d₆, VT NMR): b=11.75 (s, 1H), 9.31 (bs, 2H), 8.65 (s, 1H), 8.27 (d, J=7.6 Hz, 1H), 7.9-7.1 (bs, 1H), 7.07-7.06 (m, 2H), 6.99-6.95 (m, 1H), 4.08-4.05 (m, 2H), 3.90 (t, J=6.0 Hz, 2H), 3.89 (s, 3H), 3.28-3.18 (m, 5H), 2.15-2.09 (m, 2H). ¹H NMR (400 MHz, DMSO-d₆, D₂0 exchange): b=9.02 (s, 2H), 8.62 (s, 1H), 8.36-8.23 (m, 1H), 7.12-7.03 (m, 3H), 4.02-3.94 (m, 4H), 3.88 (s, 3H), 3.33-3.24 (m, 5H), 2.12 (bs, 2H). LCMS (Method B): m/z=342.40 (M+H)⁺, tRet: 1.12 min. HPLC (Method B): tRet: 5.07 min.

### Example 42C

### 2-(4-acetyl-1,4-diazepan-1-yl)-4-((2-methoxyphenyl)amino)pyrimidine-5-carboxamide (Table 1, Compound No. 58)

Synthesis is performed similar to Amide Formation Reaction and Examples above using 2-(1,4-diazepan-1-yl)-4-((2-methoxyphenyl)amino)pyrimidine-5-carboxamide hydrochloride (1.0 eq), DCM, pyridine (6.0 eq), and acetyl chloride (2.0 eq) initially at 0° C. and then stirring at RT for 2 h. The collected fractions from preparative HPLC are lyophilized for 48 h to afford the desired compound as an off-white solid (20 mg, 21%, yield). ¹H NMR (400 MHz, DMSO-d₆) δ=11.46 (s, 1H), 8.57 (s, 1H), 8.38 (bs, 1H), 7.21 (bs, 2H), 7.03-6.93 (m, 3H), 3.92-3.78 (m, 7H), 3.63-3.61 (m, 2H), 3.43 (bs, 2H), 1.97-1.76 (m, 5H). LCMS (Method B): m/z=385.15 (M+H), tRet: 1.19 min, HPLC (Method B): tRet: 5.56 min.

### Example 43

### Example 43A

### Tert-Butyl 4-(4-((2-methoxyphenyl)amino)-5-(methylcarbamoyl)pyrimidin-2-yl)-1,4-diazepane-1-carboxylate (Table 1, Compound No. 30)

Synthesis is performed similar to Second Heteroaryl Amination Reaction and Examples above using 2-chloro-4-((2-methoxyphenyl)amino)-N-methylpyrimidine-5-carboxamide (1.0 eq), n-butanol, DIPEA (3.0 eq), and tert-butyl 1,4-diazepane-1-carboxylate (1.5 eq) and stirring at 140° C. for 16 h. Desired compound is an off white solid. Yield: 0.240 g, 76.90%. ¹H NMR (400 MHz) VT at 90° C. DMSO-d6: δ=11.33 (brs, 1H), 8.51 (s, 1H), 8.42 (dd, 1H, J=7.6, 8.0 Hz), 8.02 (brs, 1H), 7.03-6.90 (m, 3H), 3.90-3.80 (m, 5H), 3.76-3.74 (m, 2H), 3.55-3.50 (m, 2H), 3.32-3.29 (m, 2H), 2.77 (d, 3H, J=4.4 Hz), 1.80 (brs, 2H), 1.30 (s, 9H). LC-MS (Method B): (2M+H)⁺: 913.45; (M−H)⁺: 455.20; tRet: 1.38 min.

### Example 43B

### 2-(1,4-diazepan-1-yl)-4-((2-methoxyphenyl)amino)-N-methylpyrimidine-5-carboxamide (Table 1, Compound No. 29)

Synthesis is performed similar to N-t-Boc Deprotection and Examples above using acetyl chloride (14.0 eq), methanol, tert-butyl 4-(4-((2-methoxyphenyl)amino)-5-(methylcarbamoyl)pyrimidin-2-yl)-1,4-diazepane-1-carboxylate (1.0 eq) and stirring for 24 h at RT. After work-up, obtained solid is dissolved in water (2 mL) and dried under lyophilization to afford the desired compound as a white solid. Yield: 0.060 g. ¹H NMR (400 MHz) VT AT 90° C. DMSO-d6: δ=11.54 (brs, 1H), 9.23 (brs, 2H), 8.59 (s, 1H), 8.38-8.27 (m, 2H), 7.06-6.96 (m, 3H), 4.05 (brs, 2H), 3.87 (brs, 5H), 3.27-3.17 (m, 4H), 2.79 (s, 3H), 2.16 (brs, 2H). LC-MS (Method A): Parent MW: 356.20[M+H]⁻ tRet: 1.86 min: 357.30; HPLC: tRet: 5.13 min.

### Example 43C

### 2-(4-acetyl-1,4-diazepan-1-yl)-4-((2-methoxyphenyl)amino)-N-methylpyrimidine-5-carboxamide (Table 1, Compound No. 24)

Synthesis is performed similar to Amide Formation Reaction and Examples above using 2-(1,4-diazepan-1-yl)-4-((2-methoxyphenyl)amino)-N-methylpyrimidine-5-carboxamide hydrochloride (1.0 eq), DCM, pyridine (6.0 eq), and acetyl chloride (3.0 eq) initially at 0° C. and then stirring for 4 h at RT. After work-up, the solid is dissolved in acetonitrile (0.3 mL) and water (3 mL), freeze and dried under lyophilization to afford the desired compound as a white solid. Yield: 0.60 g, 74.22%. ¹H NMR (400 MHz) VT at 90° C. DMSO-d₆: 6=11.30 (s, 1H), 8.51 (s, 1H), 8.37 (brs, 1H), 8.03 (brs, 1H), 7.04-6.92 (m, 3H), 3.91-3.77 (m, 7H), 3.63-3.60 (m, 2H), 3.43 (brs, 2H), 2.78 (d, 3H, J=4.4 Hz), 2.02-1.76 (m, 5H). LC-MS (Method B): (2M+H)⁺: 797.35; (M−H)⁺: 397.15 tRet: 1.21 min. HPLC: 210 nm tRet: 5.69 min.

### Example 44

### Tert-Butyl 4-(S-carbamoyl-4-((2-(methylsulfonyl)phenyl)amino)pyrimidin-2-yl)-1,4-diazepane-1-carboxylate (Table 1, Compound No. 65)

Synthesis is performed similar to Second Heteroaryl Amination Reaction and Examples above and using Intermediate 8 (1.0 eq), BuOH, tert-butyl 1,4-diazepane-1-carboxylate (5.0 eq), and DIPEA (10.0 eq) and stirring for 1.5 h at 150° C. in microwave irradiation. Desired compound is a light yellow solid (0.4 g, 60% yield). LCMS (Method C): m/z=490.2 (M−H) tRet: 2.36 min, HPLC (Method F): tRet=7.017 min. ¹H NMR (400 MHz, DMSO-d₆): δ=11.42 (s, 1H), 8.65 (s, 1H), 8.21 (d, J=7.6 Hz, 1H), 7.90 (dd, J₁=8.0, J₂=1.6, 1H), 7.67-7.63 (m, 1H), 7.32-7.39 (m, 3H), 3.75-3.67 (m, 4H), 3.46 (t, J=5.2, 2H), 3.29 (t, J=5.6, 2H), 3.12 (s, 3H), 1.72 (bs, 2H), 1.5 (bs, 9H).

### Example 45

### 2-(1,4-diazepan-1-yl)-4-((2-(methylsulfonyl)phenyl)amino)pyrimidine-5-carboxamide hydrochloride (Table 1, Compound No. 79)

To a stirred solution of tert-butyl 4-(5-carbamoyl-4-((2-(methylsulfonyl)phenyl)amino)pyrimidin-2-yl)-1,4-diazepane-1-carboxylate (0.30 g, 0.611 mmol, 1.0 eq.) in DCM (5 ml) is added 2M HCl in 1,4-dioxane (5 ml) at 0° C. and stirred at RT for 2 h. Completion of the reaction is monitored by TLC. After completion of the reaction, the reaction mixture is evaporated completely under reduced pressure, washed with diethyl ether (10 ml×2) and dried under reduced pressure to afford the desired compound as an off-white solid (0.25 g, 96% yield). LCMS (Method B): m/z=391.10 (M+H)⁺ (Kinetex C18, 4.6×30 mm, 2.6 um, 100 A Mobile Phase A: 0.1% Formic Acid in water B: ACN. Method-T/% B-0/5, 0.5/95, 2.4/95, 2.5/5, 3.5/5 Flow rate 1.5 ml/Min). The fractions are lyophilized to afford the desired compound as a white solid (25 mg (formate salt), 33% yield). LCMS (Method B): (M+H)⁺ m/z=391.05, tRet: 1.10 min. HPLC (Method D): tRet: 5.30 min. ¹H NMR (400 MHz, DMSO-d₆) δ=11.43 (bs, 1H), 8.67 (s, 1H), 8.185 (d, J₁=8.4 Hz, 1H), 7.90 (dd, J₁=8.0, J₂=1.6 Hz, 1H), 7.67-7.63 (m, 1H), 7.34-7.29 (m, 3H), 3.84-3.76 (m, 4H), 3.13 (s, 3H), 3.08-3.05 (m, 2H), 2.99 (t, J₁=5.6 Hz, 2H), 1.93-1.87 (m, 2H).

### Example 46

### 2-(4-acetyl-1,4-diazepan-1-yl)-4-((2-(methylsulfonyl)phenyl)amino)pyrimidine-5-carboxamide (Table 1, Compound No. 80)

Synthesis is performed similar to Amide Formation Reaction and Examples above using 2-(1,4-diazepan-1-yl)-4-((2-(methylsulfonyl)phenyl)amino)pyrimidine-5-carboxamide hydrochloride (1.0 eq), DCM, TEA (6 eq), and acetyl chloride (1.3 eq) initially at 0° C. and then stirring at RT for 2 h. Desired compound is a white solid (75 mg, 50% yield). LCMS (Method C): m/z=433.2 (M+H)⁺ tRet: 1.94 min, HPLC (Method A): tRet: 5.16 min. ¹H NMR (400 MHz, DMSO-d₆): δ=11.39 (s, 1H), 8.64 (s, 1H) 8.15 (bs, 1H), 7.90 (d, J₁=7.6 Hz, 1H), 7.66 (t, J₁=7.6 Hz, 1H), 7.34-7.30 (m, 3H), 3.76-3.70 (m, 4H), 3.55 (bs, 2H), 3.41 (bs, 2H), 3.11 (s, 3H), 1.95-1.86 (m, 3H), 1.78-1.70 (m, 2H).

### Example 47

### Tert-Butyl 4-(S-(methylcarbamoyl)-4-((2-(methylsulfonyl)phenyl)amino)pyrimidin-2-yl)-1,4-diazepane-1-carboxylate (Table 1, Compound No. 66)

Synthesis is performed similar to Second Heteroaryl Amination Reaction and Examples above and using Intermediate 9 (1.0 eq), BuOH, tert-butyl 1,4-diazepane-1-carboxylate (3.0 eq), and DIPEA (5.0 eq) and stirring for 1.5 h at 150° C. in microwave irradiation. Desired compound is an off white solid (250 mg, 80% yield). LCMS (Method A): m/z=505.3 (M+H)⁺, tRet: 2.377 min; HPLC (Method D): tRet: 6.866 min. ¹H NMR (400 MHz, DMSO-d₆) δ=11.23 (s, 1H), 8.58 (m, 1H), 8.17 (d, J=8 Hz, 1H), 8.08 (bs, 1H), 7.90 (dd, J₁=8.0 Hz, J₂=1.2 Hz, 1H), 7.67-7.63 (m, 1H), 7.31 (t, J=7.2 Hz, 1H), 3.75-3.66 (m, 4H), 3.45 (t, J=5.6 Hz, 2H), 3.28 (t, J=6.0 Hz, 2H), 3.20 (s, 3H), 2.77 (d, J=4.8 Hz, 3H), 1.72 (bs, 2H), 1.32 (s, 9H).

### Example 48

### Example 48A

### 2-(1,4-diazepan-1-yl)-N-methyl-4-((2-(methylsulfonyl)phenyl)amino)pyrimidine-5-carboxamide hydrochloride (Table 1, Compound No. 67)

Synthesis is performed similar to N-t-Boc Deprotection and Examples above using tert-butyl 4-(5-(methylcarbamoyl)-4-((2-(methylsulfonyl)phenyl)amino)pyrimidin-2-yl)-1,4-diazepane-1-carboxylate (1.0 eq), DCM, and HCl in 1,4-dioxane (10.0 eq) and stirring for 2 h at RT. Desired compound is an off white solid (150 mg, 86% yield). LCMS (Method B): m/z=405.10 (M+H).

### Example 48B

### 2-(4-acetyl-1,4-diazepan-1-yl)-N-methyl-4-((2-(methylsulfonyl)phenyl)amino)pyrimidine-5-carboxamide (Table 1, Compound No. 75)

Synthesis is performed similar to Amide Formation Reaction and Examples above using 2-(1,4-diazepan-1-yl)-N-methyl-4-((2-(methylsulfonyl)phenyl)amino)pyrimidine-5-carboxamide hydrogen chloride (1.0 eq), DCM, triethylamine (5.0 eq), and acetyl chloride (2.0 eq) initially at 0° C. and then stirring for 2 h at RT. Desired compound is an off white solid (50 mg, 49% yield). LCMS (Method A): m/z=447.2 (M+H)⁺ tRet: 1.980 min; HPLC (Method B): tRet: 5.553 min. ¹H NMR (400 MHz, DMSO-d₆): δ=11.20 (s, 1H), 8.57 (s, 1H), 8.11-8.09 (m, 2H), 7.90 (d, J=8.0 Hz, 1H), 7.65 (t, J=7.6 Hz, 1H), 7.32 (t, J=8.0 Hz, 1H), 3.83-3.68 (m, 4H), 3.75-3.66 (m, 4H), 3.54 (m, 2H), 3.40 (m, 2H), 3.13 (s, 3H), 2.78 (d, J=4.4 Hz, 3H), 1.95-1.86 (m, 3H), 1.77-1.69 (m, 2H).

### Example 49

### Ethyl 2,4-dichloropyrimidine-5-carboxylate

2,4-dichloropyrimidine-5-carbonyl chloride (4.0 g, 19.054 mmol, 1.0 eq) in ethanol (40 mL) is stirred for 1 h at RT. Completion of the reaction is monitored by TLC. The reaction mixture is partitioned between EtOAc (50 mL) and water (60 mL) and the aqueous layer is extracted with EtOAc (30 mL×2). The combined organic phases are washed with brine, dried over Na₂SO₄ and the solvent is removed under reduced pressure to afford the crude compound. The crude compound is purified by flash chromatography, eluting with 0-20% EtOAc in hexane to afford desired compound as a colorless liquid. LC-MS (Method B): (M+H)⁺: 241.05; tRet 2.50 min. ethyl 2-chloro-4-((6-cyclopropylpyridin-3-yl)amino)pyrimidine-5-carboxylate

To a stirred solution of ethyl 2,4-dichloropyrimidine-5-carboxylate (0.50 g, 2.272 mmol, 1.0 eq) in ethanol (10 mL) is added 6-cyclopropylpyridin-3-amine (0.304 g, 2.272 mmol, 1.0 eq) at RT and the clear solution is stirred for 16 h at RT. TLC shows unreacted reagent, so reaction is heated to 80° C. for 2 h. Progress of the reaction is monitored by TLC. The reaction mixture is partitioned between EtOAc (50 mL) and water (60 mL) and the aqueous layer is extracted with EtOAc (30 mL×2). The combined organic phases are washed with brine, dried over anhydrous Na₂SO₄ and the solvent is removed under reduced pressure to afford the crude compound. The crude compound is purified by using flash chromatography, eluting with 30-100% EtOAc in hexane to afford the desired compound as a low melting white solid. LC-MS (Method: B): (M+H)⁺: 319.00; tRet: 1.46 min.

### Ethyl 4-((6-cyclopropylpyridin-3-yl)amino)-2-(pyrrolidin-1-yl)pyrimidine-5-carboxylate

To a stirred solution of ethyl 2-chloro-4-((6-cyclopropylpyridin-3-yl)amino)pyrimidine-5-carboxylate (0.500 g, 1.571 mmol, 1.0 eq) in n-butanol (15 mL) is added pyrrolidine (0.393 mL, 4.715 mmol, 3.0 eq) at RT. The reaction mixture is heated to 100° C. for 2 h. Completion of the reaction is monitored by TLC. Excess solvent is removed under reduced pressure. The reaction mixture is partitioned between EtOAc (100 mL) and water (150 mL) and the aqueous layer is extracted with EtOAc (50 mL×2). The combined organic phases are washed with brine, dried over anhydrous Na₂SO₄ and the solvent is removed under reduced pressure to afford the crude compound. The crude compound is purified by using flash chromatography, eluting with 30-40% EtOAc in hexane to afford the desired compound as a low melting white solid. LC-MS (Method B): (M+H)⁺ 354.10; tRet: 1.37 min, HPLC (Method B): tRet: 6.39 min.

### Lithium 4-((6-cyclopropylpyridin-3-yl)amino)-2-(pyrrolidin-1-yl)pyrimidine-5-carboxylate

To a stirred solution of ethyl 4-((6-cyclopropylpyridin-3-yl)amino)-2-(pyrrolidin-1-yl)pyrimidine-5-carboxylate (0.080 g, 0.226 mmol, 1.0 eq) in EtOH:THF:H₂O (5 mL) is added LiOH·H₂O (0.047 g, 1.132 mmol, 5.0 eq) at RT. The clear solution is stirred for 16 h at RT. Completion of the reaction is monitored by TLC. The excess solvent is removed under reduced pressure to afford the desired compound as an off white solid. LC-MS (Method C): (M-Li+H)⁺: 326.2; tRet: 1.83 & 1.89 min.

### 4-((6-cyclopropylpyridin-3-yl)amino)-N-hydroxy-2-(pyrrolidin-1-yl)pyrimidine-5-carboxamide (Table 1, Compound No. 54)

To a stirred solution of 4-((6-cyclopropylpyridin-3-yl)amino)-2-(pyrrolidin-1-yl)pyrimidine-5-carboxylic acid (0.035 g, 0.107 mol, 1.0 eq) in DMF (2 mL) are added DIPEA (0.093 mL, 0.538 mmol, 5.0 eq) and HATU (0.062 g, 0.161 mmol, 1.5 eq). The mixture is stirred for 15 min then hydroxylaminehydrochloride (0.023 g, 0.322 mmol, 3.0 eq) is added at RT. The clear solution is stirred for 2 days at RT. Progress of the reaction is monitored by TLC. The reaction mixture is diluted with water (50 mL) and obtained solid is filtered and dried under vacuum to afford the desired compound as an off white solid. Yield: 0.016 g. ¹H NMR (400 MHz) DMSO-d₆: δ=11.1 (brs, 1H), 11.0 (s, 1H), 9.0 (brs, 1H), 8.68 (d, 1H), 8.42 (s, 1H) 8.0 (dd, 1H) 7.25 (d, 1H), 3.5 (m, 4H), 2.0 (m, 1H), 1.92 (m, 4H), 0.88-0.93 (m, 4H), LC-MS: (M+H)⁺: 341.2; tRet: 1.81 min, LC-MS (Method C): (M+H)⁺: 341.2; tRet: 1.81 min, HPLC: HPLC (Method C): tRet: 5.38 min.

### Example 50

### TYK2 JH2 Domain Binding Assay

Binding constants for compounds disclosed herein may be determined by the KINOMEscan® assay developed by DiscoveRx. KINOMEscan® is based on a competition binding assay that quantitatively measures the ability of a compound to compete with an immobilized, active-site directed ligand. The assay is performed by combining three components: DNA-tagged kinase; immobilized ligand; and a test compound. The ability of the test compound to compete with the immobilized ligand is measure via quantitative PCR of the DNA tag.

Binding constants for compounds disclosed herein against the JH2 domain are determined by the following protocol for a KINOMEscan® assay (DiscoveRx). A fusion protein of a partial length construct of human TYK2 (JH2domain-pseudokinase) (amino acids G556 to D888 based on reference sequence NP 003322.3) and the DNA binding domain of NFkB is expressed in transiently transfected HEK293 cells. From these HEK 293 cells, extracts are prepared in M-PER extraction buffer (Pierce) in the presence of Protease Inhibitor Cocktail Complete (Roche) and Phosphatase Inhibitor Cocktail Set II (Merck) per manufacturers' instructions. The TYK2 (JH2domain-pseudokinase) fusion protein is labeled with a chimeric double-stranded DNA tag containing the NFkB binding site (5′-GGGAATTCCC-3′) fused to an amplicon for qPCR readout, which is added directly to the expression extract (the final concentration of DNA-tag in the binding reaction is 0.1 nM).

Streptavidin-coated magnetic beads (Dynal M280) are treated with a biotinylated small molecule ligand for 30 minutes at room temperature to generate affinity resins for the binding assays. The liganded beads are blocked with excess biotin and washed with blocking buffer (SeaBlock (Pierce), 1% BSA, 0.05% Tween 20, 1 mM DTT) to remove unbound ligand and to reduce nonspecific binding.

The binding reaction is assembled by combining 15.75 μl of DNA-tagged kinase extract, 3.75 μl liganded affinity beads, and 0.18 μl test compound (PBS/0.05% Tween 20/10 mM DTT/0.1% BSA/2 pg/ml sonicated salmon sperm DNA). Extracts are used directly in binding assays without any enzyme purification steps at a >10,000-fold overall stock dilution (final DNA tagged enzyme concentration<0.1 nM). Extracts are loaded with DNA-tag and diluted into the binding reaction in a two-step process. First extracts are diluted 1:100 in 1× binding buffer (PBS/0.05% Tween 20/10 mM DTT/0.1% BSA/2 pg/ml sonicated salmon sperm DNA) containing 10 nM DNA-tag. This dilution is allowed to equilibrate at room temperature for 15 minutes and then subsequently diluted 1:100 in 1× binding buffer. Test compounds are prepared as 111× stocks in 100% DMSO. Kds are determined using an 11-point 3-fold compound dilution series with three DMSO control points. All compounds for Kd measurements are distributed by acoustic transfer (non-contact dispensing) in 100% DMSO. The compounds are then diluted directly into the assays such that the final concentration of DMSO is 0.9%. All reactions performed in polypropylene 384-well plates. Each is a final volume of 0.02 mL. Assays are incubated with shaking for 1 hour at room temperature. Then the beads are pelleted and washed with wash buffer (lx PBS, 0.05% Tween 20) to remove displaced kinase and test compound. The washed beads are re-suspended in elution buffer (lx PBS, 0.05% Tween 20, 0.5 μM non-biotinylated affinity ligand) and incubated at room temperature with shaking for 30 minutes. The kinase concentration in the eluates is measured by qPCR. qPCR reactions are assembled by adding 2.5 μL of kinase eluate to 7.5 μL of qPCR master mix containing 0.15 μM amplicon primers and 0.15 μM amplicon probe. The qPCR protocol consists of a 10-minute hot start at 95° C., followed by 35 cycles of 95° C. for 15 seconds, 60° C. for 1 minute.

Test compounds are prepared as 111× stocks in 100% DMSO. Kds are determined using an 11-point 3-fold compound dilution series with three DMSO control points. All compounds for Kd measurements are distributed by acoustic transfer (non-contact dispensing) in 100% DMSO. The compounds are then diluted directly into the assays such that the final concentration of DMSO is 0.9%. The Kds are determined using a compound top concentration of 30,000 nM. Kd measurements are performed in duplicate. Binding constants (Kds) are calculated with a standard dose-response curve using the Hill equation: Background+((Signal−Background)/(1+(KdHillSlope/DoseHillSlope))). The Hill Slope is set to −1. Curves are fitted using a non-linear least square fit with the Levenberg-Marquardt algorithm (Levenberg, K., A method for the solution of certain non-linear problems in least squares, Q. Appl Math. 2, 164-168 (1944)).

Results are presented in Table 3 and Table 4. For compound numbers, please see Table 1 in Formula 1.32 above.

TABLE 3

| Cmpd. | TYK JH2 | Cmpd. | TYK JH2 | Cmpd. | TYK JH2 |
|---|---|---|---|---|---|
| No. | (Kd, nM) | No. | (Kd, nM) | No. | Kd, nM) |
| 1 | 170 | 30 | 44000 | 57 | 49 |
| 2 | 12 | 31 | 4800 | 58 | 1800 |
| 3 | 54 | 32 | 17000 | 59 | 270 |
| 4 | 38 | 33 | 710 | 60 | 2 |
| 5 | 0.69^{a} | 34 | 760 | 61 | 15 |
| 6 | 430 | 35 | 710 | 62 | 120 |
| 7 | 3.9 | 36 | 660 | 63 | 1400 |
| 8 | 360 | 37 | 34 | 64 | 1400 |
| 9 | 240 | 38 | 7.8 | 65 | 2900 |
| 10 | 370 | 39 | 0.82 | 66 | 6300 |
| 11 | 180 | 40 | 3300 | 67 | 3400 |
| 12 | 800 | 41 | 19000 | 68 | 160 |
| 13 | 1000 | 42 | 3300 | 69 | 1800 |
| 14 | 19000 | 43 | 7500 | 70 | 4000 |
| 15 | 42000 | 44 | 2400 | 71 | 470 |
| 16 | 2.6 | 45 | 200 | 72 | 4500 |
| 17 | 21 | 46 | 6600 | 73 | 150 |
| 18 | 440 | 47 | 6700 | 74 | 800 |
| 19 | 8200 | 48 | 0.5 | 75 | 1300 |
| 20 | 2500 | 49 | 2.3 | 76 | 210 |
| 21 | 14000 | 50 | 110 | 77 | 170 |
| 22 | 11000 | 51 | 1500 | 78 | 330 |
| 23 | 1600 | 52 | 1.6 | 79 | 1700 |
| 24 | 4900 | 53 | 0.11 | 80 | 950 |
| 25 | 180 | 54 | 160 | 81 | 370 |
| 26 | 190 | 55 | 180 | | |
| 27 | 36 | 56 | 11000 | | |
| 28 | 2.3 | | | | |
| 29 | 4600 | | | | |

^{a}Average of two measurements (0.45 and 0.93).

A pyrimidinyl compound of Formula I may be particularly advantageous as a TYK2 inhibitor, exhibiting a lower Kd than a compound with a different heteroaryl core.

TABLE 4

| | TYK JH2 |
|---|---|
| Cmpd. | (Kd, nM) |
| 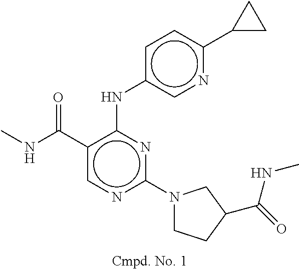 | 170 |
| 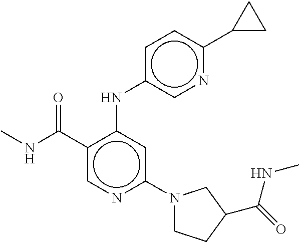 | 10000 |
| 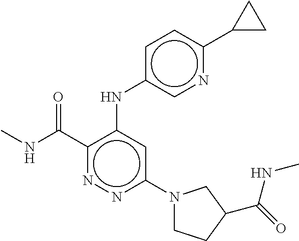 | 31000 |

### General Administration and Pharmaceutical Compositions

When used as pharmaceuticals, the compounds of the invention (e.g., any of Formula I or 1.1-1.37) are typically administered in the form of a pharmaceutical composition. Such compositions can be prepared using procedures well known in the pharmaceutical art and generally comprise at least one compound of the invention and at least one pharmaceutically acceptable carrier. The compounds of the invention may also be administered alone or in combination with adjuvants that enhance stability of the compounds of the invention, facilitate administration of pharmaceutical compositions containing them in certain embodiments, provide increased dissolution or dispersion, increased antagonist activity, provide adjunct therapy, and the like. The compounds according to the invention may be used on their own or in conjunction with other active substances according to the invention, optionally also in conjunction with other pharmacologically active substances. In general, the compounds of this invention are administered in a therapeutically or pharmaceutically effective amount but may be administered in lower amounts for diagnostic or other purposes.

Administration of the compounds of the invention (e.g., any of Formula I or 1.1-1.37), in pure form or in an appropriate pharmaceutical composition, can be carried out using any of the accepted modes of administration of pharmaceutical compositions. Thus, administration can be, for example, orally, buccally (e.g., sublingually), nasally, parenterally, topically, transdermally, vaginally, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, preferably in unit dosage forms suitable for simple administration of precise dosages. The pharmaceutical compositions will generally include a conventional pharmaceutical carrier or excipient and a compound of the invention as the/an active agent, and, in addition, may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration are well-known to those of skill in the art. The state of the art is evidenced, e.g., by Remington: The Science and Practice of Pharmacy, 20th Edition, A. Gennaro (ed.), Lippincott Williams & Wilkins, 2000; Handbook of Pharmaceutical Additives, Michael & Irene Ash (eds.), Gower, 1995; Handbook of Pharmaceutical Excipients, A. H. Kibbe (ed.), American Pharmaceutical Ass'n, 2000; H. C. Ansel and N. G. Popovish, Pharmaceutical Dosage Forms and Drug Delivery Systems, 5th ed., Lea and Febiger, 1990; each of which is incorporated herein by reference in their entireties to better describe the state of the art. As one of skill in the art would expect, the forms of the compounds of the invention utilized in a particular pharmaceutical formulation will be selected (e.g., salts) that possess suitable physical characteristics (e.g., water solubility) that are required for the formulation to be efficacious.

1. A compound of Formula I: 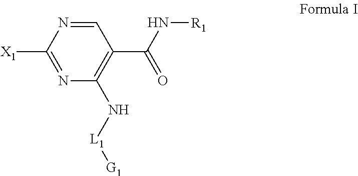
wherein:
R₁ is H, OH, or C₁₋₃-alkyl;
L₁ is a bond or carbonyl;
G₁ is an aryl or a 6- to 9-membered heteroaryl each optionally substituted with one or more substituents selected from —SC₁₋₃-alkyl, —SO₂C₁₋₃-alkyl, C₁₋₃-alkoxy, C₃₋₆-cycloalkyl, —C(O)R₂, halogen, and 5- to 6-membered heteroaryl optionally substituted with one or more halogens;
R₂ is a 5- to 6-membered saturated heterocyclyl;
X₁ is 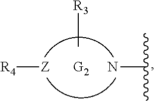
wherein the wavy line is the point of attachment of the fragment to Formula I;
G₂ is a 4- to 12-membered saturated heterocyclyl;
Z is CH or N;
R₃ is H or C₁₋₃-alkyl;
R₄ is H or —C(O)R₅; and
R₅ is C₁₋₃-alkyl, OH, NH₂, —NHC₁₋₃-alkyl, C₁₋₆-alkoxy, or C₃₋₆-cycloalkyl;
in free or pharmaceutically acceptable salt form.

2. The compound according to claim 1, in free or pharmaceutically acceptable salt form, wherein R₁ is —CH₃.

3. The compound according to claim 1, in free or pharmaceutically acceptable salt form, wherein L₁ is a bond.

4. The compound according to claim 1, in free or pharmaceutically acceptable salt form, wherein G₁ is pyridinyl, phenyl, or indazolyl.

5. The compound according to claim 4, in free or pharmaceutically acceptable salt form, wherein G₁ is pyridinyl.

6. The compound according to claim 5, in free or pharmaceutically acceptable salt form, wherein the pyridinyl is mono-substituted with cyclopropyl.

7. The compound according to claim 5, in free or pharmaceutically acceptable salt form, wherein the pyridinyl is: 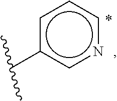
wherein the wavy line shows the point of attachment of the fragment to Formula I and the asterisk shows the carbon with substitution.

8. The compound according to claim 1, in free or pharmaceutically acceptable salt form, wherein G₁ is: 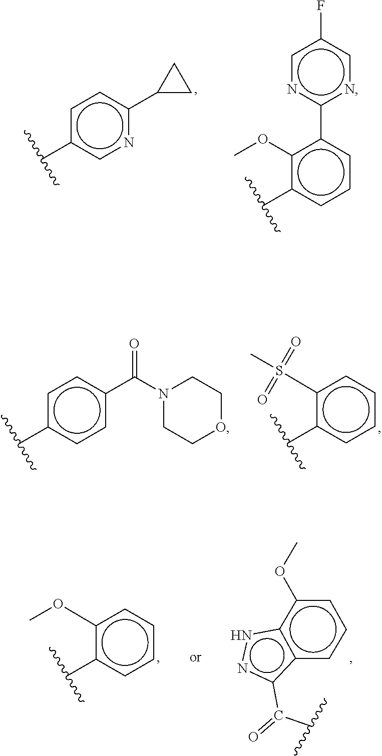
wherein the wavy line shows the point of attachment of the fragment to Formula I.

9. The compound according to claim 1, in free or pharmaceutically acceptable salt form, wherein G₂ is pyrrolidinyl, piperazinyl, piperdinyl, diazepanyl, or azetidinyl.

10. The compound according to claim 1, in free or pharmaceutically acceptable salt form, wherein R₄ is —C(O)R₅.

11. The compound according to claim 10, in free or pharmaceutically acceptable salt form, wherein R₅ is —NHCH₃.

12. The compound according to claim 1, in free or pharmaceutically acceptable salt form, wherein

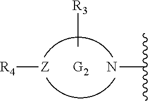

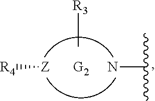

13. The compound according to claim 1, in free or pharmaceutically acceptable salt form, wherein 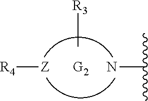
is: 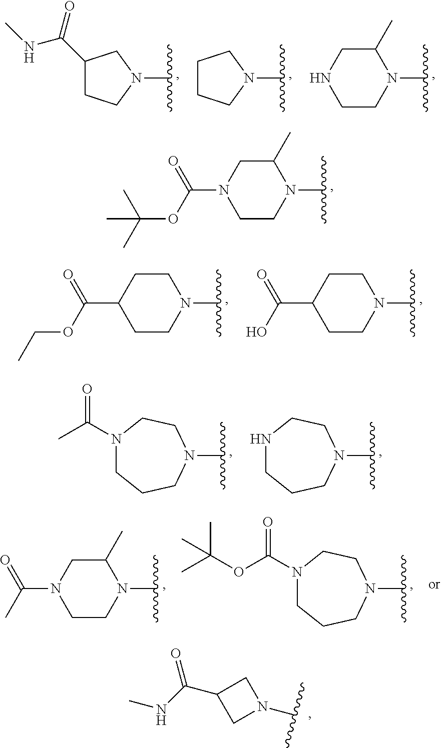
wherein the wavy line shows the point of attachment of the fragment to Formula I.

14. The compound according to claim 1, in free or pharmaceutically acceptable salt form, wherein 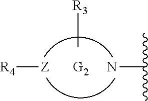
is:

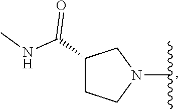 [SMILES: *N1CC[C@H](C(=O)NC)C1]

15. The compound according to claim 1, wherein the compound is selected from those set forth in Table 1 in Formula 1.32 or Table 2 in Formula 1.33, any in free or pharmaceutically acceptable salt form.

16. A pharmaceutical composition, wherein the pharmaceutical composition comprises the compound according to claim 1, in free or pharmaceutically acceptable salt form, in combination with pharmaceutically acceptable carrier.

17. A method for prophylaxis or treatment of a TYK2-mediated disorder in a patient in need thereof, wherein the method comprises administering an effective amount of the compound according to claim 1, in free or pharmaceutically acceptable salt form, to the patient.
